(19) United States
(12) Patent Application Publication
(10) Pub. No.: US 2026/0306468 A1
(43) Pub. Date: Oct. 8, 2026

(54) POLYMORPHIC FORMS AND FORMULATIONS OF LERIGLITAZONE

(71) Applicant: MINORYX THERAPEUTICS S.L., Mataró Barcelona (ES)

(72) Inventors: José Luis FÁBREGAS VIDAL, Barcelona (ES); Montserrat MARTÍ PÉREZ, Barcelona (ES)

(21) Appl. No.: 19/482,358

(22) PCT Filed: May 9, 2024
(86) PCT No.: PCT/IB2024/054534

§ 371 Date: Nov. 7, 2025

(30) Foreign Application Priority Data
May 9, 2023 (EP) 23382428.3

(51) Int. Cl. A61K 31/4439; A61K 9/00; A61K 9/08; A61K 47/12; A61K 47/22; A61K 47/26; A61K 47/38; C07D 417/12

(52) U.S. Cl. CPC A61K 31/4439; A61K 9/0053; A61K 9/08; A61K 47/12; A61K 47/22; A61K 47/26; A61K 47/38; C07D 417/12

(57) ABSTRACT

This disclosure provides crystalline forms of leriglitazone, pharmaceutical compositions or formulations comprising crystalline forms of leriglitazone, and methods of treating a disease, condition, or disorder in a patient comprising administering a composition comprising crystalline forms of leriglitazone to the patient.

### BACKGROUND

### Field of Invention

This disclosure provides crystalline forms of leriglitazone, pharmaceutical compositions or formulations comprising crystalline forms of leriglitazone, and methods of treating a disease, condition, or disorder in a patient comprising administering a composition comprising crystalline forms of leriglitazone to the patient.

### Background

5-[[4-[2-[5-(1-hydroxyethyl)pyridin-2-yl]ethoxy]phenyl]methyl]-1,3-thiazolidine-2,4-dione (referred to herein as “leriglitazone”) is a metabolite of pioglitazone, see, e.g., Sohda et al., Chem. Pharm. Bull. 43 (12): 2168-2172 (1995); Maeshiba et al., Arzneim.-Forsch/Drug Res. 47 (I): 29-35 (1997) having selective peroxisome proliferator-activated receptor gamma (PPAR-γ) agonist activity. WO2015/150476 A1 discloses leriglitazone for use in the treatment of central nervous system diseases. WO2018/100557 discloses leriglitazone, for the treatment of nonalcoholic fatty liver disease (“NAFLD”), nonalcoholic steatohepatitis (“NASH”), and other diseases and disorders. WO2018/116281 discloses methods for preparing leriglitazone. WO2019/234689 discloses an algorithm-based method to administer leriglitazone based on steady-state plasma levels of leriglitazone and related pharmacokinetic parameters in a patient.

### BRIEF SUMMARY

There exists a need for solid forms and formulations of leriglitazone for use in treatment of central nervous system diseases, nonalcoholic fatty liver disease (“NAFLD”), nonalcoholic steatohepatitis (“NASH”), and other diseases, disorders, and conditions in a patient, e.g., a human.

In one aspect, the present disclosure provides crystalline polymorphic forms of leriglitazone.

In another aspect, the present disclosure provides methods of making crystalline polymorphic forms of leriglitazone.

In another aspect, the present disclosure provides pharmaceutical compositions comprising crystalline polymorphic forms of leriglitazone and one or more pharmaceutically acceptable carriers, diluents or excipients.

In another aspect, the present disclosure provides pharmaceutical compositions or formulations comprising crystalline polymorphic forms of leriglitazone and water.

In another aspect, the present disclosure provides a method of using crystalline polymorphic forms or formulations of leriglitazone to treat a disease, disorder, or condition, in a subject.

In another aspect, the present disclosure provides crystalline polymorphic forms of leriglitazone or formulations thereof for use to treat a disease, disorder, or condition, which comprises administering to a subject in need thereof, including a human, crystalline polymorphic forms or formulations of leriglitazone as defined herein and, optionally, one or more pharmaceutically acceptable carriers, diluents or excipients.

In another aspect, the present disclosure provides compositions comprising leriglitazone, leriglitazone hydrochloride, or leriglitazone hydrate and (i) (Z)-5-(4-(2-(5-(1-hydroxyethyl)pyridin-2-yl)ethoxy)benzylidene)thiazolidine-2,4-dione, or the hydrochloride salt thereof; (ii) 2,2′-disulfanediylbis(3-(4-(2-(5-(1-hydroxyethyl)pyridin-2-yl)ethoxy)phenyl) propanoic acid), or the hydrochloride salt thereof; (iii) 2-((1-hydroxy-3-(4-(2-(5-(1-hydroxyethyl)pyridin-2-yl)ethoxy)phenyl) propan-2-yl)disulfaneyl)-3-(4-(2-(5-(1-hydroxyethyl)pyridin-2-yl)ethoxy)phenyl) propanoic acid, or the hydrochloride salt thereof; and/or (iv) 5-(4-(2-(5-(1-hydroxyethyl)pyridin-2-yl)ethoxy)benzyl) thiazolidin-2-one, or the hydrochloride salt thereof.

### BRIEF DESCRIPTION OF DRAWINGS

FIG. 1 is a XRPD diffractogram of leriglitazone hydrochloride Form A.

FIG. 2 is a Raman spectrum of leriglitazone hydrochloride Form A.

FIG. 3 is a crystal structure including disorder of leriglitazone hydrochloride Form A

FIGS. 4a-4d are crystal structures of different stereoisomers of leriglitazone hydrochloride Form A

FIG. 5 is a DSC thermogram of leriglitazone hydrochloride Form A.

FIG. 6 is a XRPD diffractogram of leriglitazone Form B.

FIG. 7 is a DSC thermogram of leriglitazone Form B.

FIG. 8 is a XRPD diffractogram of leriglitazone hydrate Form C.

FIG. 9 is a Raman spectrum of leriglitazone hydrate Form C.

FIG. 10 is a DSC thermogram of leriglitazone hydrate Form C.

FIG. 11 is two XRPD diffractograms comparing leriglitazone Form B (upper diffractogram) and leriglitazone Form C (lower diffractogram).

FIG. 12 is an illustration showing steps 1-7 of the manufacturing process flow chart used to prepare a pharmaceutical formulation comprising leriglitazone hydrate Form C.

FIG. 13 is an illustration showing steps 8-14 of the manufacturing process flow chart used to prepare a pharmaceutical formulation comprising leriglitazone hydrate Form C. “MIN-102” refers to Form A.

FIG. 14 is an illustration showing steps 15-20 of the manufacturing process flow chart used to prepare a pharmaceutical formulation comprising leriglitazone hydrate Form C.

FIG. 15 is two HPLC chromatograms of a leriglitazone oral suspension at pH 4 (upper) and pH 5.5 (lower).

### DETAILED DESCRIPTION

In one embodiment, the present disclosure provides a crystalline polymorphic form of leriglitazone hydrochloride, a crystalline polymorphic form of leriglitazone, or a crystalline polymorphic form of leriglitazone hydrate, collectively referred to as “leriglitazone polymorphs” or individually as a “leriglitazone polymorph.”

In another embodiment, the leriglitazone polymorph is a crystalline hydrochloride salt of leriglitazone, i.e., leriglitazone hydrochloride, represented by the formula: leriglitazone·HCl. This polymorph is referred to as “leriglitazone hydrochloride Form A” or simply “Form A.”

In another embodiment, the leriglitazone polymorph is a crystalline unsolvated free base of leriglitazone. This polymorph is referred to as “leriglitazone Form B” or simply “Form B.”

In another embodiment, the leriglitazone polymorph is a crystalline leriglitazone hydrate represented by the formula: leriglitazone·xH₂O, wherein x is 0.5 to 3. This polymorph is referred to as “leriglitazone hydrate Form C” or simply “Form C.”

### I. CRYSTALLINE LERIGLITAZONE HYDROCHLORIDE FORM A

In one embodiment, Form A is characterized as having a x-ray powder diffraction (XRPD) pattern with peaks at 10.8, 12.4, 20.5, 21.7, and 26.1 degrees 2θ using Cu Kα radiation, wherein the 2Θ values are ±0.2 degrees 2Θ.

In another embodiment, Form A is characterized as having a XRPD pattern with at least three peaks at 8.8, 9.2, 10.8, 12.4, 12.7, 15.5, 16.5, 17.0, 17.4, 17.7, 18.5, 19.7, 20.0, 20.5, 20.8, 21.7, 22.4, 22.7, 23.0, 24.3, 24.9, 25.2, 25.5, 26.1, 26.7, 27.0, 27.8, 28.3, 28.7, 29.3, 29.7, 29.9, 30.1, 31.2, 31.7, 31.9, 32.3, 32.7, 33.2, 33.5, 33.8, 34.1, 34.3, 35.0, 35.2, 35.6, 35.9, 36.4, 37.1, 37.5, 37.9, 38.2, 38.7, 39.0, 39.7, and/or 40.3 degrees 2Θ using Cu Kα radiation, wherein the 2Θ values are ±0.2 degrees 2Θ.

In another embodiment, Form A is characterized as having a XRPD pattern with at least four peaks at 8.8, 9.2, 10.8, 12.4, 12.7, 15.5, 16.5, 17.0, 17.4, 17.7, 18.5, 19.7, 20.0, 20.5, 20.8, 21.7, 22.4, 22.7, 23.0, 24.3, 24.9, 25.2, 25.5, 26.1, 26.7, 27.0, 27.8, 28.3, 28.7, 29.3, 29.7, 29.9, 30.1, 31.2, 31.7, 31.9, 32.3, 32.7, 33.2, 33.5, 33.8, 34.1, 34.3, 35.0, 35.2, 35.6, 35.9, 36.4, 37.1, 37.5, 37.9, 38.2, 38.7, 39.0, 39.7, and/or 40.3 degrees 2Θ using Cu Kα radiation, wherein the 2Θ values are ±0.2 degrees 2Θ.

In another embodiment, Form A is characterized as having a XRPD pattern with at least five peaks at 8.8, 9.2, 10.8, 12.4, 12.7, 15.5, 16.5, 17.0, 17.4, 17.7, 18.5, 19.7, 20.0, 20.5, 20.8, 21.7, 22.4, 22.7, 23.0, 24.3, 24.9, 25.2, 25.5, 26.1, 26.7, 27.0, 27.8, 28.3, 28.7, 29.3, 29.7, 29.9, 30.1, 31.2, 31.7, 31.9, 32.3, 32.7, 33.2, 33.5, 33.8, 34.1, 34.3, 35.0, 35.2, 35.6, 35.9, 36.4, 37.1, 37.5, 37.9, 38.2, 38.7, 39.0, 39.7, and/or 40.3 degrees 2Θ using Cu Kα radiation, wherein the 2Θ values are ±0.2 degrees 2Θ.

In another embodiment, Form A is characterized as having a XRPD pattern with at least six peaks at 8.8, 9.2, 10.8, 12.4, 12.7, 15.5, 16.5, 17.0, 17.4, 17.7, 18.5, 19.7, 20.0, 20.5, 20.8, 21.7, 22.4, 22.7, 23.0, 24.3, 24.9, 25.2, 25.5, 26.1, 26.7, 27.0, 27.8, 28.3, 28.7, 29.3, 29.7, 29.9, 30.1, 31.2, 31.7, 31.9, 32.3, 32.7, 33.2, 33.5, 33.8, 34.1, 34.3, 35.0, 35.2, 35.6, 35.9, 36.4, 37.1, 37.5, 37.9, 38.2, 38.7, 39.0, 39.7, and/or 40.3 degrees 2Θ using Cu Kα radiation, wherein the 2Θ values are ±0.2 degrees 2Θ.

In another embodiment, Form A is characterized as having a XRPD pattern with at least seven peaks at 8.8, 9.2, 10.8, 12.4, 12.7, 15.5, 16.5, 17.0, 17.4, 17.7, 18.5, 19.7, 20.0, 20.5, 20.8, 21.7, 22.4, 22.7, 23.0, 24.3, 24.9, 25.2, 25.5, 26.1, 26.7, 27.0, 27.8, 28.3, 28.7, 29.3, 29.7, 29.9, 30.1, 31.2, 31.7, 31.9, 32.3, 32.7, 33.2, 33.5, 33.8, 34.1, 34.3, 35.0, 35.2, 35.6, 35.9, 36.4, 37.1, 37.5, 37.9, 38.2, 38.7, 39.0, 39.7, and/or 40.3 degrees 2Θ using Cu Kα radiation, wherein the 2Θ values are ±0.2 degrees 2Θ.

In another embodiment, Form A is characterized as having a XRPD pattern with at least eight peaks at 8.8, 9.2, 10.8, 12.4, 12.7, 15.5, 16.5, 17.0, 17.4, 17.7, 18.5, 19.7, 20.0, 20.5, 20.8, 21.7, 22.4, 22.7, 23.0, 24.3, 24.9, 25.2, 25.5, 26.1, 26.7, 27.0, 27.8, 28.3, 28.7, 29.3, 29.7, 29.9, 30.1, 31.2, 31.7, 31.9, 32.3, 32.7, 33.2, 33.5, 33.8, 34.1, 34.3, 35.0, 35.2, 35.6, 35.9, 36.4, 37.1, 37.5, 37.9, 38.2, 38.7, 39.0, 39.7, and/or 40.3 degrees 2Θ using Cu Kα radiation, wherein the 2Θ values are ±0.2 degrees 2Θ.

In another embodiment, Form A is characterized as having a XRPD pattern with at least nine peaks at 8.8, 9.2, 10.8, 12.4, 12.7, 15.5, 16.5, 17.0, 17.4, 17.7, 18.5, 19.7, 20.0, 20.5, 20.8, 21.7, 22.4, 22.7, 23.0, 24.3, 24.9, 25.2, 25.5, 26.1, 26.7, 27.0, 27.8, 28.3, 28.7, 29.3, 29.7, 29.9, 30.1, 31.2, 31.7, 31.9, 32.3, 32.7, 33.2, 33.5, 33.8, 34.1, 34.3, 35.0, 35.2, 35.6, 35.9, 36.4, 37.1, 37.5, 37.9, 38.2, 38.7, 39.0, 39.7, and/or 40.3 degrees 2Θ using Cu Kα radiation, wherein the 2Θ values are ±0.2 degrees 2Θ.

In another embodiment, Form A is characterized as having a XRPD pattern with at least ten peaks at 8.8, 9.2, 10.8, 12.4, 12.7, 15.5, 16.5, 17.0, 17.4, 17.7, 18.5, 19.7, 20.0, 20.5, 20.8, 21.7, 22.4, 22.7, 23.0, 24.3, 24.9, 25.2, 25.5, 26.1, 26.7, 27.0, 27.8, 28.3, 28.7, 29.3, 29.7, 29.9, 30.1, 31.2, 31.7, 31.9, 32.3, 32.7, 33.2, 33.5, 33.8, 34.1, 34.3, 35.0, 35.2, 35.6, 35.9, 36.4, 37.1, 37.5, 37.9, 38.2, 38.7, 39.0, 39.7, and/or 40.3 degrees 2Θ using Cu Kα radiation, wherein the 2Θ values are ±0.2 degrees 2Θ.

In another embodiment, Form A is characterized as having a XRPD pattern with peaks at 8.8, 9.2, 10.8, 12.4, 12.7, 15.5, 16.5, 17.0, 17.4, 17.7, 18.5, 19.7, 20.0, 20.5, 20.8, 21.7, 22.4, 22.7, 23.0, 24.3, 24.9, 25.2, 25.5, 26.1, 26.7, 27.0, 27.8, 28.3, 28.7, 29.3, 29.7, 29.9, 30.1, 31.2, 31.7, 31.9, 32.3, 32.7, 33.2, 33.5, 33.8, 34.1, 34.3, 35.0, 35.2, 35.6, 35.9, 36.4, 37.1, 37.5, 37.9, 38.2, 38.7, 39.0, 39.7, and/or 40.3 degrees 2Θ using Cu Kα radiation, wherein the 2Θ values are ±0.2 degrees 2Θ.

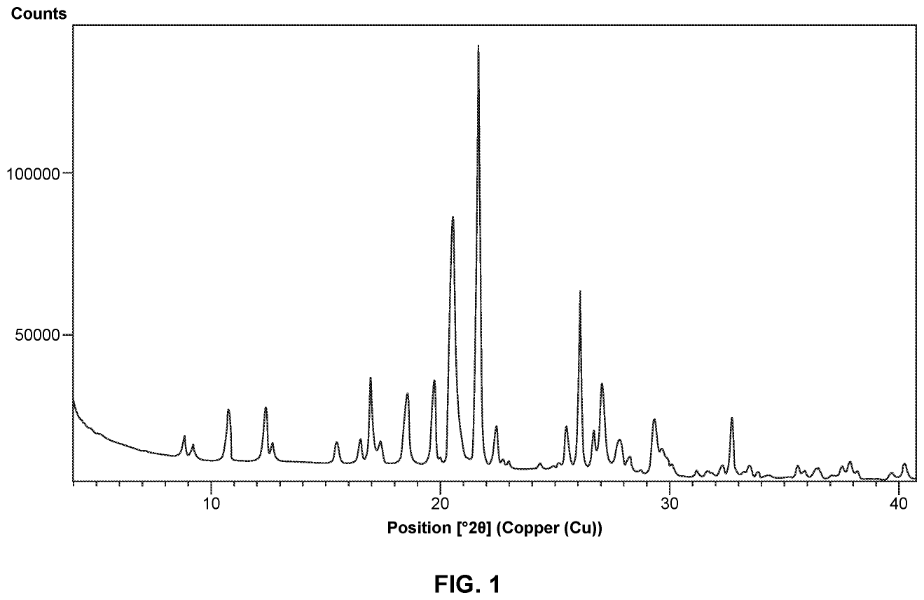

In another embodiment, Form A is characterized as having a XRPD diffractogram that is essentially the same as the one depicted in FIG. 1.

In another embodiment, Form A is characterized as having a FT Raman spectrum with peaks at 3307, 2925, 1748, 1642, 1612, 870, 638, 605, 116 cm⁻¹, wherein the cm⁻¹ values are ±4 cm⁻¹.

In another embodiment, Form A, characterized as having an FT-Raman spectrum with peaks at 3307, 3096, 3065, 2970, 2925, 2871, 1748, 1685, 1642, 1612, 1585, 1436, 1327, 1310, 1244, 1207, 1184, 1153, 1088, 1070, 1034, 936, 908, 870, 854, 789, 737, 707, 646, 638, 605, 497, 469, 437, 380, 333, 309, 221, and 116 cm⁻¹, wherein the cm⁻¹ values are ±4 cm⁻¹.

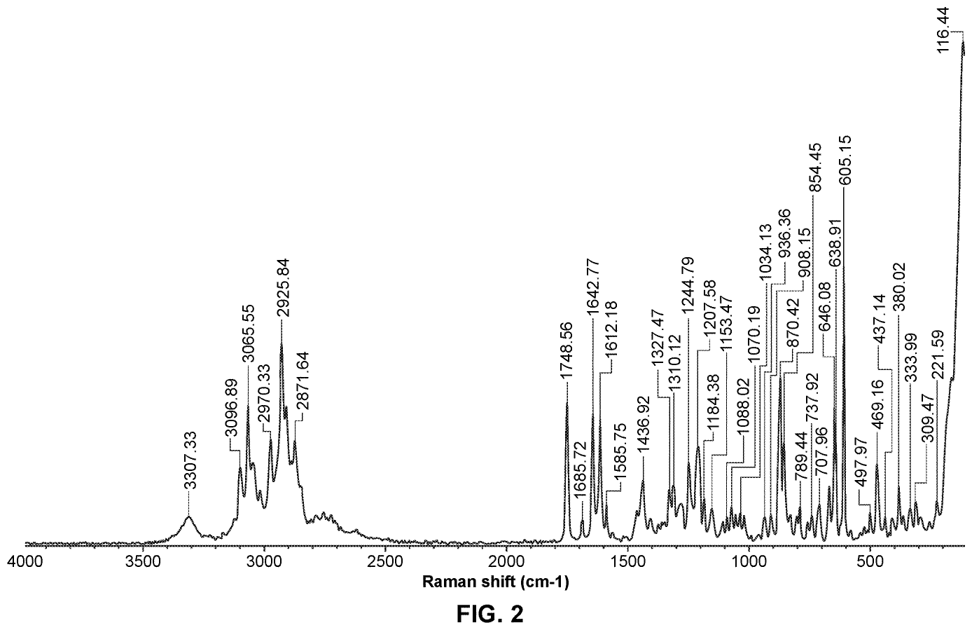

In another embodiment, Form A is characterized as having a FT-Raman spectrum that is essentially the same as the one depicted in FIG. 2.

In another embodiment, Form A is characterized as having a melting point with an onset temperature of about 203.98° C. and a peak temperature of about 211.87° C. based on differential scanning calorimetry.

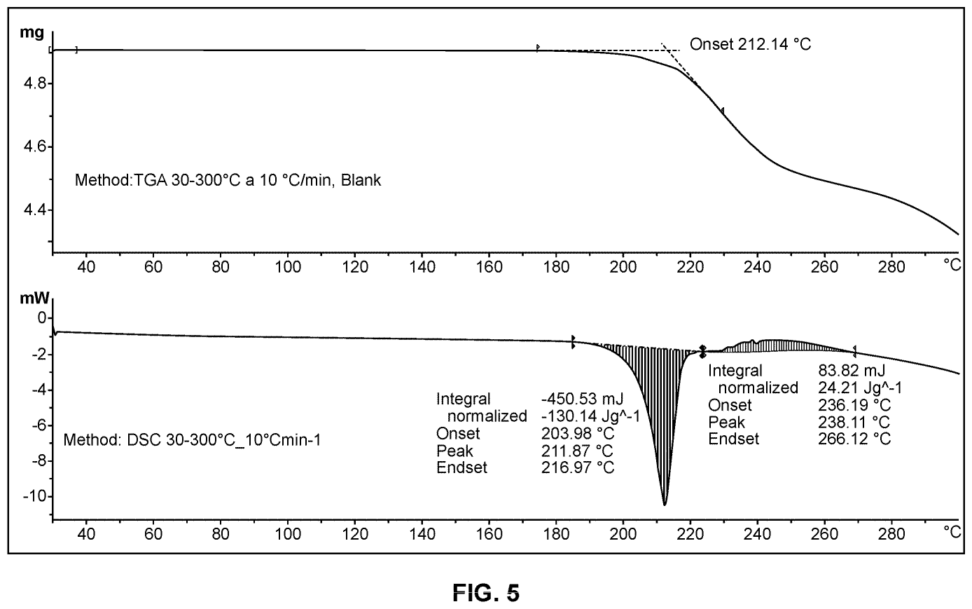

In another embodiment, Form A is characterized as having a DSC thermogram that is essentially the same as the one depicted in FIG. 5.

### II. CRYSTALLINE LERIGLITAZONE (UNSOLVATED FREE BASE) FORM B

In one embodiment, Form B is characterized as having a x-ray powder diffraction (XRPD) pattern with peaks at 9.0, 16.6 and 18.8 degrees 2Θ using Cu Kα radiation, wherein the 2Θ values are ±0.2 degrees 2Θ.

In another embodiment, Form B is characterized as having a powder x-ray diffraction (XRPD) pattern with peaks at 9.0, 16.6, 18.8, 21.1, 23.8, and 24.7 degrees 2Θ using Cu Kα radiation, wherein the 2Θ values are ±0.2 degrees 2Θ.

In another embodiment, Form B is characterized as having a XRPD pattern with peaks at 18.8, 21.1, and 23.8 degrees 2Θ using Cu Kα radiation, wherein the 2Θ values are ±0.2 degrees 2Θ.

In another embodiment, Form B is characterized as having a XRPD pattern with peaks at 16.6, 21.8, and 24.7 degrees 2Θ using Cu Kα radiation, wherein the 2Θ values are ±0.2 degrees 2Θ.

In another embodiment, Form B is characterized as having a XRPD pattern with peaks at 15.2, 25.4, and 28.0 degrees 2Θ using Cu Kα radiation, wherein the 2Θ values are ±0.2 degrees 2Θ.

In another embodiment, Form B is characterized as having a XRPD pattern with peaks at 9.0, 19.8, and 30.8 degrees 2Θ using Cu Kα radiation, wherein the 2Θ values are ±0.2 degrees 2Θ.

In another embodiment, Form B is characterized as having a XRPD pattern with peaks at 10.5, 20.1, and 26.9 degrees 2Θ using Cu Kα radiation, wherein the 2Θ values are ±0.2 degrees 2Θ.

In another embodiment, Form B is characterized as having a XRPD pattern with peaks at 12.4, 23.5, and 29.1 degrees 2Θ using Cu Kα radiation, wherein the 2Θ values are ±0.2 degrees 2Θ.

In another embodiment, Form B is characterized as having a XRPD pattern with at least three peaks at 7.6, 9.0, 10.5, 12.4, 15.2, 16.1, 16.6, 18.5, 18.8, 19.3, 19.8, 20.1, 20.5, 21.1, 21.8, 22.9, 23.2, 23.5, 23.8, 24.7, 25.4, 26.0, 26.5, 26.9, 27.4, 28.0, 28.9, 29.1, 29.5, 29.9, 30.8, 31.2, 31.5, 32.1, 32.6, 32.9, 33.7, 34.4, 35.4, 35.8, 36.3, 36.9, 37.1, 38.0, 38.4 and/or 39.3 degrees 2Θ using Cu Kα radiation, wherein the 2Θ values are ±0.2 degrees 2Θ.

In another embodiment, Form B is characterized as having a XRPD pattern with at least four peaks at 7.6, 9.0, 10.5, 12.4, 15.2, 16.1, 16.6, 18.5, 18.8, 19.3, 19.8, 20.1, 20.5, 21.1, 21.8, 22.9, 23.2, 23.5, 23.8, 24.7, 25.4, 26.0, 26.5, 26.9, 27.4, 28.0, 28.9, 29.1, 29.5, 29.9, 30.8, 31.2, 31.5, 32.1, 32.6, 32.9, 33.7, 34.4, 35.4, 35.8, 36.3, 36.9, 37.1, 38.0, 38.4 and/or 39.3 degrees 2Θ using Cu Kα radiation, wherein the 2Θ values are ±0.2 degrees 2Θ.

In another embodiment, Form B is characterized as having a XRPD pattern with at least five peaks at 7.6, 9.0, 10.5, 12.4, 15.2, 16.1, 16.6, 18.5, 18.8, 19.3, 19.8, 20.1, 20.5, 21.1, 21.8, 22.9, 23.2, 23.5, 23.8, 24.7, 25.4, 26.0, 26.5, 26.9, 27.4, 28.0, 28.9, 29.1, 29.5, 29.9, 30.8, 31.2, 31.5, 32.1, 32.6, 32.9, 33.7, 34.4, 35.4, 35.8, 36.3, 36.9, 37.1, 38.0, 38.4 and/or 39.3 degrees 2Θ using Cu Kα radiation, wherein the 2Θ values are ±0.2 degrees 2Θ.

In another embodiment, Form B is characterized as having a XRPD pattern with at least six peaks at 7.6, 9.0, 10.5, 12.4, 15.2, 16.1, 16.6, 18.5, 18.8, 19.3, 19.8, 20.1, 20.5, 21.1, 21.8, 22.9, 23.2, 23.5, 23.8, 24.7, 25.4, 26.0, 26.5, 26.9, 27.4, 28.0, 28.9, 29.1, 29.5, 29.9, 30.8, 31.2, 31.5, 32.1, 32.6, 32.9, 33.7, 34.4, 35.4, 35.8, 36.3, 36.9, 37.1, 38.0, 38.4 and/or 39.3 degrees 2Θ using Cu Kα radiation, wherein the 2Θ values are ±0.2 degrees 2Θ.

In another embodiment, Form B is characterized as having a XRPD pattern with at least seven peaks at 7.6, 9.0, 10.5, 12.4, 15.2, 16.1, 16.6, 18.5, 18.8, 19.3, 19.8, 20.1, 20.5, 21.1, 21.8, 22.9, 23.2, 23.5, 23.8, 24.7, 25.4, 26.0, 26.5, 26.9, 27.4, 28.0, 28.9, 29.1, 29.5, 29.9, 30.8, 31.2, 31.5, 32.1, 32.6, 32.9, 33.7, 34.4, 35.4, 35.8, 36.3, 36.9, 37.1, 38.0, 38.4 and/or 39.3 degrees 2Θ using Cu Kα radiation, wherein the 2Θ values are ±0.2 degrees 2Θ.

In another embodiment, Form B is characterized as having a XRPD pattern with at least eight peaks at 7.6, 9.0, 10.5, 12.4, 15.2, 16.1, 16.6, 18.5, 18.8, 19.3, 19.8, 20.1, 20.5, 21.1, 21.8, 22.9, 23.2, 23.5, 23.8, 24.7, 25.4, 26.0, 26.5, 26.9, 27.4, 28.0, 28.9, 29.1, 29.5, 29.9, 30.8, 31.2, 31.5, 32.1, 32.6, 32.9, 33.7, 34.4, 35.4, 35.8, 36.3, 36.9, 37.1, 38.0, 38.4 and/or 39.3 degrees 2Θ using Cu Kα radiation, wherein the 2Θ values are ±0.2 degrees 2Θ.

In another embodiment, Form B is characterized as having a XRPD pattern with at least nine peaks at 7.6, 9.0, 10.5, 12.4, 15.2, 16.1, 16.6, 18.5, 18.8, 19.3, 19.8, 20.1, 20.5, 21.1, 21.8, 22.9, 23.2, 23.5, 23.8, 24.7, 25.4, 26.0, 26.5, 26.9, 27.4, 28.0, 28.9, 29.1, 29.5, 29.9, 30.8, 31.2, 31.5, 32.1, 32.6, 32.9, 33.7, 34.4, 35.4, 35.8, 36.3, 36.9, 37.1, 38.0, 38.4 and/or 39.3 degrees 2Θ using Cu Kα radiation, wherein the 2Θ values are ±0.2 degrees 2Θ.

In another embodiment, Form B is characterized as having a XRPD pattern with at least ten peaks at 7.6, 9.0, 10.5, 12.4, 15.2, 16.1, 16.6, 18.5, 18.8, 19.3, 19.8, 20.1, 20.5, 21.1, 21.8, 22.9, 23.2, 23.5, 23.8, 24.7, 25.4, 26.0, 26.5, 26.9, 27.4, 28.0, 28.9, 29.1, 29.5, 29.9, 30.8, 31.2, 31.5, 32.1, 32.6, 32.9, 33.7, 34.4, 35.4, 35.8, 36.3, 36.9, 37.1, 38.0, 38.4 and/or 39.3 degrees 2Θ using Cu Kα radiation, wherein the 2Θ values are ±0.2 degrees 2Θ.

In another embodiment, Form B is characterized as having a XRPD pattern with peaks at 7.6, 9.0, 10.5, 12.4, 15.2, 16.1, 16.6, 18.5, 18.8, 19.3, 19.8, 20.1, 20.5, 21.1, 21.8, 22.9, 23.2, 23.5, 23.8, 24.7, 25.4, 26.0, 26.5, 26.9, 27.4, 28.0, 28.9, 29.1, 29.5, 29.9, 30.8, 31.2, 31.5, 32.1, 32.6, 32.9, 33.7, 34.4, 35.4, 35.8, 36.3, 36.9, 37.1, 38.0, 38.4 and/or 39.3 degrees 2Θ using Cu Kα radiation, wherein the 2Θ values are ±0.2 degrees 2Θ.

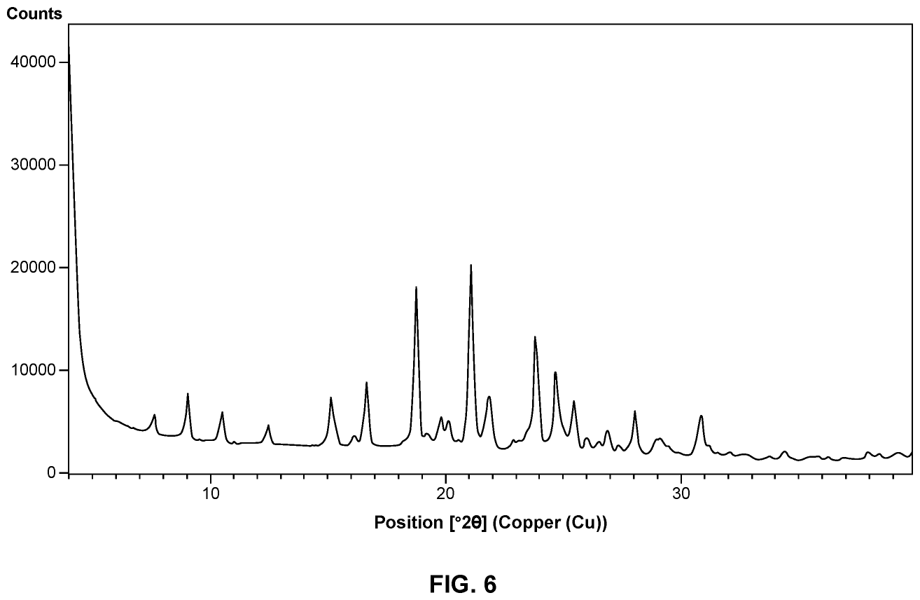

In another embodiment, Form B is characterized as having a XRPD diffractogram that is essentially the same as the one depicted in FIG. 6.

In another embodiment, Form B is characterized as having a melting point with an onset temperature of about 149.34° C. and a peak temperature of about 153.56° C. based on differential scanning calorimetry.

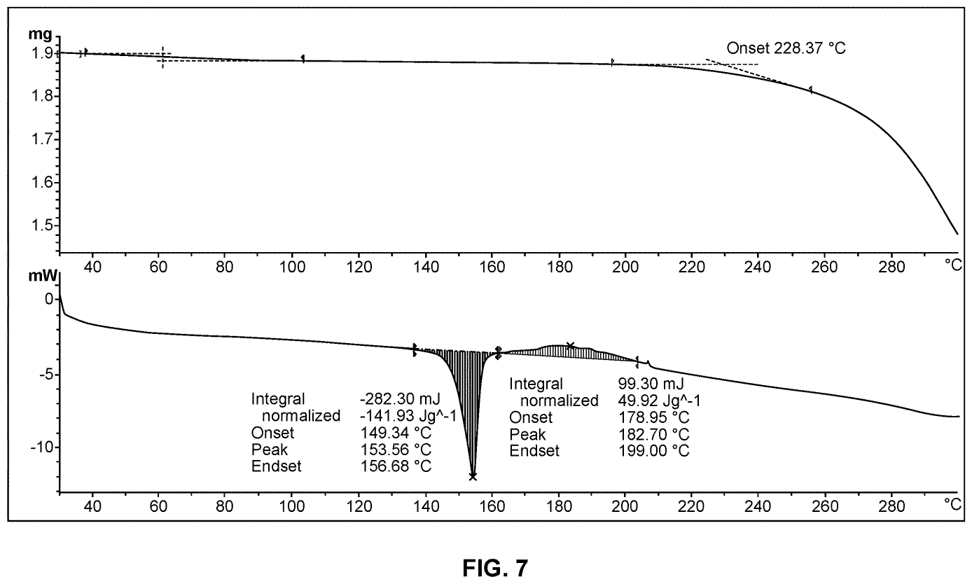

In another embodiment, Form B is characterized as having a DSC thermogram that is essentially the same as the one depicted in FIG. 7.

In another embodiment, Form B is characterized as having a TGA plot that is essentially the same as the one depicted in FIG. 7.

In another embodiment, Form B is characterized as having a TGA comprising a mass loss of less than about 1%, e.g., about 0.9%, e.g., about 0.05%, of the total mass of the sample upon heating from about 25° C. to about 200° C.

In another embodiment, Form B does not contain substantial amounts of either water or other solvent in the crystal lattice. In certain embodiments, Form B is unsolvated. In certain embodiments, Form B is anhydrous.

In another embodiment, Form B is characterized by its stability profile. In another embodiment, Form B material is stable, e.g., its XRPD pattern remains substantially unchanged, upon exposure to elevated temperature, upon exposure to elevated humidity, upon exposure to one or more solvents, and/or upon compression.

In another embodiment, the present disclosure provides a pharmaceutical composition comprising a suspension of a leriglitazone polymorph, e.g., leriglitazone (unsolvated free base) Form B, in water and, optionally, one or more pharmaceutically acceptable carriers, diluents or excipients.

### III. CRYSTALLINE LERIGLITAZONE HYDRATE FORM C

In one embodiment, Form C is characterized as having a XRPD pattern with peaks at 19.1, 21.0, and 23.8 degrees 2Θ using Cu Kα radiation, wherein the 2Θ values are ±0.2 degrees 2Θ.

In another embodiment, Form C is characterized as having a XRPD pattern with peaks at 12.7, 15.4, 19.1, 21.0, 23.8, and 25.9 degrees 2Θ using Cu Kα radiation, wherein the 2Θ values are ±0.2 degrees 2Θ.

In another embodiment, Form C is characterized as having a XRPD pattern with peaks at 12.7, 15.4, and 16.9 degrees 2Θ using Cu Kα radiation, wherein the 2Θ values are ±0.2 degrees 2Θ.

In another embodiment, Form C is characterized as having a XRPD pattern with peaks at 16.9, 21.3, and 21.6 degrees 2Θ using Cu Kα radiation, wherein the 2Θ values are ±0.2 degrees 2Θ.

In another embodiment, Form C is characterized as having a XRPD pattern with peaks at 7.7, 24.6, and 28.0 degrees 2Θ using Cu Kα radiation, wherein the 2Θ values are ±0.2 degrees 2Θ.

In another embodiment, Form C is characterized as having a XRPD pattern with peaks at 9.2, 19.8, 24.6, and 24.9 degrees 2Θ using Cu Kα radiation, wherein the 20 values are ±0.2 degrees 2Θ.

In another embodiment, Form C is characterized as having a XRPD pattern with peaks at 10.6, 24.9, and 27.3 degrees 2Θ using Cu Kα radiation, wherein the 2Θ values are ±0.2 degrees 2Θ.

In another embodiment, Form C is characterized as having a XRPD pattern with peaks at 9.2, 19.8, and 29.6 degrees 2Θ using Cu Kα radiation, wherein the 2Θ values are ±0.2 degrees 2Θ.

In another embodiment, Form C is characterized as having a XRPD pattern with at least three peaks at 4.0, 6.2, 7.7, 8.9, 9.2, 9.9, 10.6, 12.7, 15.4, 16.5, 16.9, 18.4, 19.1, 19.4, 19.8, 20.4, 21.0, 21.3, 21.6, 22.1, 23.0, 23.3, 23.8, 24.6, 24.9, 25.4, 25.6, 25.9, 26.4, 27.3, 28.0, 28.8, 29.6, 30.1, 30.7, 30.9, 31.2, 31.5, 31.9, 32.6, 33.0, 33.5, 33.8, 34.2, 34.5, 34.8, 35.3, 35.6, 36.1, 36.4, 37.1, 37.7, 38.2, 38.7, 38.9, 39.3, and/or 39.8 degrees 2Θ using Cu Kα radiation, wherein the 2Θ values are ±0.2 degrees 2Θ.

In another embodiment, Form C is characterized as having a XRPD pattern with at least four peaks at 4.0, 6.2, 7.7, 8.9, 9.2, 9.9, 10.6, 12.7, 15.4, 16.5, 16.9, 18.4, 19.1, 19.4, 19.8, 20.4, 21.0, 21.3, 21.6, 22.1, 23.0, 23.3, 23.8, 24.6, 24.9, 25.4, 25.6, 25.9, 26.4, 27.3, 28.0, 28.8, 29.6, 30.1, 30.7, 30.9, 31.2, 31.5, 31.9, 32.6, 33.0, 33.5, 33.8, 34.2, 34.5, 34.8, 35.3, 35.6, 36.1, 36.4, 37.1, 37.7, 38.2, 38.7, 38.9, 39.3, and/or 39.8 degrees 2Θ using Cu Kα radiation, wherein the 2Θ values are ±0.2 degrees 2Θ.

In another embodiment, Form C is characterized as having a XRPD pattern with at least five peaks at 4.0, 6.2, 7.7, 8.9, 9.2, 9.9, 10.6, 12.7, 15.4, 16.5, 16.9, 18.4, 19.1, 19.4, 19.8, 20.4, 21.0, 21.3, 21.6, 22.1, 23.0, 23.3, 23.8, 24.6, 24.9, 25.4, 25.6, 25.9, 26.4, 27.3, 28.0, 28.8, 29.6, 30.1, 30.7, 30.9, 31.2, 31.5, 31.9, 32.6, 33.0, 33.5, 33.8, 34.2, 34.5, 34.8, 35.3, 35.6, 36.1, 36.4, 37.1, 37.7, 38.2, 38.7, 38.9, 39.3, and/or 39.8 degrees 2Θ using Cu Kα radiation, wherein the 2Θ values are ±0.2 degrees 2Θ.

In another embodiment, Form C is characterized as having a XRPD pattern with at least six peaks at 4.0, 6.2, 7.7, 8.9, 9.2, 9.9, 10.6, 12.7, 15.4, 16.5, 16.9, 18.4, 19.1, 19.4, 19.8, 20.4, 21.0, 21.3, 21.6, 22.1, 23.0, 23.3, 23.8, 24.6, 24.9, 25.4, 25.6, 25.9, 26.4, 27.3, 28.0, 28.8, 29.6, 30.1, 30.7, 30.9, 31.2, 31.5, 31.9, 32.6, 33.0, 33.5, 33.8, 34.2, 34.5, 34.8, 35.3, 35.6, 36.1, 36.4, 37.1, 37.7, 38.2, 38.7, 38.9, 39.3, and/or 39.8 degrees 2Θ using Cu Kα radiation, wherein the 2Θ values are ±0.2 degrees 2Θ.

In another embodiment, Form C is characterized as having a XRPD pattern with at least seven peaks at 4.0, 6.2, 7.7, 8.9, 9.2, 9.9, 10.6, 12.7, 15.4, 16.5, 16.9, 18.4, 19.1, 19.4, 19.8, 20.4, 21.0, 21.3, 21.6, 22.1, 23.0, 23.3, 23.8, 24.6, 24.9, 25.4, 25.6, 25.9, 26.4, 27.3, 28.0, 28.8, 29.6, 30.1, 30.7, 30.9, 31.2, 31.5, 31.9, 32.6, 33.0, 33.5, 33.8, 34.2, 34.5, 34.8, 35.3, 35.6, 36.1, 36.4, 37.1, 37.7, 38.2, 38.7, 38.9, 39.3, and/or 39.8 degrees 2Θ using Cu Kα radiation, wherein the 2Θ values are ±0.2 degrees 2Θ.

In another embodiment, Form C is characterized as having a XRPD pattern with at least eight peaks at 4.0, 6.2, 7.7, 8.9, 9.2, 9.9, 10.6, 12.7, 15.4, 16.5, 16.9, 18.4, 19.1, 19.4, 19.8, 20.4, 21.0, 21.3, 21.6, 22.1, 23.0, 23.3, 23.8, 24.6, 24.9, 25.4, 25.6, 25.9, 26.4, 27.3, 28.0, 28.8, 29.6, 30.1, 30.7, 30.9, 31.2, 31.5, 31.9, 32.6, 33.0, 33.5, 33.8, 34.2, 34.5, 34.8, 35.3, 35.6, 36.1, 36.4, 37.1, 37.7, 38.2, 38.7, 38.9, 39.3, and/or 39.8 degrees 2Θ using Cu Kα radiation, wherein the 2Θ values are ±0.2 degrees 2Θ.

In another embodiment, Form C is characterized as having a XRPD pattern with at least nine peaks at 4.0, 6.2, 7.7, 8.9, 9.2, 9.9, 10.6, 12.7, 15.4, 16.5, 16.9, 18.4, 19.1, 19.4, 19.8, 20.4, 21.0, 21.3, 21.6, 22.1, 23.0, 23.3, 23.8, 24.6, 24.9, 25.4, 25.6, 25.9, 26.4, 27.3, 28.0, 28.8, 29.6, 30.1, 30.7, 30.9, 31.2, 31.5, 31.9, 32.6, 33.0, 33.5, 33.8, 34.2, 34.5, 34.8, 35.3, 35.6, 36.1, 36.4, 37.1, 37.7, 38.2, 38.7, 38.9, 39.3, and/or 39.8 degrees 2Θ using Cu Kα radiation, wherein the 2Θ values are ±0.2 degrees 2Θ.

In another embodiment, Form C is characterized as having a XRPD pattern with at least ten peaks at 4.0, 6.2, 7.7, 8.9, 9.2, 9.9, 10.6, 12.7, 15.4, 16.5, 16.9, 18.4, 19.1, 19.4, 19.8, 20.4, 21.0, 21.3, 21.6, 22.1, 23.0, 23.3, 23.8, 24.6, 24.9, 25.4, 25.6, 25.9, 26.4, 27.3, 28.0, 28.8, 29.6, 30.1, 30.7, 30.9, 31.2, 31.5, 31.9, 32.6, 33.0, 33.5, 33.8, 34.2, 34.5, 34.8, 35.3, 35.6, 36.1, 36.4, 37.1, 37.7, 38.2, 38.7, 38.9, 39.3, and/or 39.8 degrees 2Θ using Cu Kα radiation, wherein the 2Θ values are ±0.2 degrees 2Θ.

In another embodiment, Form C is characterized as having a XRPD pattern with peaks at 4.0, 6.2, 7.7, 8.9, 9.2, 9.9, 10.6, 12.7, 15.4, 16.5, 16.9, 18.4, 19.1, 19.4, 19.8, 20.4, 21.0, 21.3, 21.6, 22.1, 23.0, 23.3, 23.8, 24.6, 24.9, 25.4, 25.6, 25.9, 26.4, 27.3, 28.0, 28.8, 29.6, 30.1, 30.7, 30.9, 31.2, 31.5, 31.9, 32.6, 33.0, 33.5, 33.8, 34.2, 34.5, 34.8, 35.3, 35.6, 36.1, 36.4, 37.1, 37.7, 38.2, 38.7, 38.9, 39.3, and/or 39.8 degrees 2Θ using Cu Kα radiation, wherein the 2Θ values are ±0.2 degrees 2Θ.

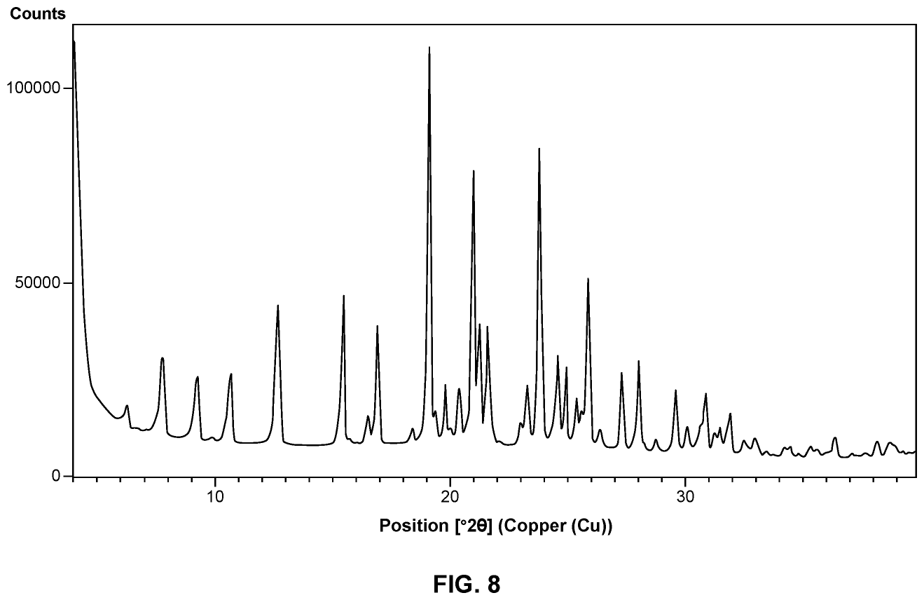

In another embodiment, Form C is characterized as having a XRPD difractogram that is essentially the same as the one depicted in FIG. 8.

In another embodiment, Form C is characterized as having a FT-Raman spectrum with peaks at 3056, 2960, 2992, 1735, 1608, 1205, 824, 656, 170 cm⁻¹, wherein the cm⁻¹ values are ±4 cm⁻¹.

In another embodiment, Form C, characterized as having an FT-Raman spectrum with peaks at 3056, 3017, 2992, 2960, 2921, 1735, 1608, 1582, 1472, 1437, 1388, 1333, 1314, 1295, 1246, 1205, 1184, 1147, 1072, 1014, 928, 901, 842, 824, 772, 736, 719, 656, 646, 636, 601, 531, 511, 467, 440, 397, 329, 274, and 170 cm⁻¹, wherein the cm⁻¹ values are ±4 cm⁻¹.

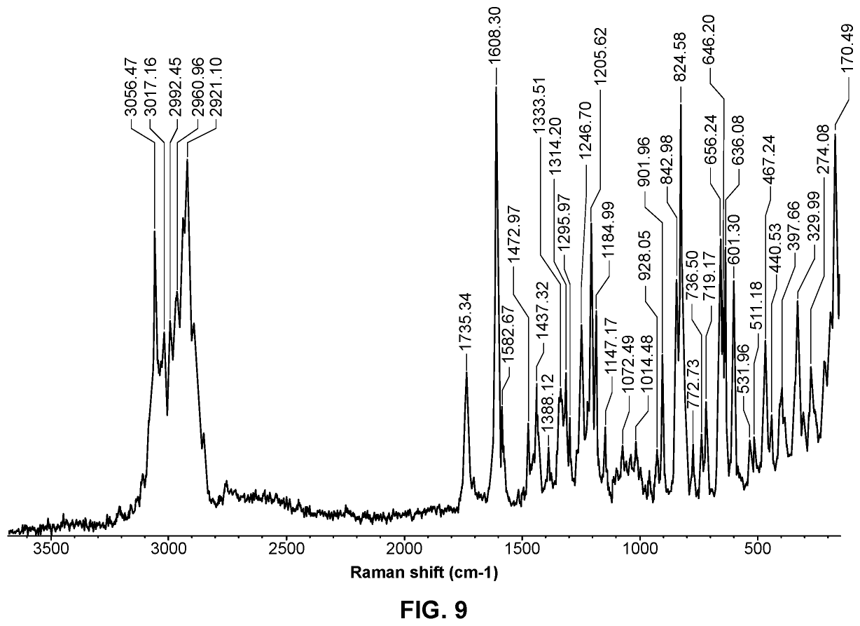

In another embodiment, Form C is characterized as having a FT-Raman spectrum that is essentially the same as the one depicted in FIG. 9.

In another embodiment, Form C is characterized as having a melting point with an onset temperature of about 149.45° C. and a peak temperature of about 153.50° C. based on differential scanning calorimetry.

In another embodiment, Form C is characterized as having a first broad endotherm with an onset temperature of about 32.74° C. and a peak temperature of about 66.50° C., and a second sharp endotherm with an onset temperature of about 149.45° C. and a peak temperature of about 153.50° C. based on differential scanning calorimetry.

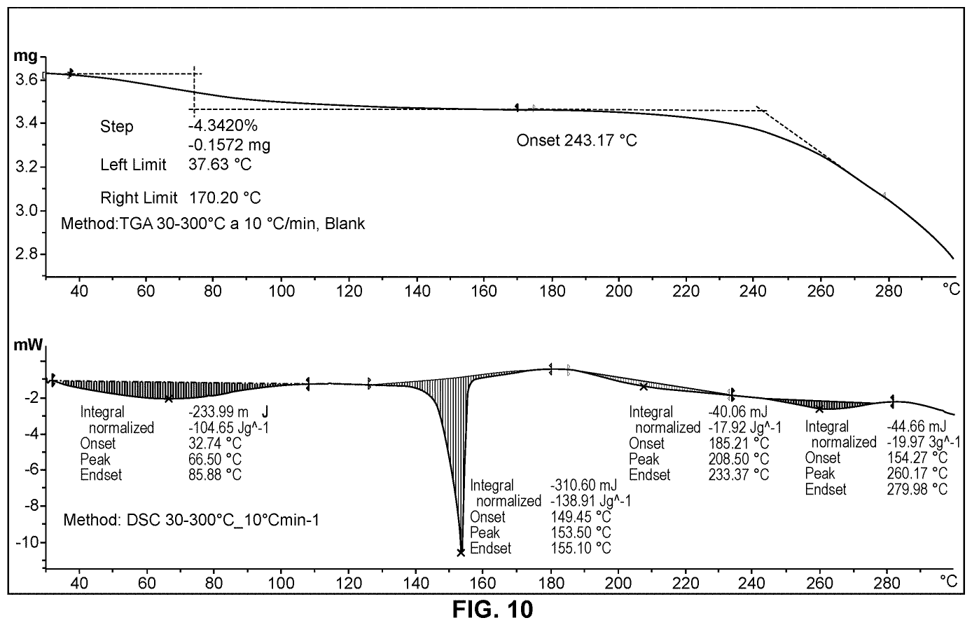

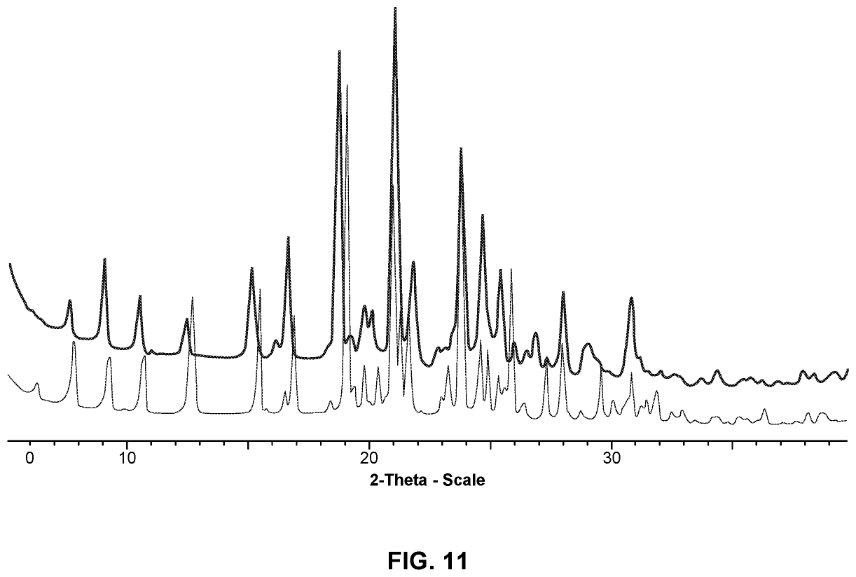

In another embodiment, Form C is characterized as having a DSC thermogram that is essentially the same as the one depicted in FIG. 10.

In another embodiment, Form C is characterized as having a TGA plot that is essentially the same as the one depicted in FIG. 10.

In another embodiment, Form C is characterized as having a TGA comprising a mass loss of about 5.0%, e.g., about 4.8%, e.g., about 4.6%, e.g., about 4.3%, e.g., about 4.0%, e.g., about 3.80%, of the total mass of the sample upon heating from about 40° C. to about 160° C. In another embodiment, Form C contains water in the crystal lattice or water admixed with other organic solvent in the crystal lattice. In certain embodiments, Form C is a hydrate represented by the formula: leriglitazone·xH₂O, wherein x is 0.5 to 3. In certain embodiments, Form C is a monohydrate, e.g., x is about 1.

In another embodiment, Form C is characterized by its stability profile. In another embodiment, Form C material is stable, e.g., its XRPD pattern remains substantially unchanged, upon exposure to elevated humidity, upon exposure to one or more solvents, and/or upon compression.

In another embodiment, the present disclosure provides a pharmaceutical composition comprising a suspension of a leriglitazone polymorph, e.g., leriglitazone hydrate Form C, in water and, optionally, one or more pharmaceutically acceptable carriers, diluents or excipients.

In another embodiment, the present disclosure provides a pharmaceutical formulation comprising a suspension of leriglitazone hydrate Form C in water and, optionally, one or more one or more pharmaceutically acceptable excipients.

### IV. METHODS OF MAKING CRYSTALLINE POLYMORPHIC FORMS OF LERIGLITAZONE

In another embodiment, the invention provides a method of making crystalline Form A of leriglitazone as defined above, comprising the following steps:
(a) providing leriglitazone hydrochloride salt, in an organic solvent or a mixture of organic solvents to provide a solution or a suspension,
(b) optionally, heating the mixture of step (a) at a suitable temperature, preferably from 40° C. to 100° C., more preferably from 50° C. to 75° C.,
(c) optionally, cooling the solution or the suspension resulting from step (b) to room temperature, and
(d) isolating crystalline Form A of leriglitazone hydrochloride.

In another embodiment, the invention provides a method of making crystalline Form B of leriglitazone as defined above, comprising the following steps:
(a) providing leriglitazone hydrochloride salt, in water or in mixtures of water with an alcohol solvent to provide a solution or a suspension,
(b) optionally, heating the mixture of step (a) at a suitable temperature, preferably from 40° C. to 100° C., more preferably from 50° C. to 85° C.,
(c) optionally, cooling the solution or the suspension resulting from step (b) to room temperature, and
(d) isolating and drying the solid obtained in step (c) providing Form B of leriglitazone.

In an embodiment, drying of step (d) may be carried out at atmospheric pressure or under reduced pressure and at room temperature or at temperatures from 50° C. to 130° C., preferably from 80° C. to 110° C. In an embodiment, drying of step (d) is carried out at under reduced pressure and at temperatures from 50° C. to 130° C., preferably from 80° C. to 110° C.

In another embodiment, Form B may be prepared by drying leriglitazone hydrate Form C under reduced pressure at temperatures from 50° C. to 130° C.

In another embodiment, the invention provides a method of making crystalline leriglitazone hydrate Form C as defined above, comprising the following steps:
(a) providing leriglitazone hydrochloride salt, in water or in mixtures of water with an alcohol solvent to provide a solution or a suspension,
(b) optionally, heating the mixture of step (a) at a suitable temperature, preferably from 40° C. to 100° C., more preferably from 50° C. to 85° C.,
(c) optionally, cooling the solution or the suspension resulting from step (b) to room temperature, and
(d) isolating the solid obtained in step (c) providing Form C of leriglitazone.

Leriglitazone hydrochloride used in (a) may be in solvate, hydrate, anhydrous, crystalline form, or non-crystalline form thereof. Preferably, the preparation of either crystalline Form B or hydrate Form C starts in step (a) from leriglitazone hydrochloride Form A.

### V. PHARMACEUTICAL COMPOSITIONS

The pharmaceutical compositions disclosed herein contain conventional excipients known in the art and may be prepared by conventional methods. Oral dosage forms may be prepared by combining leriglitazone, or a pharmaceutically acceptable salt thereof, e.g., Form A, in an intimate admixture with at least one excipient, e.g., water, according to conventional pharmaceutical compounding techniques.

For example, leriglitazone, or a pharmaceutically acceptable salt or hydrate thereof, may be suspended in a pharmaceutically acceptable liquid carrier such as water. The liquid carrier may contain other suitable pharmaceutical additives including, but not limited to, solubilizers, emulsifiers, buffers, preservatives, sweeteners, flavouring agents, suspending agents, thickening agents, colouring agents, viscosity regulators, stabilizers, pH regulators, or osmo-regulators. Suitable examples of liquid carriers for oral administration include water optionally containing additives as above, e.g. cellulose derivatives, such as sodium carboxymethyl cellulose solution. In one embodiment, the present disclosure provides a pharmaceutical formulation comprising leriglitazone, a pharmaceutically acceptable salt thereof, or a hydrate thereof, in an amount from 0.01% w/v to 5% w/v and quantum satis (QS) water to 100%.

In another embodiment, the present disclosure provides a pharmaceutical formulation comprising 0.01% w/v to 5% w/v of leriglitazone hydrate Form C and QS water to 100%, e.g., about 1.43 g of Form C in 100 ml of water.

A pharmaceutical composition comprising leriglitazone, or a pharmaceutically acceptable salt or hydrate thereof, may be provided as a kit in a suitable container, e.g., a multi-dose Type III amber glass bottle, as primary packaging and packaged in a carton box as secondary packaging and, optionally, a label. A graduated oral syringe of 5 mL, 10 mL, 12 mL, or 15 mL may also be provided for dosing purposes.

The present disclosure provides the following particular embodiments with respect to pharmaceutical compositions.

### Embodiment 1F

A pharmaceutical formulation comprising: (a) leriglitazone, a pharmaceutically acceptable salt thereof, or a hydrate thereof, in an amount from 0.01% w/v to 5% w/v; (b) one or more cellulose derivatives in an amount from 0.01% w/v to 2% w/v; and (c) water QS to 100%.

### Embodiment 2F

The pharmaceutical formulation according to Embodiment 1F, wherein the leriglitazone, the pharmaceutically acceptable salt thereof, or the hydrate thereof, is leriglitazone hydrate Form C.

### Embodiment 3F

The pharmaceutical formulation according to Embodiments 1F or 2F, wherein the one or more cellulose derivatives comprise microcrystalline cellulose, carboxymethylcellulose sodium, or mixtures thereof.

### Embodiment 4F

The pharmaceutical formulation according to any one of Embodiments 1F-3F further comprising one or more pH regulators.

### Embodiment 5F

The pharmaceutical formulation according to Embodiment 4F, wherein the one or more pH regulators comprise sodium citrate, citric acid monohydrate, or mixtures thereof.

### Embodiment 6F

The pharmaceutical formulation according to any one of Embodiments 1F-5F further comprising one or more preservatives in an amount from 0.001% w/v to 1.0% w/v.

### Embodiment 7F

The pharmaceutical formulation according to Embodiment 6F, wherein the one or more preservatives comprises sodium benzoate.

### Embodiment 8F

The pharmaceutical formulation according to any one of Embodiments 1F-7F further comprising one or more flavouring agents in an amount of 0.001% w/v to 3% w/v.

### Embodiment 9F

The pharmaceutical formulation according to Embodiment 6F, wherein the one or more flavouring agents comprise strawberry flavor.

### Embodiment 10F

The pharmaceutical formulation according to any one of Embodiments 1F-9F further comprising one or more sweetening agents in an amount of 0.01% w/v to 15% w/v.

### Embodiment 11F

The pharmaceutical formulation according to Embodiment 10, wherein the sweetening agent is sorbitol, saccharin sodium, or mixtures thereof.

### Embodiment 12F

The pharmaceutical formulation according to any one of Embodiments 1F-11F, wherein the pH of the formulation is 3.5 to 4.5.

### Embodiment 13F

The pharmaceutical formulation according to Embodiment 12F, wherein the pH of the formulation is about 4.

### Embodiment 14F

The pharmaceutical formulation according to any one of Embodiments 1F-13F, wherein the viscosity of the formulation is higher than 50 mPa*s and less than 5000 mPa*s, e.g., less than 4500 mPa*s or less than 4000 mPa*s as measured following standard method described in the Ph. Eur. 2.2.8.

### Embodiment 15F

A pharmaceutical formulation comprising leriglitazone hydrate Form C prepared by admixing: (a) leriglitazone hydrochloride Form A; and (b) water.

### Embodiment 16F

The pharmaceutical formulation of Embodiment 15F prepared by admixing (a) about 1.5% w/v leriglitazone hydrochloride Form A; (b) about 8% w/v sorbitol powder; (c) about 1% w/v microcrystalline cellulose; (d) about 0.5% w/v carboxymethyl cellulose sodium; (e) about 0.05% w/v saccharin sodium; (f) about 0.1% w/v sodium benzoate; (g) about 0.5% w/v sodium citrate; (h) about 0.1% w/v citric acid monohydrate; (i) about 0.015% w/v strawberry flavour; (j) about 1% w/v 0.2 M aqueous citric acid monohydrate solution; and (k) water QS to 100%.

### Embodiment 17F

A pharmaceutical formulation comprising (a) leriglitazone, a pharmaceutically acceptable salt, or a hydrate thereof, preferably hydrate Form C; in an amount of 1.0% to 2.0% w/v; (b) about 1% w/v of microcrystalline cellulose; (c) about 0.5% w/v of carboxymethyl cellulose sodium; (d) water QS to 100%; and, optionally; (e) about 8% w/v sorbitol powder; (f) about 0.05% w/v saccharin sodium; (g) about 0.1% w/v sodium benzoate; (h) about 0.5% w/v sodium citrate; (i) about 0.1% w/v citric acid monohydrate; (j) about 0.015% w/v strawberry flavour; (k) about 1% w/v 0.2 M aqueous citric acid monohydrate solution.

### Embodiment 18F

A pharmaceutical formulation comprising (a) about 1.43% w/v leriglitazone hydrate, e.g., Form C; (b) about 8% w/v sorbitol powder; (c) about 1% w/v microcrystalline cellulose; (d) about 0.5% w/v carboxymethyl cellulose sodium; (e) about 0.05% w/v saccharin sodium; (f) about 0.1% w/v sodium benzoate; (g) about 0.5% w/v sodium citrate; (h) about 0.1% w/v citric acid monohydrate; (i) about 0.015% w/v strawberry flavour; (j) about 1% w/v 0.2 M aqueous citric acid monohydrate solution; and (k) water QS to 100%.

### Embodiment 19F

The pharmaceutical composition of any one of Embodiments 1F-14F, wherein the cellulose derivative has a viscosity between 1500 to 4500 mPa*s, e.g., between 1500 to 3500 mPa*s, e.g., between 1500 to 3000 mPa*s, as measured by Brookfield viscosity method.

Advantageously, the pharmaceutical formulation according to any one of Embodiments 1F-19F allows a variable and patient-adapted dose of leriglitazone. Additionally, the leriglitazone suspension formulation of the present disclosure has at rest, a thixotropic polymeric gel structure, in other words, its viscosity can only be observed once fluidified by gentle manual shaking just prior administration. The viscosity is broken down by agitation during shaking but then re-forms upon aging. The apparent viscosity of thixotropic suspensions is therefore dependent on their previous shear history, including the duration of the shearing. This property is useful for suspensions because the structure formed on standing prevents sedimentation. Therefore, leriglitazone suspension formulation of the present disclosure is thixotropic and no sediments are observed at rest, thereby the need of re-dispersibility is avoided.

### VI. Methods of Treating a Disease, Disorder, or Condition

In another embodiment, the present disclosure provides a leriglitazone polymorph or a pharmaceutical composition or formulation comprising a leriglitazone polymorph for use in treating a disease or disorder in a patient in need thereof, wherein the disease or disorder is a central nervous system disease or disorder, mitochondrial disease, a liver disease or disorder, chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, an inflammatory respiratory disease, or a lung disease or disorder.

In another embodiment, the disease or disorder is a central nervous system disease or disorder.

In another embodiment, the central nervous system disease or disorder is selected from the group consisting of neurodegenerative disease, cerebrovascular disease, seizure, epilepsy, viral disease, neuroinflammatory disease, brain tumor, organic acidemias, fatty acid disorder, and genetic mitochondrial disorder.

In another embodiment, the central nervous system disease or disorder is a neurodegenerative disease.

In another embodiment, the neurodegenerative disease is selected from the group consisting of Alzheimer's disease, Huntington's chorea, Parkinson's disease, multiple sclerosis, neuromyelitis optica, leukodystrophy, amyotrophic lateral sclerosis (ALS), degenerative ataxia, multiple system atrophy, neurodegeneration and brain iron accumulation disorders (NBIA), neuromyopathy, and a motor neuron disease.

In another embodiment, the leukodystrophy is X-linked adrenoleukodystrophy, adrenomyeloneuropathy, cerebral adrenoleukodystrophy, or metachromatic leukodystrophy.

In another embodiment, the leukodystrophy is cerebral adrenoleukodystrophy.

In another embodiment, the degenerative ataxia is Friedreich's ataxia.

In another embodiment, the motor neuron disease is selected from the group consisting of progressive bulbar palsy, pseudobulbar palsy, primary lateral sclerosis (PLS), progressive muscular atrophy, spinal muscular atrophy (SMA), post-polio syndrome (PPS)-Marie-Tooth disease, Guillan-Barré syndrome, and adrenomyeloneuropathy (AMN).

In another embodiment, the cerebrovascular disease is selected from the group consisting of global or local ischemia, intracerebral haemorrhage, stroke, and vascular dementia.

In another embodiment, the viral disease is selected from the group consisting of meningitis, encephalitis, rabies, measles, mumps, poliomyelitis, herpes simplex, and varicella zoster.

In another embodiment, the central nervous system disease or disorder is a rare metabolic disease selected from the group consisting of organic acidemias, fatty acid disorders and genetic mitochondrial disorders.

In another embodiment, the disease or disorder is a mitochondrial disease.

In another embodiment, the mitochondrial disease is a primary mitochondrial disorder selected from the group consisting of Rett syndrome, Alper's disease; Leber's hereditary optic neuropathy (LHON); Kearns-Sayre syndrome (KSS); Leigh's syndrome; Leigh-like syndrome; maternally inherited Leigh syndrome (MILS); mitochondrial depletion syndrome (MDS); mitochondrial DNA depletion syndrome (MDDS); mitochondrial encephalomyopathy; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; chronic progressive external opthalmoplegia (CPEO); dominant optic atrophy (DOA); autosomal dominant optic atrophy (ADOA); mitochondrial myopathy; cardiomyopathy; mitochondrial encephalopathy; myoclonic epilepsy; maternally inherited diabetes and deafness (MIDD); ataxia neuropathy spectrum; 3-methylglutaconic aciduria; sensoneural deafness; neuroradiological findings of Leigh-like syndrome (MEGDEL); SURF1 (COX definient Leigh syndrome due to complex IV surfeit protein deficiency); oxidative phosphorylation disorders; Berth syndrome; lethal infantile cardiomyopathy (LIC); pyruvate carboxylase deficiency; pyruvate dehydrogenase deficiency; POLG mutation; isolated or combined OXPHOS deficiencies with so far unsolved genetic defect including disturbed pyruvate oxidation and ATP plus PCr production rates; POLG2 mutation;

carnitine-acyl-cartinine deficiency; carnitine deficiency; creatinine deficiency syndromes; Co-Enzyme Q10 deficiency; Complex I deficiency; Complex II deficiency; Complex III deficiency; Complex IV deficiency; Complex V deficiency; lactic acidosis; leukoencephalopathy with brain stem and spinal cord involvement and lactate elevation (LBSL); Luft disease; carnitine palmitoyltransferase (CPT I or CPT II) deficiency; short-chain acyl-CoA dehydrogenase deficiency (SCAD); short-chain 3-hydroxyacetyl-CoA dehydrogenase deficiency (SCHAD); medium-chain acyl-CoA dehydrogenase deficiency (MCAD); multiple acyl-CoA dehydrogenase deficiency (MADD); long-chain acyl-CoA dehydrogenase deficiency (LCAD); very long-chain acyl-CoA dehydrogenase deficiency (VLCAD); trifunctional protein (TFP) deficiency; and glutaric aciduria Type II.

In another embodiment, the mitochondrial disease is selected from the group consisting of Rett syndrome; dominant optic atrophy (DOA); autosomal dominant optic atrophy (ADOA); Complex I deficiency; Leber hereditary optic neuropathy (LHON); Kearns-Sayre syndrome (KSS); Leigh's syndrome; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; and chronic progressive external opthalmoplegia (CPEO).

In another embodiment, the mitochondrial disease is a secondary mitochondrial disorder selected from the group consisting of Duchenne muscular dystrophy (DMD); Becker muscular dystrophy (BMD); myotonic dystrophy (BMD); congenital myopathies; glycogen storage disorders; spinal-bulbar muscular atrophy (SBMA); argininosuccinic aciduria; autism spectrum disorder (ASD); autoimmune diseases of the skin (such as pemphigus vulgaris and lupus); methylmalonic and propionic acidurias; disorders or purine and/or pyrimidine synthesis; facioscapulohumeral muscular dystrophy (FSHD); congenital muscular dystrophies; collagen VI muscular dystrophies (e.g., Ullrich congenital muscular dystrophy, Bethlem myopathy, oculopharyngeal distal and Emery-Dreifuss); DiGeorge syndrome; and neuromuscular disorders (such as limb-girdle muscular dystrophy, inflammatory myopathies, Charcot Marie Tooth (CMT) neuropathy, and drug-induced peripheral neuropathies).

In another embodiment, the lung disease or disorder is an inflammatory lung condition or disease caused by a viral infection, acute inflammation of the lung, or interstitial lung disease.

In another embodiment, the disclosure provides a method for treating an inflammatory lung condition or disease caused by a viral infection in a patient in need thereof.

In another embodiment, the viral infection is a human coronavirus infection, an influenza virus infection, or a HIV virus infection.

In another embodiment, the human coronavirus is HCOV 229E, HCOV OC43, HCOV NL63, HCOV HKU1, SARS COV, MERS CoV, or SARS COV-2, or a mutated strain thereof.

In another embodiment, the human coronavirus is SARS COV-2.

In another embodiment, the human coronavirus is a mutated strain of SARS CoV-2.

In another embodiment, the inflammatory lung condition or disease caused by the viral infection is hypercytokinaemia, haemophagocytic lymphohistiocytosis, pneumonia, acute respiratory distress syndrome, or systemic inflammatory response syndrome.

In another embodiment, the inflammatory lung condition or disease caused by the viral infection is acute respiratory distress syndrome.

In another embodiment, the present disclosure provides leriglitazone polymorph for treating acute inflammation of the lung in a patient in need thereof.

In another embodiment, the acute inflammation of the lung is caused by a bacterial infection.

In another embodiment, the acute inflammation of the lung is pneumonia or acute respiratory distress syndrome.

In another embodiment, the present disclosure provides leriglitazone polymorph for treating interstitial lung disease in a patient in need thereof.

In another embodiment, the interstitial lung disease is idiopathic pulmonary fibrosis.

In another embodiment, the liver disease or disorder is nonalcoholic steatohepatitis (NASH) or nonalcoholic fatty liver disease (NAFLD).

In another embodiment, the present disclosure provides a method of treating a disease or disorder in a patient in need thereof, the method comprising administering a therapeutically effective amount of the leriglitazone polymorph or a pharmaceutical composition of leriglitazone polymorph, to the patient, wherein the disease or disorder is a central nervous system disease or disorder, mitochondrial disease, a liver disease or disorder, chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, an inflammatory respiratory disease, or a lung disease or disorder.

In another embodiment, the disease or disorder is a central nervous system disease or disorder.

In another embodiment, the central nervous system disease or disorder is selected from the group consisting of neurodegenerative disease, cerebrovascular disease, seizure, epilepsy, viral disease, neuroinflammatory disease, brain tumor, organic acidemias, fatty acid disorder, and genetic mitochondrial disorder.

In another embodiment, the central nervous system disease or disorder is a neurodegenerative disease.

In another embodiment, the neurodegenerative disease is selected from the group consisting of Alzheimer's disease, Huntington's chorea, Parkinson's disease, multiple sclerosis, neuromyelitis optica, leukodystrophy, ALS, degenerative ataxia, multiple system atrophy, NBIA (neurodegeneration and brain iron accumulation disorders), neuromyopathy, and a motor neuron disease.

In another embodiment, the leukodystrophy is X-linked adrenoleukodystrophy, adrenomyeloneuropathy, cerebral adrenoleukodystrophy, or metachromatic leukodystrophy.

In another embodiment, the leukodystrophy is cerebral adrenoleukodystrophy.

In another embodiment, the degenerative ataxia is Friedreich's ataxia.

In another embodiment, the motor neuron disease is selected from the group consisting of progressive bulbar palsy, pseudobulbar palsy, primary lateral sclerosis (PLS), progressive muscular atrophy, spinal muscular atrophy (SMA), post-polio syndrome (PPS)-Marie-Tooth disease, Guillan-Barré syndrome, and adrenomyeloneuropathy (AMN).

In another embodiment, the central nervous system disorder is a cerebrovascular disease selected from the group consisting of global or local ischemia, intracerebral haemorrhage, stroke, and vascular dementia.

In another embodiment, the central nervous system disorder is a viral disease selected from the group consisting of meningitis, encephalitis, rabies, measles, mumps, poliomyelitis, herpes simplex, and varicella zoster.

In another embodiment, the central nervous system disease or disorder is a rare metabolic disease selected from the group consisting of organic acidemias, fatty acid disorders and genetic mitochondrial disorders.

In another embodiment, the disease or disorder is a mitochondrial disease.

In another embodiment, the mitochondrial disease is a primary mitochondrial disorder selected from the group consisting of Rett syndrome, Alper's disease; Leber's hereditary optic neuropathy (LHON); Kearns-Sayre syndrome (KSS); Leigh's syndrome; Leigh-like syndrome; maternally inherited Leigh syndrome (MILS); mitochondrial depletion syndrome (MDS); mitochondrial DNA depletion syndrome (MDDS); mitochondrial encephalomyopathy; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; chronic progressive external opthalmoplegia (CPEO); dominant optic atrophy (DOA); autosomal dominant optic atrophy (ADOA); mitochondrial myopathy; cardiomyopathy; mitochondrial encephalopathy; myoclonic epilepsy; maternally inherited diabetes and deafness (MIDD); ataxia neuropathy spectrum; 3-methylglutaconic aciduria; sensoneural deafness; neuroradiological findings of Leigh-like syndrome (MEGDEL); SURF1 (COX definient Leigh syndrome due to complex IV surfeit protein deficiency); oxidative phosphorylation disorders; Berth syndrome; lethal infantile cardiomyopathy (LIC); pyruvate carboxylase deficiency; pyruvate dehydrogenase deficiency; POLG mutation; isolated or combined OXPHOS deficiencies with so far unsolved genetic defect including disturbed pyruvate oxidation and ATP plus PCr production rates; POLG2 mutation; carnitine-acyl-cartinine deficiency; carnitine deficiency; creatinine deficiency syndromes; Co-Enzyme Q10 deficiency; Complex I deficiency; Complex II deficiency; Complex III deficiency; Complex IV deficiency; Complex V deficiency; lactic acidosis; leukoencephalopathy with brain stem and spinal cord involvement and lactate elevation (LBSL); Luft disease; carnitine palmitoyltransferase (CPT I or CPT II) deficiency; short-chain acyl-CoA dehydrogenase deficiency (SCAD); short-chain 3-hydroxyacetyl-CoA dehydrogenase deficiency (SCHAD); medium-chain acyl-CoA dehydrogenase deficiency (MCAD); multiple acyl-CoA dehydrogenase deficiency (MADD); long-chain acyl-CoA dehydrogenase deficiency (LCAD); very long-chain acyl-CoA dehydrogenase deficiency (VLCAD); trifunctional protein (TFP) deficiency; and glutaric aciduria Type II.

In another embodiment, the mitochondrial disease is selected from the group consisting of Rett syndrome; dominant optic atrophy (DOA); autosomal dominant optic atrophy (ADOA); Complex I deficiency; Leber hereditary optic neuropathy (LHON); Kearns-Sayre syndrome (KSS); Leigh's syndrome; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; and chronic progressive external opthalmoplegia (CPEO).

In another embodiment, the mitochondrial disease is a secondary mitochondrial disorder selected from the group consisting of Duchenne muscular dystrophy (DMD); Becker muscular dystrophy (BMD); myotonic dystrophy (BMD); congenital myopathies; glycogen storage disorders; spinal-bulbar muscular atrophy (SBMA); argininosuccinic aciduria; autism spectrum disorder (ASD); autoimmune diseases of the skin (such as pemphigus vulgaris and lupus); methylmalonic and propionic acidurias; disorders or purine and/or pyrimidine synthesis; facioscapulohumeral muscular dystrophy (FSHD); congenital muscular dystrophies; collagen VI muscular dystrophies (e.g., Ullrich congenital muscular dystrophy, Bethlem myopathy, oculopharyngeal distal and Emery-Dreifuss); DiGeorge syndrome; and neuromuscular disorders (such as limb-girdle muscular dystrophy, inflammatory myopathies, Charcot Marie Tooth (CMT) neuropathy, and drug-induced peripheral neuropathies).

In another embodiment, the lung disease or disorder is an inflammatory lung condition or disease caused by a viral infection, acute inflammation of the lung, or interstitial lung disease.

In another embodiment, the present disclosure provides leriglitazone polymorph for treating an inflammatory lung condition or disease caused by a viral infection in a patient in need thereof.

In another embodiment, the viral infection is a human coronavirus infection, an influenza virus infection, or a HIV virus infection.

In another embodiment, the human coronavirus is HCOV 229E, HCoV OC43, HCOV NL63, HCOV HKU1, SARS COV, MERS CoV, or SARS COV-2, or a mutated strain thereof.

In another embodiment, the human coronavirus is SARS COV-2.

In another embodiment, the human coronavirus is a mutated strain of SARS CoV-2.

In another embodiment, the inflammatory lung condition or disease caused by the viral infection is hypercytokinaemia, haemophagocytic lymphohistiocytosis, pneumonia, acute respiratory distress syndrome, or systemic inflammatory response syndrome.

In another embodiment, the inflammatory lung condition or disease caused by the viral infection is acute respiratory distress syndrome.

In another embodiment, the present disclosure provides leriglitazone polymorph for treating acute inflammation of the lung in a patient in need thereof.

In another embodiment, the acute inflammation of the lung is caused by a bacterial infection.

In another embodiment, the acute inflammation of the lung is pneumonia or acute respiratory distress syndrome.

In another embodiment, the present disclosure provides leriglitazone polymorph for treating interstitial lung disease in a patient in need thereof.

In another embodiment, the interstitial lung disease is idiopathic pulmonary fibrosis.

In another embodiment, the liver disease or disorder is nonalcoholic steatohepatitis (NASH) or nonalcoholic fatty liver disease (NAFLD).

### VII. Compounds and Compositions

In another embodiment, the present disclosure provides compositions comprising leriglitazone (free base), leriglitazone hydrochloride, or leriglitazone hydrate, e.g., Form A, Form B, or Form C, and (i) (Z)-5-(4-(2-(5-(1-hydroxyethyl)pyridin-2-yl)ethoxy)benzylidene)thiazolidine-2,4-dione, or the hydrochloride salt thereof; (ii) 2,2′-disulfanediylbis(3-(4-(2-(5-(1-hydroxyethyl)pyridin-2-yl)ethoxy)phenyl) propanoic acid), or the hydrochloride salt thereof; (iii) 2-((1-hydroxy-3-(4-(2-(5-(1-hydroxyethyl)pyridin-2-yl)ethoxy)phenyl) propan-2-yl)disulfaneyl)-3-(4-(2-(5-(1-hydroxyethyl)pyridin-2-yl)ethoxy)phenyl) propanoic acid, or the hydrochloride salt thereof; and/or (iv) 5-(4-(2-(5-(1-hydroxyethyl)pyridin-2-yl)ethoxy)benzyl) thiazolidin-2-one, or the hydrochloride salt thereof, as impurities that are unexpectedly produced during the leriglitazone manufacturing process.

In another embodiment, the present disclosure provides a composition comprising 99.0 to 99.9 wt/wt % of leriglitazone (free base), leriglitazone hydrochloride, or leriglitazone hydrate, e.g., Form A, Form B, or Form C, and: (i) 0.0001 to 0.2 wt/wt % of (Z)-5-(4-(2-(5-(1-hydroxyethyl)pyridin-2-yl)ethoxy)benzylidene)thiazolidine-2,4-dione, or the hydrochloride salt thereof; (ii) 0.0001 to 0.2 wt/wt % of 2,2′-disulfanediylbis(3-(4-(2-(5-(1-hydroxyethyl)pyridin-2-yl)ethoxy)phenyl) propanoic acid), or the hydrochloride salt thereof; (iii) 0.0001 to 0.2 wt/wt % of 2-((1-hydroxy-3-(4-(2-(5-(1-hydroxyethyl)pyridin-2-yl)ethoxy)phenyl) propan-2-yl)disulfaneyl)-3-(4-(2-(5-(1-hydroxyethyl)pyridin-2-yl)ethoxy)phenyl) propanoic acid, or the hydrochloride salt thereof; and/or (iv) 0.0001 to 0.15 wt/wt % of 5-(4-(2-(5-(1-hydroxyethyl)pyridin-2-yl)ethoxy)benzyl) thiazolidin-2-one, or the hydrochloride salt thereof.

In another embodiment, the present disclosure provides (Z)-5-(4-(2-(5-(1-hydroxyethyl)pyridin-2-yl)ethoxy)benzylidene)thiazolidine-2,4-dione, or a pharmaceutically acceptable salt thereof.

In another embodiment, the present disclosure provides 2,2′-disulfanediylbis(3-(4-(2-(5-(1-hydroxyethyl)pyridin-2-yl)ethoxy)phenyl) propanoic acid), or a pharmaceutically acceptable salt thereof.

In another embodiment, the present disclosure provides 2-((1-hydroxy-3-(4-(2-(5-(1-hydroxyethyl)pyridin-2-yl)ethoxy)phenyl) propan-2-yl)disulfaneyl)-3-(4-(2-(5-(1-hydroxyethyl)pyridin-2-yl)ethoxy)phenyl) propanoic acid, or a pharmaceutically acceptable salt thereof.

In another embodiment, the present disclosure provides 5-(4-(2-(5-(1-hydroxyethyl)pyridin-2-yl)ethoxy)benzyl) thiazolidin-2-one, or a pharmaceutically acceptable salt thereof.

### VIII. Definitions

The term “leriglitazone” as used herein refers to a compound having Formula I:

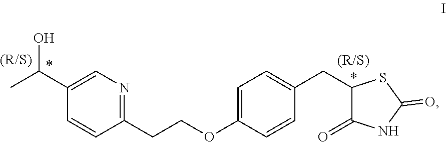 [SMILES: CC(O)c1ccc(CCOc2ccc(CC3SC(=O)NC3=O)cc2)nc1]

wherein each stereocenter is either in an R or S configuration. Leriglitazone is a mixture of four possible stereoisomers. The IUPAC name of leriglitazone is 5-[[4-[2-[5-(1-hydroxyethyl)pyridin-2-yl]ethoxy]phenyl]methyl]-1,3-thiazolidine-2,4-dione.

Leriglitazone may form crystalline solids that incorporate water into the crystal lattice without chemical alteration of the leriglitazone molecule to give, for example, a leriglitazone hydrate, e.g., Form C.

As used herein, the term “hydrate” refers to a crystalline form of a molecule that further comprises water incorporated into the crystalline structure. The water molecules in the hydrate may be present in a regular arrangement and/or a non-ordered arrangement. The hydrate may comprise either a stoichiometric or a nonstoichiometric amount of the water molecules.

As used herein, the term “solvate” refers to a crystalline form of a molecule that further comprises solvent molecule/s incorporated into the crystalline structure. When the solvent incorporated in the crystal is water, is called hydrate. The solvent molecules in the solvate may be present in a regular arrangement and/or a non-ordered arrangement. The solvate may comprise either a stoichiometric or a nonstoichiometric amount of the solvent molecules. Solvates can exhibit polymorphism.

As used herein, the term “organic solvent” refers to an organic molecule capable of at least partially dissolving another substance (i.e., the solute). Solvents may be liquids at room temperature. Suitable solvents may be, but are not limited to (C₆-C₁₄) aromatic hydrocarbon solvents such as toluene, o-xylene, m-xylene, and p-xylene; halogenated (C₁-C₁₂) hydrocarbon solvents such as 1,2-dichloroethane, dichloromethane, chloroform; (C₁-C₁₂) ether solvents such as diethyl ether, dipropyl ether, diphenyl ether, isopropyl ether, tert-butyl methyl ether, tetrahydrofuran, 1,4-dioxane; (C₁-C₁₂) ester solvents such as ethyl formate, methyl acetate, ethyl acetate, isopropyl acetate, butyl acetate, isobutyl acetate, ethyl malonate; (C₃-C₁₂) ketone solvents such as acetone, methyl ethyl ketone or 2-butanone, methyl isobutyl ketone, cyclohexanone, cyclopentanone, 3-pentanon; (C₁-C₁₂) alcohol solvents such as methanol, ethanol, isopropanol, 1-propanol, 2-methyl-1-propanol, 1-butanol, 2-butanol, 1-pentanol, 3-methyl-1-butanol, tert-butanol, 1-octanol, benzylalcohol, phenol, trifluoroethanol, glycerol, ethylene glycol, propylene glycol, m-cresol; nitrobenzene; N,N-dimethylformamide; N,N-dimethylacetamide; N-methyl-2-pyrrolidone; or acetonitrile. In another embodiment, the solvent is formed by the combination of two or more organic solvents.

As used herein, the term “alcohol” solvent refers to a hydrocarbon derivative in which one or more hydrogen atoms have been replaced by an —OH group, known as hydroxyl group. Suitable alcohols include linear, cyclic or branched C₁-C₆ alkyl alcohols and any mixtures thereof. It also includes commercially available alcohols. In another embodiment, the alcohol is methanol, ethanol, isopropanol, 1-propanol and 1-butanol, and mixtures thereof.

As used herein, the term “room temperature” in the context of the present invention refers to a temperature from 15° C. to 30° C., e.g., from 20° C. to 25° C.

As used herein, the term “substantially pure” with reference to a leriglitazone polymorph means that the crystalline material comprises about 10% or less, e.g., about 1% to about 10%, e.g., about 9%, about 8%, about 7%, about 6%, about 5%, about 4%, about 3%, about 2%, or about 1%, by weight of any other crystalline or amorphous form(s), including hydrates or other solvates, of leriglitazone. In another embodiment, the leriglitazone hydrochloride polymorph is substantially pure Form A. In another embodiment, the leriglitazone polymorph is substantially pure Form B. In another embodiment, the leriglitazone polymorph is substantially pure Form C.

As used herein, the term “essentially the same” with reference to XRPD peak positions and/or relative intensities means that peak position and/or intensity variabilities are taken into account when comparing XRPD diffractograms. Likewise, term “essentially the same” with reference to Raman or IR peak positions means that peak position variabilities are taken into account when comparing Raman or IR spectra. For example, XRPD peak positions can show, e.g., inter-apparatus variability, e.g., as much as 0.2° 20, i.e., ±0.2 degrees 2Θ; Raman and IR peak positions can show, e.g., inter-apparatus variability, e.g., as much 4 cm⁻¹, i.e., ±4 cm⁻¹. Relative peak intensities, for example, in a XRPD diffractogram, can also show inter-apparatus variability due to degree of crystallinity, orientation, prepared sample surface, and other factors known to those skilled in the art, and should be taken as qualitative measures only.

As used herein, the term “and/or” is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The articles “a,” “an,” and “the” are used herein to refer to one or to more than one (i.e., to at least one) of the grammatical object of the article. By way of example, “an element” means one element or more than one element.

The term “about,” as used herein, includes the recited number ±10%. Thus, “about 10” means 9 to 11. In another embodiment, the recited number is within 5% of the indicated value. In another embodiment, the recited number is within 1% of the indicated value. In yet another embodiment, the recited number is within 0.5% of the indicated value.

The word “comprising” is used in a manner consistent with its open-ended meaning, that is, to mean that a given product or process can optionally also have additional features or elements beyond those expressly described. It is understood that wherever embodiments are described with the language “comprising,” otherwise analogous embodiments described in terms of “consisting of” and/or “consisting essentially of” are also contemplated and within the scope of this disclosure.

The term “ameliorate” in the context of this present disclosure is understood as meaning any improvement on the situation of the patient treated.

By an “effective” amount or a “therapeutically effective amount” of a drug or pharmacologically active agent is meant a nontoxic but sufficient amount of the drug or agent to provide the desired effect. The amount that is “effective” will vary from patient to patient, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

The term “treatment” or “to treat” and similar terms in the context of this specification means to ameliorate or eliminate the disease or one or more symptoms associated with said disease. “Treatment” also encompasses ameliorating or eliminating the physiological sequelae of the disease.

The term “primary mitochondrial disorder” or “PMD” refers to a mitochondrial disease that can occur due to germline mutations in mitochondrial DNA (mtDNA) and/or nuclear DNA (nDNA) genes encoding the electron transport chain (ETC) proteins and therefore the production of adenosine-triphosphate (ATP), the major cellular energy carrier.

The term “secondary mitochondrial disorder” or “SMD” refers to a mitochondrial disease accompanying many pathologic processes not involving oxidative phosphorylation (OXPHOS), including inherited diseases with germline mutations in non-OXPHOS genes. SMD can also be acquired secondary to adverse environmental effects which can cause oxidative stress.

The term “quantum satis” or “QS” means to add as much of an ingredient to a formulation as is needed to achieve the desired result but not more.

The term “container” means any receptacle and closure therefore suitable for storing, shipping, dispensing, and/or handling a composition of formulation described herein. Non-limiting exemplary containers include vials, ampules, bottles, and syringes.

The term “package insert” means information accompanying a pharmaceutical product that provides a description of how to administer the product, along with the safety and efficacy data required to allow the physician, pharmacist, and subject to make an informed decision regarding use of the product. The package insert generally is regarded as the “label” for a pharmaceutical product.

The term “cellulose derivative” may be cellulose powder, methyl cellulose, microcrystalline cellulose, silicified microcrystalline cellulose, colloidal microcrystalline cellulose (e.g., FMC RC-591), hydroxyethyl cellulose (e.g., Natrosol 250 HX), hydroxypropyl cellulose, hydroxypropyl methyl cellulose (e.g., Benecel K250 PH, Methocel K100 LV, Methocel E50 LV, Methocel E15 LV, and Pharmacoat 615), methylhydroxyethylcellulose, carboxymethylcellulose sodium (e.g., Blanose 9M31F, Blanose 9H4XF, Aqualon 9M31F PH), natural starches, such as maize starch and potato starch; pregelatinized starch, and mixtures thereof. In some embodiments, the ‘cellulose derivative’ is colloidal microcrystalline cellulose (e.g., FMC RC-591), hydroxyethyl cellulose (e.g., Natrosol 250 HX), hydroxypropyl cellulose, hydroxypropyl methyl cellulose; carboxymethylcellulose sodium (e.g., Blanose 9M31F, Blanose 9H4XF), and mixtures thereof.

In some embodiments, the cellulose derivative has a viscosity between 1500 to 4500 mPa*s, e.g., between 1,500 to 3,500 mPa*s, e.g., between 1500 to 3000 mPa*s as measured by Brookfield Viscosity method. In particular, the viscosity may be any value between 1500 and 4500, such as 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500 mPa*s, and any combination thereof as measured by Brookfield Viscosity method.

### IX. PARTICULAR EMBODIMENTS

The disclosure provides the following particular embodiments.

### Embodiment 1

Crystalline leriglitazone hydrate Form C characterized as having:
(i) a x-ray powder diffraction (XRPD) pattern with peaks at 19.1, 21.0, and 23.8 degrees 2Θ using Cu Kα radiation, wherein the 2Θ values are ±0.2 degrees 2Θ; or
(ii) a x-ray powder diffraction pattern with d-spacings at 4.65, 4.23, and 3.73 Å using Cu Kα radiation; or
(iii) a FT-Raman spectrum with peaks at 3056, 2960, 2992, 1735, 1608, 1205, 824, 656, 170 cm⁻¹, wherein the cm⁻¹ values are ±4 cm⁻¹; or
(iv) a melting point with an onset temperature of about 149.45° C. and a peak temperature of about 153.50° C. based on differential scanning calorimetry; or
(v) a combination (i), (ii), (iii), and/or (iv).

### Embodiment 2

The crystalline leriglitazone hydrate Form C of Embodiment 1, characterized as having a XRPD pattern with peaks at 19.1, 21.0, and 23.8 degrees 2Θ using Cu Kα radiation, wherein the 2Θ values are ±0.2 degrees 2Θ.

### Embodiment 3

The crystalline leriglitazone hydrate Form C of any one of Embodiment 1 or 2, further characterized as having a XRPD pattern with peaks at 12.7, 15.4, and 25.9 degrees 2Θ using Cu Kα radiation, wherein the 2Θ values are ±0.2 degrees 2Θ.

### Embodiment 4

The crystalline leriglitazone hydrate Form C of any one of Embodiments 1-3, further characterized as having a XRPD pattern with peaks at 16.9, 21.3, and 21.6 degrees 2Θ using Cu Kα radiation, wherein the 2Θ values are ±0.2 degrees 2Θ.

### Embodiment 5

The crystalline leriglitazone hydrate Form C of any one of Embodiments 1-4, further characterized as having a XRPD pattern with peaks at 7.7, 24.6, and 28.0 degrees 2Θ using Cu Kα radiation, wherein the 2Θ values are ±0.2 degrees 2Θ.

### Embodiment 6

The crystalline leriglitazone hydrate Form C of any one of Embodiments 1-5, further characterized as having a XRPD pattern with peaks at 10.6, 24.9, and 27.3 degrees 2Θ using Cu Kα radiation, wherein the 2Θ values are ±0.2 degrees 2Θ.

### Embodiment 7

The crystalline leriglitazone hydrate Form C of any one of Embodiments 1-6, further characterized as having a XRPD pattern with peaks at 9.2, 19.8, and 29.6 degrees 2Θ using Cu Kα radiation, wherein the 2Θ values are ±0.2 degrees 2Θ.

### Embodiment 8

The crystalline leriglitazone hydrate Form C of Embodiment 1, characterized as having d-spacings at 4.65, 4.23, and 3.73 Å using Cu Kα radiation.

### Embodiment 9

The crystalline leriglitazone hydrate of any one of Embodiments 1 or 8, further characterized as having d-spacings at 6.99, 5.74, and 3.44 Å using Cu Kα radiation.

### Embodiment 10

The crystalline leriglitazone hydrate Form C of any one of Embodiments 1, 8 or 9, further characterized as having d-spacings at 5.24, 4.17, and 4.11 Å using Cu Kα radiation.

### Embodiment 11

The crystalline leriglitazone hydrate Form C of any one of Embodiments 1 or 8-10, further characterized as having d-spacings at 11.41, 3.62, and 3.18 Å using Cu Kα radiation.

### Embodiment 12

The crystalline leriglitazone hydrate Form C of any one of Embodiments 1 or 8-11, further characterized as having d-spacings at 8.32, 3.57, and 3.26 Å using Cu Kα radiation.

### Embodiment 13

The crystalline leriglitazone hydrate Form C of any one of Embodiments 1 or 8-12, further characterized as having d-spacings at 9.60, 4.48, and 3.02 Å using Cu Kα radiation.

### Embodiment 14

The crystalline leriglitazone hydrate Form C of Embodiment 1, characterized as having a FT-Raman spectrum with peaks at 3056, 2960, 2992, 1735, 1608, 1205, 824, 656, 170 cm⁻¹, wherein the cm⁻¹ values are ±4 cm⁻¹;

### Embodiment 15

The crystalline leriglitazone hydrate Form C of any one of Embodiments 1-14, characterized as having about 4.3% loss of mass between 40° C. and 160° C. based on thermal gravimetric analysis.

### Embodiment 16

The crystalline leriglitazone hydrate Form C of any one of Embodiments 1-15, characterized as having a melting point with an onset temperature of about 149.45° C. and a peak temperature of about 153.50° C. based on differential scanning calorimetry.

### Embodiment 17

A pharmaceutical composition comprising a suspension of the crystalline leriglitazone hydrate Form C of any one of Embodiments 1-16 in water and, optionally, one or more pharmaceutically acceptable carriers, diluents or excipients.

### Embodiment 18

Crystalline leriglitazone (unsolvated free base) Form B characterized as having:
(i) a x-ray powder diffraction (XRPD) pattern with peaks at 18.8, 21.1, and 23.8 degrees 2Θ using Cu Kα radiation, wherein the 2Θ values are ±0.2 degrees 2Θ; or
(ii) a x-ray powder diffraction pattern with d-spacings at 4.73, 4.21, and 3.73 Å using Cu Kα radiation; or
(iii) a melting point with an onset temperature of about 149.34° C. and a peak temperature of about 153.56° C. based on differential scanning calorimetry; or
(iv) a combination (i), (ii), and/or (iii).

### Embodiment 19

The crystalline leriglitazone Form B of Embodiment 18, characterized as having a XRPD pattern with peaks at 18.8, 21.1, and 23.8 degrees 2Θ using Cu Kα radiation, wherein the 2Θ values are ±0.2 degrees 2Θ.

### Embodiment 20

The crystalline leriglitazone Form B of any one of Embodiments 18 or 19, further characterized as having a XRPD pattern with peaks at 16.6, 21.8, and 24.7 degrees 2Θ using Cu Kα radiation, wherein the 2Θ values are ±0.2 degrees 2Θ.

### Embodiment 21

The crystalline leriglitazone Form B of any one of Embodiments 18-20, further characterized as having a XRPD pattern with peaks at 15.2, 25.4, and 28.0 degrees 2Θ using Cu Kα radiation, wherein the 2Θ values are ±0.2 degrees 2Θ.

### Embodiment 22

The crystalline leriglitazone Form B of any one of Embodiments 18-21, further characterized as having a XRPD pattern with peaks at 9.0, 19.8, and 30.8 degrees 2Θ using Cu Kα radiation, wherein the 2Θ values are ±0.2 degrees 2Θ.

### Embodiment 23

The crystalline leriglitazone Form B of any one of Embodiments 21-22, further characterized as having a XRPD pattern with peaks at 10.5, 20.1, and 26.9 degrees 2Θ using Cu Kα radiation, wherein the 2Θ values are ±0.2 degrees 2Θ.

### Embodiment 24

The crystalline leriglitazone Form B of any one of Embodiments 21-23, further characterized as having a XRPD pattern with peaks at 12.4, 23.5, and 29.1 degrees 2Θ using Cu Kα radiation, wherein the 2Θ values are ±0.2 degrees 2Θ.

### Embodiment 25

The crystalline leriglitazone Form B of Embodiment 18, characterized as having d-spacings at 4.73, 4.21, and 3.73 Å using Cu Kα radiation.

### Embodiment 26

The crystalline leriglitazone Form B of any one of Embodiments 18 or 25, further characterized as having d-spacings at 5.33, 4.07, and 3.60 Å using Cu Kα radiation.

### Embodiment 27

The crystalline leriglitazone Form B of any one of Embodiments 18, 25 or 26, further characterized as having d-spacings at 5.84, 3.50, and 3.18 Å using Cu Kα radiation.

### Embodiment 28

The crystalline leriglitazone Form B of any one of Embodiments 18 or 25-27, further characterized as having d-spacings at 9.78, 4.48, and 2.90 Å using Cu Kα radiation.

### Embodiment 29

The crystalline leriglitazone Form B of any one of Embodiments 18 or 25-28, further characterized as having d-spacings at 8.43, 4.41, and 3.31 Å using Cu Kα radiation.

### Embodiment 30

The crystalline leriglitazone Form B of any one of Embodiments 18 or 25-29, further characterized as having d-spacings at 7.11, 3.79, and 3.06 Å using Cu Kα radiation.

### Embodiment 31

The crystalline leriglitazone Form B of any one of Embodiments 18-30, characterized as having a melting point with an onset temperature of about 149.34° C. and a peak temperature of about 153.56° C. based on differential scanning calorimetry.

### Embodiment 32

The crystalline leriglitazone Form B of any one of Embodiments 18-31, characterized as having about 0.9% loss of mass between 40° C. and 100° C. based on thermal gravimetric analysis.

### Embodiment 33

A pharmaceutical composition comprising a suspension of the crystalline leriglitazone Form B of any one of Embodiments 18-32 in water, and one or more pharmaceutically acceptable carriers, diluents or excipients.

### Embodiment 34

A pharmaceutical formulation comprising:
(a) leriglitazone, a pharmaceutically acceptable salt thereof, or a hydrate thereof, in an amount from 0.01% w/v to 5% w/v;
(b) one or more cellulose derivatives in an amount from 0.01% w/v to 2% w/v, wherein the one or more cellulose derivatives have a viscosity between 1500 and 4500 mPa*s, preferably between 1500 to 3500 mPa*s, more preferably between 1500 and 3000 mPa*s as measured by Brookfield viscosity method; and
(c) water QS to 100%.

### Embodiment 35

The pharmaceutical formulation according to Embodiment 34, wherein the leriglitazone, pharmaceutically acceptable salt thereof, or a hydrate thereof, is leriglitazone Form B, characterized as having (i) a XRPD pattern with peaks at 18.8, 21.1, and 23.8 degrees 2Θ using Cu Kα radiation, wherein the 2Θ values are ±0.2 degrees 2θ; or (ii) a mass loss of about 0.9% between 40° C. to about 100° C. based on thermal gravimetric analysis.

### Embodiment 36

The pharmaceutical formulation according to Embodiment 34, wherein the leriglitazone, pharmaceutically acceptable salt thereof, or a hydrate thereof, is leriglitazone hydrated Form C, characterized as having (i) a XRPD pattern with peaks at 19.1, 21.0, and 23.8 degrees 2θ using Cu Kα radiation, wherein the 2Θ values are ±0.2 degrees 2θ; or (ii) a mass loss of about 4.3% between 40° C. and 160° C. based on thermal gravimetric analysis.

### Embodiment 37

The pharmaceutical formulation according to Embodiment 34, wherein the leriglitazone, pharmaceutically acceptable salt thereof, or a hydrate thereof, is present in an amount from 1% w/v to 2% w/v.

### Embodiment 38

The pharmaceutical formulation according to Embodiment 35, wherein the leriglitazone Form B is present in an amount from 1% w/v to 2% w/v.

### Embodiment 39

The pharmaceutical formulation according to Embodiment 36, wherein the leriglitazone hydrate Form C is present in an amount from 1% w/v to 2% w/v.

### Embodiment 40

The pharmaceutical formulation according to any one of Embodiments 34-39, wherein the one or more cellulose derivatives comprise microcrystalline cellulose, carboxymethylcellulose sodium, or mixtures thereof.

### Embodiment 41

The pharmaceutical formulation according to any one of Embodiments 34-40 further comprising one or more pH regulators.

### Embodiment 42

The pharmaceutical formulation according to Embodiment 41, wherein the one or more pH regulators comprise sodium citrate, citric acid monohydrate, or mixtures thereof.

### Embodiment 43

The pharmaceutical formulation according to any one of Embodiments 34-42 further comprising one or more preservatives in an amount from 0.001% w/v to 1.0% w/v.

### Embodiment 44

The pharmaceutical formulation according to Embodiment 43, wherein the one or more preservatives comprises sodium benzoate.

### Embodiment 45

The pharmaceutical formulation according to any one of Embodiments 34-44 further comprising one or more flavouring agents in an amount of 0.001% w/v to 3% w/v.

### Embodiment 46

The pharmaceutical formulation according to Embodiment 45, wherein the one or more flavouring agents comprise strawberry flavor.

### Embodiment 47

The pharmaceutical formulation according to any one of Embodiments 34-46 further comprising one or more sweetening agents in an amount of 0.01% w/v to 15% w/v.

### Embodiment 48

The pharmaceutical formulation according to Embodiment 47, wherein the sweetening agent is sorbitol, saccharin sodium, or mixtures thereof.

### Embodiment 49

The pharmaceutical formulation according to any one of Embodiments 34-48, wherein the pH of the formulation is of 3.5 to 4.5.

### Embodiment 50

The pharmaceutical formulation according to Embodiment 49, wherein the pH of the formulation is about 4.

### Embodiment 51

The pharmaceutical formulation according to any one of Embodiments 34-50, wherein the viscosity of the formulation is higher than 50 mPa*s and less than 5000 mPa*s, preferably less than 4,500 mPa*s.

### Embodiment 52

The pharmaceutical formulation of Embodiment 34 comprising:
(a) leriglitazone, a pharmaceutically acceptable salt, or a hydrate thereof, preferably hydrate Form C; in an amount of 1.0% to 2.0% w/v; (b) about 1% w/v of microcrystalline cellulose; (c) about 0.5% w/v of carboxymethyl cellulose sodium; (d) water QS to 100%; and, optionally: (e) about 8% w/v sorbitol powder; (f) about 0.05% w/v saccharin sodium; (g) about 0.1% w/v sodium benzoate; (h) about 0.5% w/v sodium citrate; (i) about 0.1% w/v citric acid monohydrate; (j) about 0.015% w/v strawberry flavour; and (k) about 1% w/v 0.2 M aqueous citric acid monohydrate solution.

### Embodiment 53

A pharmaceutical formulation prepared by admixing: (a) about 1.5% w/v leriglitazone hydrochloride, e.g., Form A; (b) about 8% w/v sorbitol powder; (c) about 1% w/v microcrystalline cellulose; (d) about 0.5% w/v carboxymethyl cellulose sodium; (e) about 0.05% w/v saccharin sodium; (f) about 0.1% w/v sodium benzoate; (g) about 0.5% w/v sodium citrate; (h) about 0.1% w/v citric acid monohydrate; (i) about 0.015% w/v strawberry flavour; (j) about 1% w/v 0.2 M aqueous citric acid monohydrate solution; and (k) water QS to 100%.

### Embodiment 54

The crystalline leriglitazone hydrate Form C of any one of Embodiments 1-16 or the pharmaceutical composition of Embodiment 17, or the crystalline leriglitazone Form B of any one of Embodiments 18-32 or the pharmaceutical composition of Embodiment 33, or the pharmaceutical formulation of any one of Embodiments 34-53 for use in treating a disease or disorder in a patient in need thereof, wherein the disease or disorder is a central nervous system disease or disorder, mitochondrial disease, a liver disease or disorder, chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, an inflammatory respiratory disease, or a lung disease or disorder.

### Embodiment 55

The crystalline leriglitazone form or the pharmaceutical composition or formulation of Embodiment 54, wherein the disease or disorder is a central nervous system disease or disorder.

### Embodiment 56

The crystalline leriglitazone form or the pharmaceutical composition or formulation of Embodiment 55, wherein the central nervous system disease or disorder is selected from the group consisting of neurodegenerative disease, cerebrovascular disease, seizure, epilepsy, viral disease, neuroinflammatory disease, brain tumor, organic acidemias, fatty acid disorder, and genetic mitochondrial disorder.

### Embodiment 57

The crystalline leriglitazone form or the pharmaceutical composition or formulation of Embodiment 56, wherein the central nervous system disease or disorder is a neurodegenerative disease.

### Embodiment 58

The crystalline leriglitazone form or the pharmaceutical composition or formulation of Embodiment 57, wherein the neurodegenerative disease is selected from the group consisting of Alzheimer's disease, Huntington's chorea, Parkinson's disease, multiple sclerosis, neuromyelitis optica, leukodystrophy, amyotrophic lateral sclerosis (ALS), degenerative ataxia, multiple system atrophy, neurodegeneration and brain iron accumulation disorders (NBIA), neuromyopathy, and a motor neuron disease.

### Embodiment 59

The crystalline leriglitazone form or the pharmaceutical composition or formulation of Embodiment 58, wherein the leukodystrophy is X-linked adrenoleukodystrophy, adrenomyeloneuropathy, cerebral adrenoleukodystrophy, or metachromatic leukodystrophy.

### Embodiment 60

The crystalline leriglitazone form or the pharmaceutical composition or formulation of Embodiment 59, wherein the leukodystrophy is cerebral adrenoleukodystrophy.

### Embodiment 61

The crystalline leriglitazone form or the pharmaceutical composition or formulation of Embodiment 58, wherein the degenerative ataxia is Friedreich's ataxia.

### Embodiment 62

The crystalline leriglitazone form or the pharmaceutical composition or formulation of Embodiment 58, wherein the motor neuron disease is selected from the group consisting of progressive bulbar palsy, pseudobulbar palsy, primary lateral sclerosis (PLS), progressive muscular atrophy, spinal muscular atrophy (SMA), post-polio syndrome (PPS)-Marie-Tooth disease, Guillan-Barré syndrome, and adrenomyeloneuropathy (AMN).

### Embodiment 63

The crystalline leriglitazone form or the pharmaceutical composition or formulation of Embodiment 56, wherein the cerebrovascular disease is selected from the group consisting of global or local ischemia, intracerebral haemorrhage, stroke, and vascular dementia.

### Embodiment 64

The crystalline leriglitazone form or the pharmaceutical composition or formulation of Embodiment 56, the viral disease is selected from the group consisting of meningitis, encephalitis, rabies, measles, mumps, poliomyelitis, herpes simplex, and varicella zoster.

### Embodiment 65

The crystalline leriglitazone form or the pharmaceutical composition or formulation of Embodiment 55, wherein the central nervous system disease or disorder is a rare metabolic disease selected from the group consisting of organic acidemias, fatty acid disorders and genetic mitochondrial disorders.

### Embodiment 66

The crystalline leriglitazone form or the pharmaceutical composition or formulation of Embodiment 54, wherein the disease or disorder is a mitochondrial disease.

### Embodiment 67

The crystalline leriglitazone form or the pharmaceutical composition or formulation of Embodiment 66, wherein the mitochondrial disease is a primary mitochondrial disorder selected from the group consisting of Rett syndrome, Alper's disease; Leber's hereditary optic neuropathy (LHON); Kearns-Sayre syndrome (KSS); Leigh's syndrome; Leigh-like syndrome; maternally inherited Leigh syndrome (MILS); mitochondrial depletion syndrome (MDS); mitochondrial DNA depletion syndrome (MDDS); mitochondrial encephalomyopathy; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; chronic progressive external opthalmoplegia (CPEO); dominant optic atrophy (DOA); autosomal dominant optic atrophy (ADOA); mitochondrial myopathy; cardiomyopathy; mitochondrial encephalopathy; myoclonic epilepsy; maternally inherited diabetes and deafness (MIDD); ataxia neuropathy spectrum; 3-methylglutaconic aciduria; sensoneural deafness; neuroradiological findings of Leigh-like syndrome (MEGDEL); SURF1 (COX definient Leigh syndrome due to complex IV surfeit protein deficiency); oxidative phosphorylation disorders; Berth syndrome; lethal infantile cardiomyopathy (LIC); pyruvate carboxylase deficiency; pyruvate dehydrogenase deficiency; POLG mutation; isolated or combined OXPHOS deficiencies with so far unsolved genetic defect including disturbed pyruvate oxidation and ATP plus PCr production rates; POLG2 mutation; carnitine-acyl-cartinine deficiency; carnitine deficiency; creatinine deficiency syndromes; Co-Enzyme Q10 deficiency; Complex I deficiency; Complex II deficiency; Complex III deficiency; Complex IV deficiency; Complex V deficiency; lactic acidosis; leukoencephalopathy with brain stem and spinal cord involvement and lactate elevation (LBSL); Luft disease; carnitine palmitoyltransferase (CPT I or CPT II) deficiency; short-chain acyl-CoA dehydrogenase deficiency (SCAD); short-chain 3-hydroxyacetyl-CoA dehydrogenase deficiency (SCHAD); medium-chain acyl-CoA dehydrogenase deficiency (MCAD); multiple acyl-CoA dehydrogenase deficiency (MADD); long-chain acyl-CoA dehydrogenase deficiency (LCAD); very long-chain acyl-CoA dehydrogenase deficiency (VLCAD); trifunctional protein (TFP) deficiency; and glutaric aciduria Type II.

### Embodiment 68

The crystalline leriglitazone form or the pharmaceutical composition or formulation of Embodiment 67, wherein the mitochondrial disease is selected from the group consisting of Rett syndrome; dominant optic atrophy (DOA); autosomal dominant optic atrophy (ADOA); Complex I deficiency; Leber hereditary optic neuropathy (LHON); Kearns-Sayre syndrome (KSS); Leigh's syndrome; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; and chronic progressive external opthalmoplegia (CPEO).

### Embodiment 69

The crystalline leriglitazone form or the pharmaceutical composition or formulation of Embodiment 66, wherein the mitochondrial disease is a secondary mitochondrial disorder selected from the group consisting of Duchenne muscular dystrophy (DMD); Becker muscular dystrophy (BMD); myotonic dystrophy (BMD); congenital myopathies; glycogen storage disorders; spinal-bulbar muscular atrophy (SBMA); argininosuccinic aciduria; autism spectrum disorder (ASD); autoimmune diseases of the skin (such as pemphigus vulgaris and lupus); methylmalonic and propionic acidurias; disorders or purine and/or pyrimidine synthesis; facioscapulohumeral muscular dystrophy (FSHD); congenital muscular dystrophies; collagen VI muscular dystrophies (e.g., Ullrich congenital muscular dystrophy, Bethlem myopathy, oculopharyngeal distal and Emery-Dreifuss); DiGeorge syndrome; and neuromuscular disorders (such as limb-girdle muscular dystrophy, inflammatory myopathies, Charcot Marie Tooth (CMT) neuropathy, and drug-induced peripheral neuropathies).

### Embodiment 70

The crystalline leriglitazone form or the pharmaceutical composition of Embodiment 54, wherein the lung disease or disorder is an inflammatory lung condition or disease caused by a viral infection, acute inflammation of the lung, or interstitial lung disease.

### Embodiment 71

The crystalline leriglitazone form or the pharmaceutical composition or formulation of Embodiment 70 for treating an inflammatory lung condition or disease caused by a viral infection in a patient in need thereof.

### Embodiment 72

The crystalline leriglitazone form or the pharmaceutical composition or formulation of Embodiment 71, wherein the viral infection is a human coronavirus infection, an influenza virus infection, or a HIV virus infection.

### Embodiment 73

The crystalline leriglitazone form or the pharmaceutical composition or formulation of Embodiment 72, wherein the human coronavirus is HCoV 229E, HCOV OC43, HCOV NL63, HCOV HKU1, SARS COV, MERS COV, or SARS COV-2, or a mutated strain thereof.

### Embodiment 74

The crystalline leriglitazone form or the pharmaceutical composition or formulation of Embodiment 73, wherein the human coronavirus is SARS COV-2.

### Embodiment 75

The crystalline leriglitazone form or the pharmaceutical composition or formulation of Embodiment 74, wherein the human coronavirus is a mutated strain of SARS COV 2.

### Embodiment 76

The crystalline leriglitazone form or the pharmaceutical composition or formulation of any one of Embodiments 71-75, wherein the inflammatory lung condition or disease caused by the viral infection is hypercytokinaemia, haemophagocytic lymphohistiocytosis, pneumonia, acute respiratory distress syndrome, or systemic inflammatory response syndrome.

### Embodiment 77

The crystalline leriglitazone form or the pharmaceutical composition or formulation of Embodiment 76, wherein the inflammatory lung condition or disease caused by the viral infection is acute respiratory distress syndrome.

### Embodiment 78

The crystalline leriglitazone form or the pharmaceutical composition or formulation of Embodiment 70 for treating acute inflammation of the lung in a patient in need thereof.

### Embodiment 79

The crystalline leriglitazone form or the pharmaceutical composition or formulation of Embodiment 78, wherein the acute inflammation of the lung is caused by a bacterial infection.

### Embodiment 80

The crystalline leriglitazone form or the pharmaceutical composition or formulation of Embodiment 78, wherein the acute inflammation of the lung is pneumonia or acute respiratory distress syndrome.

### Embodiment 81

The crystalline leriglitazone form or the pharmaceutical composition or formulation of Embodiment 70 for treating interstitial lung disease in a patient in need thereof.

### Embodiment 82

The crystalline leriglitazone form or the pharmaceutical composition or formulation of Embodiment 70, wherein the interstitial lung disease is idiopathic pulmonary fibrosis.

### Embodiment 83

The crystalline leriglitazone form or the pharmaceutical composition or formulation of Embodiment 54, wherein the liver disease or disorder is nonalcoholic steatohepatitis (NASH) or nonalcoholic fatty liver disease (NAFLD).

### Embodiment 84

A method of making crystalline leriglitazone hydrate Form C of Embodiment 1, the method comprising the following steps:
(a) providing leriglitazone hydrochloride salt, preferably Form A, in water or in mixtures of water with an alcohol solvent to provide a solution or a suspension,
(b) optionally, heating the mixture of step (a) at a suitable temperature, preferably from 40° C. to 100° C., more preferably from 50° C. to 85° C.,
(c) optionally, cooling the solution or the suspension resulting from step (b) to room temperature, and
(d) isolating the solid obtained in step (c) providing Form C of leriglitazone.

### Embodiment 85

A method of making crystalline leriglitazone Form B of Embodiment 18, the method comprising the following steps:
(a) providing leriglitazone hydrochloride salt, e.g., Form A, in water or in mixtures of water with an alcohol solvent to provide a solution or a suspension,
(b) optionally, heating the mixture of step (a) at a suitable temperature, preferably from 40° C. to 100° C., more preferably from 50° C. to 85° C.,
(c) optionally, cooling the solution or the suspension resulting from step (b) to room temperature, and
(d) isolating and drying the solid obtained in step (c) providing Form B of leriglitazone.

### Embodiment 86

A kit comprising the pharmaceutical composition of any one of Embodiments 34-53 in a container and (ii) a label with instructions how to use the kit.

### Embodiment 87

The kit of Embodiment 86, wherein the label is approved by the United States Food and Drug Administration (FDA), the European Medicines Agency (EMA), the China Food and Drug Administration (CFDA), or the Japanese Ministry of Health Labor and Welfare (MHLW).

### Embodiment 88

The kit of Embodiments 86 or 87, further comprising a graduated oral syringe of 5 mL, 10 mL, 12 mL, or 15 mL

### Embodiment 89

A method of treating a disease or disorder in a patient in need thereof, the method comprising administering a therapeutically effective amount of the leriglitazone hydrate Form C of any one of Embodiments 1-16 or the pharmaceutical composition of Embodiment 17, or the crystalline leriglitazone Form B of any one of Embodiments 18-32 or the pharmaceutical composition of Embodiment 33, or the pharmaceutical formulation of any one of Embodiments 34-53 to the patient, wherein the disease or disorder is a central nervous system disease or disorder, mitochondrial disease, a liver disease or disorder, chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, an inflammatory respiratory disease, or a lung disease or disorder.

### Embodiment 90

The method of Embodiment 89, wherein the disease or disorder is a central nervous system disease or disorder.

### Embodiment 91

The method of Embodiment 90, wherein the central nervous system disease or disorder is selected from the group consisting of neurodegenerative disease, cerebrovascular disease, seizure, epilepsy, viral disease, neuroinflammatory disease, brain tumor, organic acidemias, fatty acid disorder, and genetic mitochondrial disorder.

### Embodiment 92

The method of Embodiment 91, wherein the central nervous system disease or disorder is a neurodegenerative disease.

### Embodiment 93

The method of Embodiment 92, wherein the neurodegenerative disease is selected from the group consisting of Alzheimer's disease, Huntington's chorea, Parkinson's disease, multiple sclerosis, neuromyelitis optica, leukodystrophy, ALS, degenerative ataxia, multiple system atrophy, NBIA (neurodegeneration and brain iron accumulation disorders), neuromyopathy, and a motor neuron disease.

### Embodiment 94

The method of Embodiment 93, wherein the leukodystrophy is X-linked adrenoleukodystrophy, adrenomyeloneuropathy, cerebral adrenoleukodystrophy, or metachromatic leukodystrophy.

### Embodiment 95

The method of Embodiment 94, wherein the leukodystrophy is cerebral adrenoleukodystrophy.

### Embodiment 96

The method of Embodiment 93, wherein the degenerative ataxia is Friedreich's ataxia.

### Embodiment 97

The method of Embodiment 93, wherein the motor neuron disease is selected from the group consisting of progressive bulbar palsy, pseudobulbar palsy, primary lateral sclerosis (PLS), progressive muscular atrophy, spinal muscular atrophy (SMA), post-polio syndrome (PPS)-Marie-Tooth disease, Guillan-Barré syndrome, and adrenomyeloneuropathy (AMN)

### Embodiment 98

The method of Embodiment 91, wherein the central nervous system disorder is a cerebrovascular disease selected from the group consisting of global or local ischemia, intracerebral haemorrhage, stroke, and vascular dementia.

### Embodiment 99

The method of Embodiment 91, the central nervous system disorder is a viral disease selected from the group consisting of meningitis, encephalitis, rabies, measles, mumps, poliomyelitis, herpes simplex, and varicella zoster.

### Embodiment 100

The method of Embodiment 91, wherein the central nervous system disease or disorder is a rare metabolic disease selected from the group consisting of organic acidemias, fatty acid disorders and genetic mitochondrial disorders.

### Embodiment 101

The method of Embodiment 89, wherein the disease or disorder is a mitochondrial disease.

### Embodiment 102

The method of Embodiment 101, wherein the mitochondrial disease is a primary mitochondrial disorder selected from the group consisting of Rett syndrome, Alper's disease; Leber's hereditary optic neuropathy (LHON); Kearns-Sayre syndrome (KSS); Leigh's syndrome; Leigh-like syndrome; maternally inherited Leigh syndrome (MILS); mitochondrial depletion syndrome (MDS); mitochondrial DNA depletion syndrome (MDDS); mitochondrial encephalomyopathy; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; chronic progressive external opthalmoplegia (CPEO); dominant optic atrophy (DOA); autosomal dominant optic atrophy (ADOA); mitochondrial myopathy; cardiomyopathy; mitochondrial encephalopathy; myoclonic epilepsy; maternally inherited diabetes and deafness (MIDD); ataxia neuropathy spectrum; 3-methylglutaconic aciduria; sensoneural deafness; neuroradiological findings of Leigh-like syndrome (MEGDEL); SURF1 (COX definient Leigh syndrome due to complex IV surfeit protein deficiency); oxidative phosphorylation disorders; Berth syndrome; lethal infantile cardiomyopathy (LIC); pyruvate carboxylase deficiency; pyruvate dehydrogenase deficiency; POLG mutation; isolated or combined OXPHOS deficiencies with so far unsolved genetic defect including disturbed pyruvate oxidation and ATP plus PCr production rates; POLG2 mutation; carnitine-acyl-cartinine deficiency; carnitine deficiency; creatinine deficiency syndromes; Co-Enzyme Q10 deficiency;

Complex I deficiency; Complex II deficiency; Complex III deficiency; Complex IV deficiency; Complex V deficiency; lactic acidosis; leukoencephalopathy with brain stem and spinal cord involvement and lactate elevation (LBSL); Luft disease; carnitine palmitoyltransferase (CPT I or CPT II) deficiency; short-chain acyl-CoA dehydrogenase deficiency (SCAD); short-chain 3-hydroxyacetyl-CoA dehydrogenase deficiency (SCHAD); medium-chain acyl-CoA dehydrogenase deficiency (MCAD); multiple acyl-CoA dehydrogenase deficiency (MADD); long-chain acyl-CoA dehydrogenase deficiency (LCAD); very long-chain acyl-CoA dehydrogenase deficiency (VLCAD); trifunctional protein (TFP) deficiency; and glutaric aciduria Type II.

### Embodiment 103

The method of Embodiment 102, wherein the mitochondrial disease is selected from the group consisting of Rett syndrome; dominant optic atrophy (DOA); autosomal dominant optic atrophy (ADOA); Complex I deficiency; Leber hereditary optic neuropathy (LHON); Kearns-Sayre syndrome (KSS); Leigh's syndrome; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; and chronic progressive external opthalmoplegia (CPEO).

### Embodiment 104

The method of Embodiment 101, wherein the mitochondrial disease is a secondary mitochondrial disorder selected from the group consisting of Duchenne muscular dystrophy (DMD); Becker muscular dystrophy (BMD); myotonic dystrophy (BMD); congenital myopathies; glycogen storage disorders; spinal-bulbar muscular atrophy (SBMA); argininosuccinic aciduria; autism spectrum disorder (ASD); autoimmune diseases of the skin (such as pemphigus vulgaris and lupus); methylmalonic and propionic acidurias; disorders or purine and/or pyrimidine synthesis; facioscapulohumeral muscular dystrophy (FSHD); congenital muscular dystrophies; collagen VI muscular dystrophies (e.g., Ullrich congenital muscular dystrophy, Bethlem myopathy, oculopharyngeal distal and Emery-Dreifuss); DiGeorge syndrome; and neuromuscular disorders (such as limb-girdle muscular dystrophy, inflammatory myopathies, Charcot Marie Tooth (CMT) neuropathy, and drug-induced peripheral neuropathies).

### Embodiment 105

The method of Embodiment 89, wherein the lung disease or disorder is an inflammatory lung condition or disease caused by a viral infection, acute inflammation of the lung, or interstitial lung disease.

### Embodiment 106

The method of Embodiment 105 for treating an inflammatory lung condition or disease caused by a viral infection in a patient in need thereof.

### Embodiment 107

The method of Embodiment 106, wherein the viral infection is a human coronavirus infection, an influenza virus infection, or a HIV virus infection.

### Embodiment 108

The method of Embodiment 107, wherein the human coronavirus is HCOV 229E, HCOV OC43, HCOV NL63, HCOV HKU1, SARS COV, MERS CoV, or SARS COV-2, or a mutated strain thereof.

### Embodiment 109

The method of Embodiment 108, wherein the human coronavirus is SARS COV-2.

### Embodiment 110

The method of Embodiment 109, wherein the human coronavirus is a mutated strain of SARS COV-2.

### Embodiment 111

The method of any one of Embodiments 106-110, wherein the inflammatory lung condition or disease caused by the viral infection is hypercytokinaemia, haemophagocytic lymphohistiocytosis, pneumonia, acute respiratory distress syndrome, or systemic inflammatory response syndrome.

### Embodiment 112

The method of Embodiment 111, wherein the inflammatory lung condition or disease caused by the viral infection is acute respiratory distress syndrome.

### Embodiment 113

The method of Embodiment 105 for treating acute inflammation of the lung in a patient in need thereof.

### Embodiment 114

The method of Embodiment 113, wherein the acute inflammation of the lung is caused by a bacterial infection.

### Embodiment 115

The method of Embodiment 114, wherein the acute inflammation of the lung is pneumonia or acute respiratory distress syndrome.

### Embodiment 116

The method of Embodiment 105 for treating interstitial lung disease in a patient in need thereof.

### Embodiment 117

The method of Embodiment 116, wherein the interstitial lung disease is idiopathic pulmonary fibrosis.

### Embodiment 118

The method of Embodiment 89, wherein the liver disease or disorder is nonalcoholic steatohepatitis (NASH) or nonalcoholic fatty liver disease (NAFLD).

### Embodiment 119

A method of making crystalline leriglitazone Form B, the method comprising the following steps: (a) providing leriglitazone hydrochloride salt, in water or in mixtures of water with an alcohol solvent to provide a solution or a suspension, (b) optionally, heating the mixture of step (a) at a suitable temperature, preferably from 40° C. to 100° C., more preferably from 50° C. to 85° C., (c) optionally, cooling the solution or the suspension resulting from step (b) to room temperature, and (d) isolating and drying the solid obtained in step (c) providing leriglitazone Form B.

### Embodiment 120

A method of making crystalline leriglitazone hydrate Form C, the method comprising the following steps: (a) providing leriglitazone hydrochloride salt, in water or in mixtures of water with an alcohol solvent to provide a solution or a suspension, (b) optionally, heating the mixture of step (a) at a suitable temperature, preferably from 40° C. to 100° C., more preferably from 50° C. to 85° C., (c) optionally, cooling the solution or the suspension resulting from step (b) to room temperature, and (d) isolating the solid obtained in step (c) providing leriglitazone hydrate Form C.

### Embodiment 121

Use of the crystalline leriglitazone hydrate Form C of any one of Embodiments 1-16, or the pharmaceutical composition of Embodiment 17, or the crystalline leriglitazone Form B of any one of Embodiments 18-32, or the pharmaceutical composition of Embodiment 33, or the pharmaceutical formulation of any one of Embodiments 34-53 in the manufacture of a medicament for treating a disease or disorder in a patient in need thereof, wherein the disease or disorder is a central nervous system disease or disorder, mitochondrial disease, a liver disease or disorder, chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, an inflammatory respiratory disease, or a lung disease or disorder.

### Embodiment 122

The use of Embodiment 121, wherein the disease or disorder is a central nervous system disease or disorder.

### Embodiment 123

The use of Embodiment 122, wherein the central nervous system disease or disorder is selected from the group consisting of neurodegenerative disease, cerebrovascular disease, seizure, epilepsy, viral disease, neuroinflammatory disease, brain tumor, organic acidemias, fatty acid disorder, and genetic mitochondrial disorder.

### Embodiment 124

The use of Embodiment 123, wherein the central nervous system disease or disorder is a neurodegenerative disease.

### Embodiment 125

The use of Embodiment 124, wherein the neurodegenerative disease is selected from the group consisting of Alzheimer's disease, Huntington's chorea, Parkinson's disease, multiple sclerosis, neuromyelitis optica, leukodystrophy, amyotrophic lateral sclerosis (ALS), degenerative ataxia, multiple system atrophy, neurodegeneration and brain iron accumulation disorders (NBIA), neuromyopathy, and a motor neuron disease.

### Embodiment 126

The use of Embodiment 125, wherein the leukodystrophy is X-linked adrenoleukodystrophy, adrenomyeloneuropathy, cerebral adrenoleukodystrophy, or metachromatic leukodystrophy.

### Embodiment 127

The use of Embodiment 126, wherein the leukodystrophy is cerebral adrenoleukodystrophy.

### Embodiment 128

The use of Embodiment 125, wherein the degenerative ataxia is Friedreich's ataxia.

### Embodiment 129

The use of Embodiment 58125 wherein the motor neuron disease is selected from the group consisting of progressive bulbar palsy, pseudobulbar palsy, primary lateral sclerosis (PLS), progressive muscular atrophy, spinal muscular atrophy (SMA), post-polio syndrome (PPS)-Marie-Tooth disease, Guillan-Barré syndrome, and adrenomyeloneuropathy (AMN).

### Embodiment 130

The use of Embodiment 123, wherein the cerebrovascular disease is selected from the group consisting of global or local ischemia, intracerebral haemorrhage, stroke, and vascular dementia.

### Embodiment 131

The use of Embodiment 123, the viral disease is selected from the group consisting of meningitis, encephalitis, rabies, measles, mumps, poliomyelitis, herpes simplex, and varicella zoster.

### Embodiment 132

The use of Embodiment 122, wherein the central nervous system disease or disorder is a rare metabolic disease selected from the group consisting of organic acidemias, fatty acid disorders and genetic mitochondrial disorders.

### Embodiment 133

The use of Embodiment 121, wherein the disease or disorder is a mitochondrial disease.

### Embodiment 134

The use of Embodiment 133, wherein the mitochondrial disease is a primary mitochondrial disorder selected from the group consisting of Rett syndrome, Alper's disease; Leber's hereditary optic neuropathy (LHON); Kearns-Sayre syndrome (KSS); Leigh's syndrome; Leigh-like syndrome; maternally inherited Leigh syndrome (MILS); mitochondrial depletion syndrome (MDS); mitochondrial DNA depletion syndrome (MDDS); mitochondrial encephalomyopathy; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; chronic progressive external opthalmoplegia (CPEO); dominant optic atrophy (DOA); autosomal dominant optic atrophy (ADOA); mitochondrial myopathy; cardiomyopathy; mitochondrial encephalopathy; myoclonic epilepsy; maternally inherited diabetes and deafness (MIDD); ataxia neuropathy spectrum; 3-methylglutaconic aciduria; sensoneural deafness; neuroradiological findings of Leigh-like syndrome (MEGDEL); SURF1 (COX definient Leigh syndrome due to complex IV surfeit protein deficiency); oxidative phosphorylation disorders; Berth syndrome; lethal infantile cardiomyopathy (LIC); pyruvate carboxylase deficiency; pyruvate dehydrogenase deficiency; POLG mutation; isolated or combined OXPHOS deficiencies with so far unsolved genetic defect including disturbed pyruvate oxidation and ATP plus PCr production rates; POLG2 mutation; carnitine-acyl-cartinine deficiency; carnitine deficiency; creatinine deficiency syndromes; Co-Enzyme Q10 deficiency; Complex I deficiency; Complex II deficiency; Complex III deficiency; Complex IV deficiency; Complex V deficiency; lactic acidosis; leukoencephalopathy with brain stem and spinal cord involvement and lactate elevation (LBSL); Luft disease; carnitine palmitoyltransferase (CPT I or CPT II) deficiency; short-chain acyl-CoA dehydrogenase deficiency (SCAD); short-chain 3-hydroxyacetyl-CoA dehydrogenase deficiency (SCHAD); medium-chain acyl-CoA dehydrogenase deficiency (MCAD); multiple acyl-CoA dehydrogenase deficiency (MADD); long-chain acyl-CoA dehydrogenase deficiency (LCAD); very long-chain acyl-CoA dehydrogenase deficiency (VLCAD); trifunctional protein (TFP) deficiency; and glutaric aciduria Type II.

### Embodiment 135

The use of Embodiment 134, wherein the mitochondrial disease is selected from the group consisting of Rett syndrome; dominant optic atrophy (DOA); autosomal dominant optic atrophy (ADOA); Complex I deficiency; Leber hereditary optic neuropathy (LHON); Kearns-Sayre syndrome (KSS); Leigh's syndrome; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; and chronic progressive external opthalmoplegia (CPEO).

### Embodiment 136

The use of Embodiment 133, wherein the mitochondrial disease is a secondary mitochondrial disorder selected from the group consisting of Duchenne muscular dystrophy (DMD); Becker muscular dystrophy (BMD); myotonic dystrophy (BMD); congenital myopathies; glycogen storage disorders; spinal-bulbar muscular atrophy (SBMA); argininosuccinic aciduria; autism spectrum disorder (ASD); autoimmune diseases of the skin (such as pemphigus vulgaris and lupus); methylmalonic and propionic acidurias; disorders or purine and/or pyrimidine synthesis; facioscapulohumeral muscular dystrophy (FSHD); congenital muscular dystrophies; collagen VI muscular dystrophies (e.g., Ullrich congenital muscular dystrophy, Bethlem myopathy, oculopharyngeal distal and Emery-Dreifuss); DiGeorge syndrome; and neuromuscular disorders (such as limb-girdle muscular dystrophy, inflammatory myopathies, Charcot Marie Tooth (CMT) neuropathy, and drug-induced peripheral neuropathies).

### Embodiment 137

The use of Embodiment 121, wherein the lung disease or disorder is an inflammatory lung condition or disease caused by a viral infection, acute inflammation of the lung, or interstitial lung disease.

### Embodiment 138

The use of Embodiment 137 for treating an inflammatory lung condition or disease caused by a viral infection in a patient in need thereof.

### Embodiment 139

The use of Embodiment 138, wherein the viral infection is a human coronavirus infection, an influenza virus infection, or a HIV virus infection.

### Embodiment 140

The use of Embodiment 139, wherein the human coronavirus is HCOV 229E, HCOV OC43, HCOV NL63, HCOV HKU1, SARS COV, MERS CoV, or SARS COV-2, or a mutated strain thereof.

### Embodiment 141

The use of Embodiment 140, wherein the human coronavirus is SARS COV-2.

### Embodiment 142

The use of Embodiment 141, wherein the human coronavirus is a mutated strain of SARS COV 2.

### Embodiment 143

The use of any one of Embodiments 138-142, wherein the inflammatory lung condition or disease caused by the viral infection is hypercytokinaemia, haemophagocytic lymphohistiocytosis, pneumonia, acute respiratory distress syndrome, or systemic inflammatory response syndrome.

### Embodiment 144

The use of Embodiment 143, wherein the inflammatory lung condition or disease caused by the viral infection is acute respiratory distress syndrome.

### Embodiment 145

The use of Embodiment 137 for treating acute inflammation of the lung in a patient in need thereof.

### Embodiment 146

The use of Embodiment 145, wherein the acute inflammation of the lung is caused by a bacterial infection.

### Embodiment 147

The use of Embodiment 145, wherein the acute inflammation of the lung is pneumonia or acute respiratory distress syndrome.

### Embodiment 148

The use of Embodiment 137 for treating interstitial lung disease in a patient in need thereof.

### Embodiment 149

The use of Embodiment 137, wherein the interstitial lung disease is idiopathic pulmonary fibrosis.

### Embodiment 150

The use of Embodiment 121, wherein the liver disease or disorder is NASH or NAFLD.

### Embodiment 151

A compound that is (Z)-5-(4-(2-(5-(1-hydroxyethyl)pyridin-2-yl)ethoxy)benzylidene)thiazolidine-2,4-dione, or a pharmaceutically acceptable salt thereof.

### Embodiment 152

A compound that is 2,2′-disulfanediylbis(3-(4-(2-(5-(1-hydroxyethyl)pyridin-2-yl)ethoxy)phenyl) propanoic acid), or a pharmaceutically acceptable salt thereof.

### Embodiment 153

A compound that is 2-((1-hydroxy-3-(4-(2-(5-(1-hydroxyethyl)pyridin-2-yl)ethoxy)phenyl) propan-2-yl)disulfaneyl)-3-(4-(2-(5-(1-hydroxyethyl)pyridin-2-yl)ethoxy)phenyl) propanoic acid, or a pharmaceutically acceptable salt thereof.

### Embodiment 154

A compound that is 5-(4-(2-(5-(1-hydroxyethyl)pyridin-2-yl)ethoxy)benzyl) thiazolidin-2-one, or a pharmaceutically acceptable salt thereof.

### Embodiment 155

The compound of any one of Embodiments 151-154, wherein the pharmaceutically acceptable salt is the hydrochloride salt.

### Embodiment 156

A composition comprising 99.0 to 99.9 wt/wt % of leriglitazone (free base), leriglitazone hydrochloride, or leriglitazone hydrate, and:
(i) 0.0001 to 0.2 wt/wt % of (Z)-5-(4-(2-(5-(1-hydroxyethyl)pyridin-2-yl)ethoxy)benzylidene)thiazolidine-2,4-dione, or the hydrochloride salt thereof;
(ii) 0.0001 to 0.2 wt/wt % of 2,2′-disulfanediylbis(3-(4-(2-(5-(1-hydroxyethyl)pyridin-2-yl)ethoxy)phenyl) propanoic acid), or the hydrochloride salt thereof;
(iii) 0.0001 to 0.2 wt/wt % of 2-((1-hydroxy-3-(4-(2-(5-(1-hydroxyethyl)pyridin-2-yl)ethoxy)phenyl) propan-2-yl)disulfaneyl)-3-(4-(2-(5-(1-hydroxyethyl)pyridin-2-yl)ethoxy)phenyl) propanoic acid, or the hydrochloride salt thereof; and/or
(iv) 0.15 wt/wt % of 5-(4-(2-(5-(1-hydroxyethyl)pyridin-2-yl)ethoxy)benzyl) thiazolidin-2-one, or the hydrochloride salt thereof.

### Embodiment 157

The composition of Embodiment 156 comprising 99.0 to 99.9 wt/wt % of leriglitazone (free base), leriglitazone hydrochloride, or leriglitazone hydrate, and 0.0001 to 0.15 wt/wt % of (Z)-5-(4-(2-(5-(1-hydroxyethyl)pyridin-2-yl)ethoxy)benzylidene)thiazolidine-2,4-dione, or the hydrochloride salt thereof.

### Embodiment 158

The composition of Embodiments 156 or 157 comprising 99.0 to 99.9 wt/wt % of leriglitazone (free base), leriglitazone hydrochloride, or leriglitazone hydrate, and 0.0001 to 0.15 wt/wt % of 2,2′-disulfanediylbis(3-(4-(2-(5-(1-hydroxyethyl)pyridin-2-yl)ethoxy)phenyl) propanoic acid), or the hydrochloride salt thereof.

### Embodiment 159

The composition of any one of Embodiments 156-158 comprising 99.0 to 99.9 wt/wt % of leriglitazone (free base), leriglitazone hydrochloride, or leriglitazone hydrate, and 0.0001 to 0.15 wt/wt % of 2-((1-hydroxy-3-(4-(2-(5-(1-hydroxyethyl)pyridin-2-yl)ethoxy)phenyl) propan-2-yl)disulfaneyl)-3-(4-(2-(5-(1-hydroxyethyl)pyridin-2-yl)ethoxy)phenyl) propanoic acid, or the hydrochloride salt thereof.

### Embodiment 160

The composition of any one of Embodiments 156-159 comprising 99.0 to 99.9 wt/wt % of leriglitazone (free base), leriglitazone hydrochloride, or leriglitazone hydrate, and 0.0001 to 0.15 wt/wt % of 5-(4-(2-(5-(1-hydroxyethyl)pyridin-2-yl)ethoxy)benzyl) thiazolidin-2-one, or the hydrochloride salt thereof.

### Embodiment 161

The composition of any one of Embodiments 156-160 further comprising water and, optionally, one or more pharmaceutically acceptable carriers, diluents or excipients.

### Embodiment 162

A pharmaceutical formulation comprising:
(a) composition of any one of Embodiments 156-160 in an amount from 0.01% w/v to 5% w/v;
(b) one or more cellulose derivatives in an amount from 0.01% w/v to 2% w/v, wherein the one or more cellulose derivatives have a viscosity between 1500 and 4500 mPa*s, preferably between 1500 to 3500 mPa*s, more preferably between 1500 and 3000 mPa*s as measured by Brookfield viscosity method; and
(c) water QS to 100%.

### Embodiment 163

The pharmaceutical formulation according to Embodiment 162, wherein composition is present in an amount from 1% w/v to 2% w/v.

### Embodiment 164

The pharmaceutical formulation according to Embodiment 162 or 163, wherein the one or more cellulose derivatives comprise microcrystalline cellulose, carboxymethylcellulose sodium, or mixtures thereof.

### Embodiment 165

The pharmaceutical formulation according to any one of Embodiments 162-164 further comprising one or more pH regulators, e.g., sodium citrate, citric acid monohydrate, or mixtures thereof.

### Embodiment 166

The pharmaceutical formulation according to any one of Embodiments 162-165 further comprising one or more preservatives in an amount from 0.001% w/v to 1.0% w/v.

### Embodiment 167

The pharmaceutical formulation according to Embodiment 16, wherein the one or more preservatives comprises sodium benzoate.

### Embodiment 168

The pharmaceutical formulation according to any one of Embodiments 12-17 further comprising one or more flavouring agents in an amount of 0.001% w/v to 3% w/v.

### Embodiment 169

The pharmaceutical formulation according to Embodiment 168, wherein the one or more flavouring agents comprise strawberry flavor.

### Embodiment 170

The pharmaceutical formulation according to any one of Embodiments 162-169 further comprising one or more sweetening agents in an amount of 0.01% w/v to 15% w/v.

### Embodiment 171

The pharmaceutical formulation according to Embodiment 170, wherein the sweetening agent is sorbitol, saccharin sodium, or mixtures thereof.

### Embodiment 172

The pharmaceutical formulation according to any one of Embodiments 162-171, wherein the pH of the formulation is of 3.5 to 4.5.

### Embodiment 173

The pharmaceutical formulation according to Embodiment 172, wherein the pH of the formulation is about 4.

### Embodiment 174

The pharmaceutical formulation according to any one of Embodiments 162-173, wherein the viscosity of the formulation is higher than 50 mPa*s and less than 5000 mPa*s, preferably less than 4,500 mPa*s.

### Embodiment 175

The pharmaceutical formulation of Embodiment 162 comprising:
(a) the composition in an amount of 1.0% to 2.0% w/v;
(b) about 1% w/v of microcrystalline cellulose;
(c) about 0.5% w/v of carboxymethyl cellulose sodium;
(d) water QS to 100%;
and, optionally;
(e) about 8% w/v sorbitol powder;
(f) about 0.05% w/v saccharin sodium;
(g) about 0.1% w/v sodium benzoate;
(h) about 0.5% w/v sodium citrate;
(i) about 0.1% w/v citric acid monohydrate;
(j) about 0.015% w/v strawberry flavour;
(k) about 1% w/v 0.2 M aqueous citric acid monohydrate solution.

### Embodiment 176

A pharmaceutical formulation prepared by admixing:
(a) about 1.5% w/v the composition of any one of Embodiments 156-160;
(b) about 8% w/v sorbitol powder;
(c) about 1% w/v microcrystalline cellulose;
(d) about 0.5% w/v carboxymethyl cellulose sodium;
(e) about 0.05% w/v saccharin sodium;
(f) about 0.1% w/v sodium benzoate;
(g) about 0.5% w/v sodium citrate;
(h) about 0.1% w/v citric acid monohydrate;
(i) about 0.015% w/v strawberry flavour;
(j) about 1% w/v 0.2 M aqueous citric acid monohydrate solution; and
(k) water QS to 100%.

### Embodiment 177

The pharmaceutical formulation of any one of Embodiments 162-176 for use in treating a disease or disorder in a patient in need thereof, wherein the disease or disorder is a central nervous system disease or disorder, mitochondrial disease, a liver disease or disorder, chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, an inflammatory respiratory disease, or a lung disease or disorder.

### Embodiment 178

The pharmaceutical formulation of Embodiment 177, wherein the disease or disorder is a central nervous system disease or disorder.

### Embodiment 179

The pharmaceutical formulation of Embodiment 178, wherein the central nervous system disease or disorder is selected from the group consisting of neurodegenerative disease, cerebrovascular disease, seizure, epilepsy, viral disease, neuroinflammatory disease, brain tumor, organic acidemias, fatty acid disorder, and genetic mitochondrial disorder.

### Embodiment 180

The pharmaceutical formulation of Embodiment 179, wherein the central nervous system disease or disorder is a neurodegenerative disease.

### Embodiment 181

The pharmaceutical formulation of Embodiment 180, wherein the neurodegenerative disease is selected from the group consisting of Alzheimer's disease, Huntington's chorea, Parkinson's disease, multiple sclerosis, neuromyelitis optica, leukodystrophy, amyotrophic lateral sclerosis (ALS), degenerative ataxia, multiple system atrophy, neurodegeneration and brain iron accumulation disorders (NBIA), neuromyopathy, and a motor neuron disease.

### Embodiment 182

The pharmaceutical formulation of Embodiment 181, wherein the leukodystrophy is X-linked adrenoleukodystrophy, adrenomyeloneuropathy, cerebral adrenoleukodystrophy, or metachromatic leukodystrophy.

### Embodiment 183

The pharmaceutical formulation of Embodiment 182, wherein the leukodystrophy is cerebral adrenoleukodystrophy.

### Embodiment 184

The pharmaceutical formulation of Embodiment 181, wherein the degenerative ataxia is Friedreich's ataxia.

### Embodiment 185

The pharmaceutical formulation of Embodiment 181, wherein the motor neuron disease is selected from the group consisting of progressive bulbar palsy, pseudobulbar palsy, primary lateral sclerosis (PLS), progressive muscular atrophy, spinal muscular atrophy (SMA), post-polio syndrome (PPS)-Marie-Tooth disease, Guillan-Barré syndrome, and adrenomyeloneuropathy (AMN).

### Embodiment 186

The pharmaceutical formulation of Embodiment 179, wherein the cerebrovascular disease is selected from the group consisting of global or local ischemia, intracerebral haemorrhage, stroke, and vascular dementia.

### Embodiment 187

The pharmaceutical formulation of Embodiment 179, the viral disease is selected from the group consisting of meningitis, encephalitis, rabies, measles, mumps, poliomyelitis, herpes simplex, and varicella zoster.

### Embodiment 188

The pharmaceutical formulation of Embodiment 178, wherein the central nervous system disease or disorder is a rare metabolic disease selected from the group consisting of organic acidemias, fatty acid disorders and genetic mitochondrial disorders.

### Embodiment 189

pharmaceutical formulation of Embodiment 177, wherein the disease or disorder is a mitochondrial disease.

### Embodiment 190

The pharmaceutical formulation of Embodiment 189, wherein the mitochondrial disease is a primary mitochondrial disorder selected from the group consisting of Rett syndrome, Alper's disease; Leber's hereditary optic neuropathy (LHON); Kearns-Sayre syndrome (KSS); Leigh's syndrome; Leigh-like syndrome; maternally inherited Leigh syndrome (MILS); mitochondrial depletion syndrome (MDS); mitochondrial DNA depletion syndrome (MDDS); mitochondrial encephalomyopathy; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; chronic progressive external opthalmoplegia (CPEO); dominant optic atrophy (DOA); autosomal dominant optic atrophy (ADOA); mitochondrial myopathy; cardiomyopathy; mitochondrial encephalopathy; myoclonic epilepsy; maternally inherited diabetes and deafness (MIDD); ataxia neuropathy spectrum; 3-methylglutaconic aciduria; sensoneural deafness; neuroradiological findings of Leigh-like syndrome (MEGDEL); SURF1 (COX definient Leigh syndrome due to complex IV surfeit protein deficiency); oxidative phosphorylation disorders; Berth syndrome; lethal infantile cardiomyopathy (LIC); pyruvate carboxylase deficiency; pyruvate dehydrogenase deficiency; POLG mutation; isolated or combined OXPHOS deficiencies with so far unsolved genetic defect including disturbed pyruvate oxidation and ATP plus PCr production rates; POLG2 mutation; carnitine-acyl-cartinine deficiency; carnitine deficiency; creatinine deficiency syndromes; Co-Enzyme Q10 deficiency; Complex I deficiency; Complex II deficiency; Complex III deficiency; Complex IV deficiency; Complex V deficiency; lactic acidosis; leukoencephalopathy with brain stem and spinal cord involvement and lactate elevation (LBSL); Luft disease; carnitine palmitoyltransferase (CPT I or CPT II) deficiency; short-chain acyl-CoA dehydrogenase deficiency (SCAD); short-chain 3-hydroxyacetyl-CoA dehydrogenase deficiency (SCHAD); medium-chain acyl-CoA dehydrogenase deficiency (MCAD); multiple acyl-CoA dehydrogenase deficiency (MADD); long-chain acyl-CoA dehydrogenase deficiency (LCAD); very long-chain acyl-CoA dehydrogenase deficiency (VLCAD); trifunctional protein (TFP) deficiency; and glutaric aciduria Type II.

### Embodiment 191

The pharmaceutical formulation of Embodiment 190, wherein the mitochondrial disease is selected from the group consisting of Rett syndrome; dominant optic atrophy (DOA); autosomal dominant optic atrophy (ADOA); Complex I deficiency; Leber hereditary optic neuropathy (LHON); Kearns-Sayre syndrome (KSS); Leigh's syndrome; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; and chronic progressive external opthalmoplegia (CPEO).

### Embodiment 192

The pharmaceutical formulation of Embodiment 189, wherein the mitochondrial disease is a secondary mitochondrial disorder selected from the group consisting of Duchenne muscular dystrophy (DMD); Becker muscular dystrophy (BMD); myotonic dystrophy (BMD); congenital myopathies; glycogen storage disorders; spinal-bulbar muscular atrophy (SBMA); argininosuccinic aciduria; autism spectrum disorder (ASD); autoimmune diseases of the skin (such as pemphigus vulgaris and lupus); methylmalonic and propionic acidurias; disorders or purine and/or pyrimidine synthesis; facioscapulohumeral muscular dystrophy (FSHD); congenital muscular dystrophies; collagen VI muscular dystrophies (e.g., Ullrich congenital muscular dystrophy, Bethlem myopathy, oculopharyngeal distal and Emery-Dreifuss); DiGeorge syndrome; and neuromuscular disorders (such as limb-girdle muscular dystrophy, inflammatory myopathies, Charcot Marie Tooth (CMT) neuropathy, and drug-induced peripheral neuropathies).

### Embodiment 193

The pharmaceutical formulation of Embodiment 177, wherein the lung disease or disorder is an inflammatory lung condition or disease caused by a viral infection, acute inflammation of the lung, or interstitial lung disease.

### Embodiment 194

The pharmaceutical formulation of Embodiment 193 for treating an inflammatory lung condition or disease caused by a viral infection in a patient in need thereof.

### Embodiment 195

The pharmaceutical formulation of Embodiment 194, wherein the viral infection is a human coronavirus infection, an influenza virus infection, or a HIV virus infection.

### Embodiment 196

The pharmaceutical formulation of Embodiment 195, wherein the human coronavirus is HCOV 229E, HCOV OC43, HCOV NL63, HCOV HKU1, SARS COV, MERS CoV, or SARS COV-2, or a mutated strain thereof.

### Embodiment 197

The pharmaceutical formulation of Embodiment 196, wherein the human coronavirus is SARS COV-2.

### Embodiment 198

The pharmaceutical formulation of Embodiment 197, wherein the human coronavirus is a mutated strain of SARS COV 2.

### Embodiment 199

The pharmaceutical formulation of any one of Embodiments 194-198, wherein the inflammatory lung condition or disease caused by the viral infection is hypercytokinaemia, haemophagocytic lymphohistiocytosis, pneumonia, acute respiratory distress syndrome, or systemic inflammatory response syndrome.

### Embodiment 200

The pharmaceutical formulation of Embodiment 199, wherein the inflammatory lung condition or disease caused by the viral infection is acute respiratory distress syndrome.

### Embodiment 201

The pharmaceutical formulation of Embodiment 193 for treating acute inflammation of the lung in a patient in need thereof.

### Embodiment 202

The pharmaceutical formulation of Embodiment 201, wherein the acute inflammation of the lung is caused by a bacterial infection.

### Embodiment 203

The pharmaceutical formulation of Embodiment 201, wherein the acute inflammation of the lung is pneumonia or acute respiratory distress syndrome.

### Embodiment 204

The pharmaceutical formulation of Embodiment 203 for treating interstitial lung disease in a patient in need thereof.

### Embodiment 205

The pharmaceutical formulation of Embodiment 203, wherein the interstitial lung disease is idiopathic pulmonary fibrosis.

### Embodiment 206

The pharmaceutical formulation of Embodiment 177, wherein the liver disease or disorder is nonalcoholic steatohepatitis (NASH) or nonalcoholic fatty liver disease (NAFLD).

### Embodiment 207

The composition of any one of Embodiments 156-161 comprising leriglitazone hydrochloride Form A.

### Embodiment 208

The composition of any one of Embodiments 156-161 comprising leriglitazone Form B.

### Embodiment 209

The composition of any one of Embodiments 156-161 comprising leriglitazone hydrate Form C.

### Embodiment 210

The pharmaceutical formulation of any one of Embodiments 162-206 comprising leriglitazone hydrochloride Form A.

### Embodiment 211

The pharmaceutical formulation of any one of Embodiments 162-206 comprising leriglitazone Form B.

### Embodiment 212

The pharmaceutical formulation of any one of Embodiments 162-206 comprising leriglitazone hydrate Form C.

### Embodiment 213

A kit comprising the pharmaceutical formulation of any one of Embodiments 162-206 or 210-212 in a container and (ii) a label with instructions how to use the kit.

### Embodiment 214

The kit of Embodiment 213, wherein the label is approved by the United States Food and Drug Administration (FDA), the European Medicines Agency (EMA), the China Food and Drug Administration (CFDA), or the Japanese Ministry of Health Labor and Welfare (MHLW).

### Embodiment 215

The kit of claims Embodiment 213 or Embodiment 214, further comprising a graduated oral syringe of 5 mL, 10 mL, 12 mL or 15 mL.

### EXAMPLES

### Instrumentation

### X-Ray Powder Diffraction (XRPD)

Sample preparation: In order to acquire a powder diffraction pattern of the obtained solid, approximately 20 mg of the non-manipulated samples were prepared in standard sample holders using two foils of polyacetate.

Data collection: Powder diffraction patterns were acquired on a Bruker D8 Advance Series 2Theta/Theta powder diffraction system using CuKal-radiation (1.54060 Å) in transmission geometry. The system is equipped with a VĂNTEC-1 single photon counting PSD, a Germanium monochromator, a ninety positions auto changer sample stage, fixed divergence slits and radial soller. Programs used: Data collection with DIFFRAC plus XRD Commander V.2.4.1 and evaluation with EVA V.12.0.

### Differential Scanning Calorimetry (DSC)

DSC analyses were recorded in a Mettler Toledo DSC822e. Samples of 1-3 mg were weighed (using a microscale MX5, Mettler) into 40 μL aluminium crucibles with a pinhole lid, and were heated, under nitrogen flow (50 mL/min), from 30 to 300° C. at a heating rate of 10° C./min. Data collection and evaluation was done with STARe software.

### Thermogravimetric Analysis (TGA)

Thermogravimetric analyses were recorded in a Mettler Toledo SDTA851e. Samples of 1-3 mg were weighed (using a microscale MX5, Mettler) into 40 μL aluminium crucibles with a pinhole lid, and were heated at 10° C./min between 3° and 300° C., under nitrogen flow (50 mL/min). Data collection and evaluation was done with STARe software.

### FT-Raman Spectroscopy

Data acquisition: FT-Raman measurements were carried out on the Raman Module of the Thermo Nicolet iS50 spectrometer equipped with an InGaAs detector, CaF2 beamsplitter and a laser operating at 1064 nm.

Sample preparation: Approximately 5 mL of the syrup were centrifuged in a Genevac, then the solid at the bottom was scooped with a spatula and analysed.

### Single Crystal Structure Determination

Data collection: The measured crystals were prepared under inert conditions immersed in perfluoropolyether as protecting oil for manipulation. Crystal structure determinations were carried out using a Apex DUO Kappa 4-axis goniometer equipped with an APPEX 2 4K CCD area detector, a Microfocus Source E025 IuS using MoK_{α} radiation (0.71073 Å), Quazar MX multilayer Optics as monochromator and an Oxford Cryosystems low temperature device Cryostream 700 plus (T=−173° C.). Full-sphere data collection was used with ω and φ scans.

Programs used: Data collection APEX-2, data reduction Bruker Saint and absorption correction SADABS.

Structure Solution and Refinement: Crystal structure solution was achieved using the computer program SHELXT. Visualization was performed with the program SHELXle. Missing atoms were subsequently located from difference Fourier synthesis and added to the atom list. Least-squares refinement on F² using all measured intensities was carried out using the program SHELXL 2015. All non-hydrogen atoms were refined including anisotropic displacement parameters.

### Example 1

### Characterization of Form A

Leriglitazone hydrochloride Form A was prepared by suspending leriglitazone hydrochloride in ethanol at room temperature for 10 days, decanting the mother liquor, and collecting the solid thus obtained.

The X-ray powder diffraction (XRPD) diffractogram of Form A is shown in FIG. 1. The XRPD peak list (±0.2 degrees 2θ) is provided in Table 1.

TABLE 1

| | d-spacing | |
|---|---|---|
| degrees 2Θ | (angstrom) | Relative Intensity |
| 8.8 | 10.03 | 5.1 |
| 9.2 | 9.60 | 3.0 |
| 10.8 | 8.22 | 11.6 |
| 10.8 | 8.22 | 11.6 |
| 12.4 | 7.15 | 12.9 |
| 12.7 | 6.98 | 3.9 |
| 15.5 | 5.72 | 5.3 |
| 16.5 | 5.37 | 5.8 |
| 17.0 | 5.22 | 20.0 |
| 17.4 | 5.10 | 5.3 |
| 17.7 | 4.99 | 0.4 |
| 18.5 | 4.79 | 18.1 |
| 19.7 | 4.50 | 21.1 |
| 20.0 | 4.44 | 0.7 |
| 20.5 | 4.32 | 57.1 |
| 20.8 | 4.27 | 10.8 |
| 21.7 | 4.10 | 100.0 |
| 22.4 | 3.96 | 9.0 |
| 22.7 | 3.91 | 1.9 |
| 23.0 | 3.87 | 1.9 |
| 24.3 | 3.65 | 1.2 |
| 24.9 | 3.57 | 0.7 |
| 25.2 | 3.53 | 1.1 |
| 25.5 | 3.49 | 10.7 |
| 26.1 | 3.41 | 41.9 |
| 26.7 | 3.34 | 7.5 |
| 27.0 | 3.29 | 21.7 |
| 27.8 | 3.21 | 7.9 |
| 28.3 | 3.16 | 3.8 |
| 28.7 | 3.11 | 0.9 |
| 29.3 | 3.04 | 13.4 |
| 29.7 | 3.01 | 6.2 |
| 29.9 | 2.99 | 3.1 |
| 30.1 | 2.96 | 2.7 |
| 31.2 | 2.87 | 1.4 |
| 31.7 | 2.82 | 1.2 |
| 31.9 | 2.81 | 0.8 |
| 32.3 | 2.77 | 2.3 |
| 32.7 | 2.74 | 14.1 |
| 33.2 | 2.69 | 1.2 |
| 33.5 | 2.67 | 2.4 |
| 33.8 | 2.65 | 1.3 |
| 34.1 | 2.63 | 0.2 |
| 34.3 | 2.61 | 0.7 |
| 35.0 | 2.56 | 0.2 |
| 35.2 | 2.54 | 0.4 |
| 35.6 | 2.52 | 3.1 |
| 35.9 | 2.50 | 1.9 |
| 36.4 | 2.46 | 2.7 |
| 37.1 | 2.42 | 0.9 |
| 37.5 | 2.40 | 2.9 |
| 37.9 | 2.37 | 4.3 |
| 38.2 | 2.35 | 1.8 |
| 38.7 | 2.33 | 0.4 |
| 39.0 | 2.31 | 0.3 |
| 39.7 | 2.27 | 1.6 |
| 40.3 | 2.24 | 4.0 |

The Raman spectrum of Form A is shown in FIG. 2. The Raman peak list (±4 cm⁻¹) is provided in Table 2.

TABLE 2

| Raman shift (cm⁻¹) | Relative Intensity |
|---|---|
| 3307 | medium |
| 3096 | medium |
| 3065 | strong |
| 2970 | strong |
| 2925 | strong |
| 2871 | strong |
| 1748 | strong |
| 1685 | medium |
| 1642 | strong |
| 1612 | strong |
| 1585 | medium |
| 1436 | medium |
| 1327 | medium |
| 1310 | medium |
| 1244 | strong |
| 1207 | strong |
| 1184 | medium |
| 1153 | medium |
| 1088 | medium |
| 1070 | medium |
| 1034 | medium |
| 936 | medium |
| 908 | medium |
| 870 | strong |
| 854 | strong |
| 789 | medium |
| 737 | medium |
| 707 | medium |
| 646 | strong |
| 638 | strong |
| 605 | strong |
| 497 | medium |
| 469 | medium |
| 437 | medium |
| 380 | medium |
| 333 | medium |
| 309 | medium |
| 221 | medium |
| 116 | strong |

The structure of Form A was solved by single crystal X-ray diffraction. Selected crystals of Form A were inspected via optical microscopy using polarized light. A suitable single crystal, which was obtained by crystallization in ethanol 90% was prepared coated in perfluoropolyether oil and mounted using a magnetic kapton loop for SCXRD determination. The measurement was performed at 100 K.

After structure solution from the data collected a structure of excellent quality could be refined (R1: 4.83%). The asymmetric unit contains one molecule of the cationic organic compound which is partially disordered and one a chloride anion, confirming the identity of the compound as a hydrochloride salt.

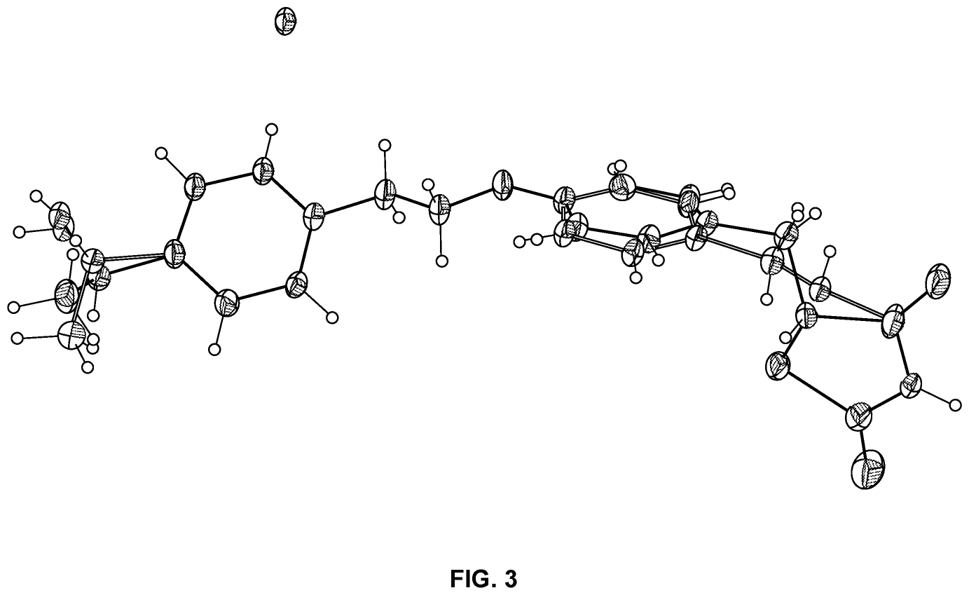

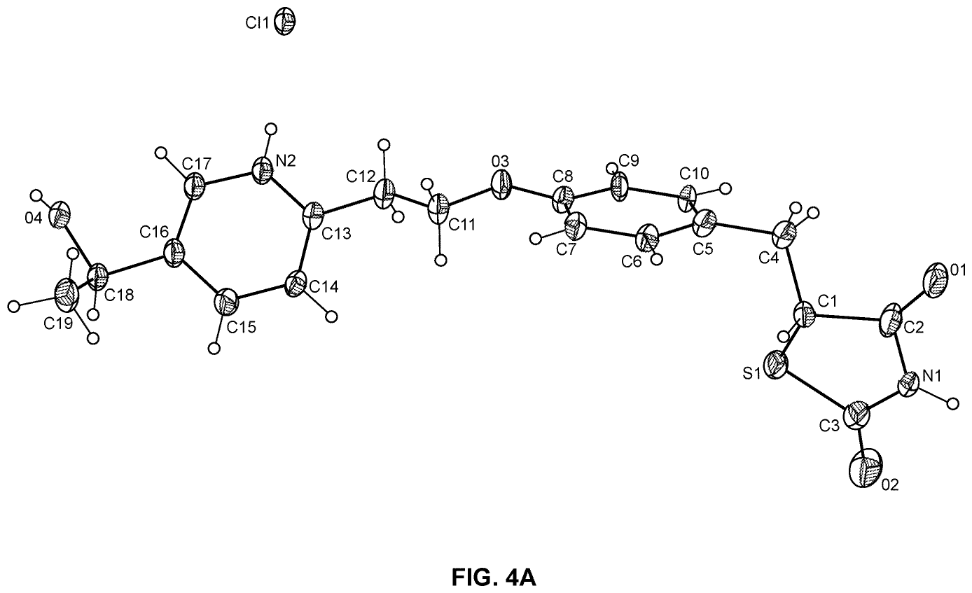

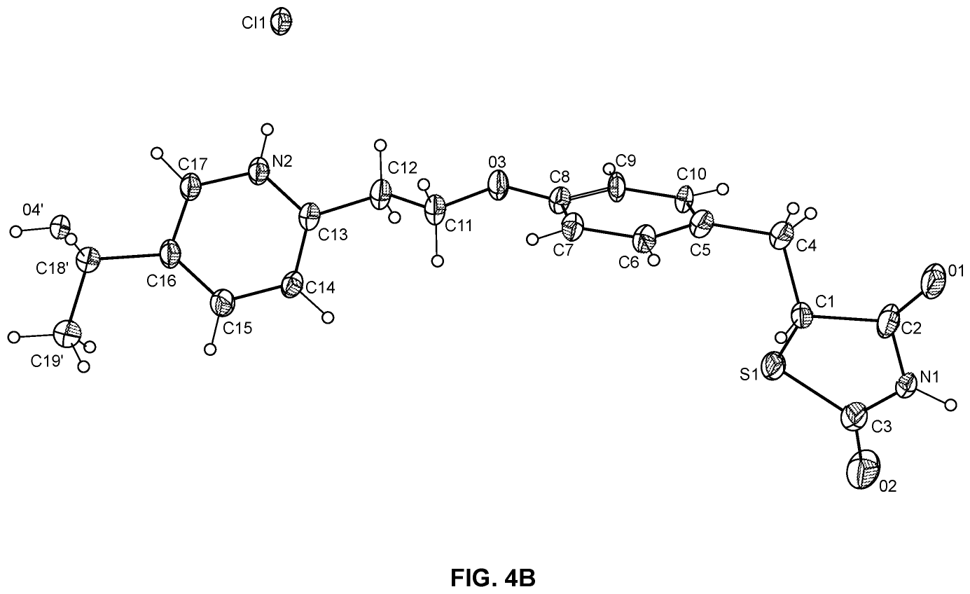

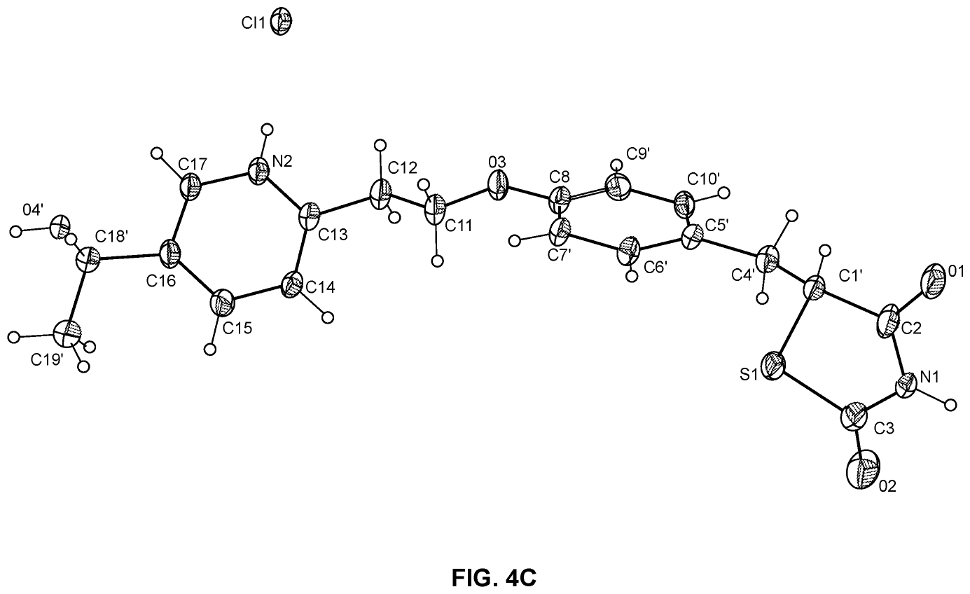

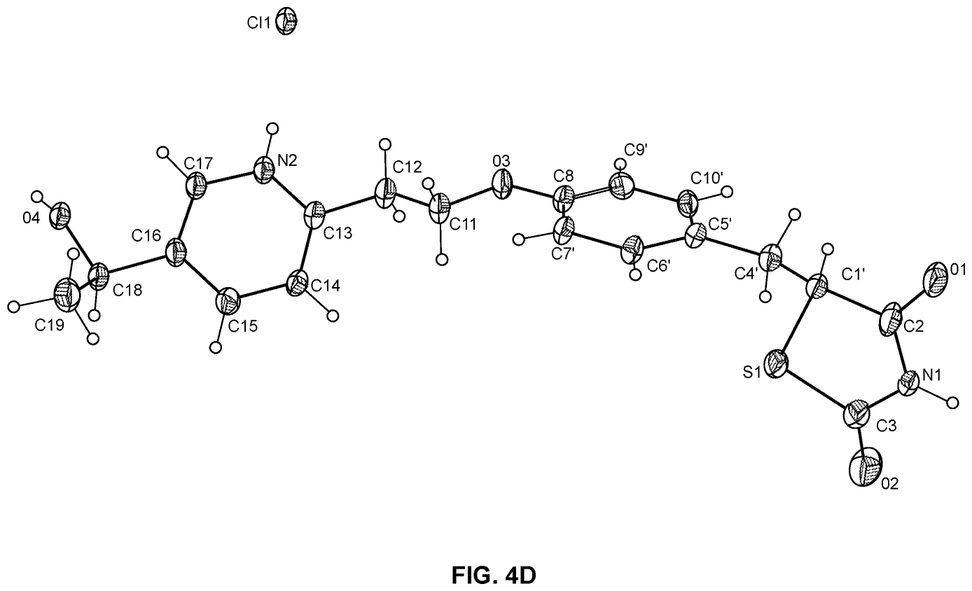

The disorder observed in the cationic organic compound confirms the presence of a combination/mixture of the different enantiomeric and diastereoisomeric forms in one single crystal. A pure enantiomeric substance should crystallize always in a chiral space group. In the case the mixture formed by four molecules including two diastereoisomeric forms and its corresponding enantiomers crystallizes exceptionally in a centrosymmetric space group with all four molecules disordered sharing the same position in the crystal packing. See FIG. 3 and FIGS. 4a-4d. The four stereoisomers are so similar in its molecule volume that they crystallize together mixed in one single crystal form. The occupancy ratios are 0.72:0.28 for the chiral center with the alcohol group and 0.52:0.48 for the chiral center attached to the sulfur atom. The exact ratio of stereoisomers cannot be assigned since it is not clear which ratio of the first chiral group corresponds to the second chiral group but it should be around 70:30. Also it should be remarked that the ratios obtained are only representative for the crystal measured and could vary by structure determination of another crystals.

Form A melts with an onset temperature of about 203.98° C. and a peak temperature of about 211.87° C. based on DSC analysis. See FIG. 5.

### Example 2

### Characterization of Form B

Leriglitazone Form B, i.e., the unsolvated free base, was prepared by suspending leriglitazone hydrochloride Form A in water at 80° C., and then cooling down to room temperature and stirring overnight. Afterwards, the white solid was filtered off and dried at 100° C. under vacuum of 2 mbar for 15 hours.

The X-ray powder diffraction (XRPD) diffractogram of Form B is shown in FIG. 6. The XRPD peak list (±0.2 degrees 2θ) is provided in Table 3.

TABLE 3

| | d-spacing | |
|---|---|---|
| degrees 2Θ | (angstrom) | Relative Intensity |
| 7.6 | 11.64 | 7.1 |
| 9.0 | 9.78 | 22.9 |
| 10.5 | 8.43 | 14.2 |
| 12.4 | 7.11 | 9.6 |
| 15.2 | 5.84 | 25.5 |
| 16.1 | 5.49 | 5.3 |
| 16.6 | 5.33 | 33.8 |
| 18.5 | 4.79 | 4.0 |
| 18.8 | 4.73 | 83.1 |
| 19.3 | 4.61 | 4.2 |
| 19.8 | 4.48 | 16.0 |
| 20.1 | 4.41 | 11.9 |
| 20.5 | 4.33 | 2.0 |
| 21.1 | 4.21 | 100.0 |
| 21.8 | 4.07 | 27.8 |
| 22.9 | 3.89 | 5.2 |
| 23.2 | 3.83 | 4.9 |
| 23.5 | 3.79 | 8.4 |
| 23.8 | 3.73 | 58.2 |
| 24.7 | 3.60 | 42.6 |
| 25.4 | 3.50 | 27.5 |
| 26.0 | 3.42 | 7.2 |
| 26.5 | 3.36 | 6.1 |
| 26.9 | 3.31 | 11.2 |
| 27.4 | 3.26 | 5.1 |
| 28.0 | 3.18 | 23.7 |
| 28.9 | 3.09 | 6.7 |
| 29.1 | 3.06 | 8.3 |
| 29.5 | 3.03 | 5.1 |
| 29.9 | 2.99 | 2.1 |
| 30.8 | 2.90 | 22.6 |
| 31.2 | 2.86 | 3.5 |
| 31.5 | 2.83 | 2.6 |
| 32.1 | 2.79 | 3.3 |
| 32.6 | 2.74 | 2.6 |
| 32.9 | 2.72 | 1.8 |
| 33.7 | 2.65 | 2.3 |
| 34.4 | 2.60 | 4.8 |
| 35.4 | 2.53 | 1.7 |
| 35.8 | 2.51 | 2.3 |
| 36.3 | 2.48 | 1.7 |
| 36.9 | 2.43 | 1.1 |
| 37.1 | 2.42 | 0.6 |
| 38.0 | 2.37 | 3.5 |
| 38.4 | 2.34 | 2.5 |
| 39.3 | 2.29 | 3.5 |

Form B melts with an onset temperature of about 149.34° C. and a peak temperature of about 153.56° C. based on DSC analysis. Thermogravimetric analysis shows a 0.9% loss of mass between 40° C. and 100° C., suggesting that it is desolvated. See FIG. 7.

### Example 3

### Characterization of Form C

Leriglitazone hydrate Form C was prepared by suspending leriglitazone hydrochloride Form A in water at room temperature for 10 days, decanting the mother liquor, and collecting the solid thus obtained.

The X-ray powder diffraction (XRPD) diffractogram of Form C is shown in FIG. 8. The XRPD peak list (±0.2 degrees 2θ) is provided in Table 4.

TABLE 4

| | d-spacing | |
|---|---|---|
| degrees 2Θ | (angstrom) | Relative Intensity |
| 4.0 | 22.07 | 1.1 |
| 6.2 | 14.14 | 4.6 |
| 7.7 | 11.41 | 20.7 |
| 8.9 | 9.97 | 1.9 |
| 9.2 | 9.60 | 16.0 |
| 9.9 | 8.91 | 1.0 |
| 10.6 | 8.32 | 16.1 |
| 12.7 | 6.99 | 31.3 |
| 15.4 | 5.74 | 35.3 |
| 16.5 | 5.36 | 7.3 |
| 16.9 | 5.24 | 29.1 |
| 18.4 | 4.82 | 2.4 |
| 19.1 | 4.65 | 100.0 |
| 19.4 | 4.57 | 5.1 |
| 19.8 | 4.48 | 14.9 |
| 20.4 | 4.36 | 13.5 |
| 21.0 | 4.23 | 70.2 |
| 21.3 | 4.17 | 26.6 |
| 21.6 | 4.11 | 31.1 |
| 22.1 | 4.02 | 1.1 |
| 23.0 | 3.86 | 4.7 |
| 23.3 | 3.82 | 14.5 |
| 23.8 | 3.73 | 74.2 |
| 24.6 | 3.62 | 23.1 |
| 24.9 | 3.57 | 17.4 |
| 25.4 | 3.51 | 12.8 |
| 25.6 | 3.47 | 7.4 |
| 25.9 | 3.44 | 42.7 |
| 26.4 | 3.37 | 5.6 |
| 27.3 | 3.26 | 20.3 |
| 28.0 | 3.18 | 23.7 |
| 28.8 | 3.10 | 3.4 |
| 29.6 | 3.02 | 15.8 |
| 30.1 | 2.97 | 6.7 |
| 30.7 | 2.91 | 5.8 |
| 30.9 | 2.89 | 13.4 |
| 31.2 | 2.86 | 4.5 |
| 31.5 | 2.84 | 6.4 |
| 31.9 | 2.81 | 9.5 |
| 32.6 | 2.75 | 3.4 |
| 33.0 | 2.71 | 4.1 |
| 33.5 | 2.68 | 1.0 |
| 33.8 | 2.65 | 0.5 |
| 34.2 | 2.62 | 2.2 |
| 34.5 | 2.60 | 2.3 |
| 34.8 | 2.57 | 0.7 |
| 35.3 | 2.54 | 2.5 |
| 35.6 | 2.52 | 1.9 |
| 36.1 | 2.49 | 1.3 |
| 36.4 | 2.47 | 4.8 |
| 37.1 | 2.42 | 1.0 |
| 37.7 | 2.39 | 1.1 |
| 38.2 | 2.36 | 3.9 |
| 38.7 | 2.33 | 3.2 |
| 38.9 | 2.31 | 2.1 |
| 39.3 | 2.29 | 0.9 |
| 39.8 | 2.26 | 0.7 |

The Raman spectrum of Form C is shown in FIG. 9. The Raman peak list (±4 cm⁻¹) is provided in Table 5.

TABLE 5

| Raman shift (cm⁻¹) | Relative Intensity |
|---|---|
| 3056 | strong |
| 3017 | strong |
| 2992 | strong |
| 2960 | strong |
| 2921 | strong |
| 1735 | medium |
| 1608 | strong |
| 1582 | medium |
| 1472 | medium |
| 1437 | medium |
| 1388 | medium |
| 1333 | medium |
| 1314 | medium |
| 1295 | medium |
| 1246 | medium |
| 1205 | strong |
| 1184 | medium |
| 1147 | medium |
| 1072 | medium |
| 1014 | medium |
| 928 | medium |
| 901 | medium |
| 842 | medium |
| 824 | strong |
| 772 | medium |
| 736 | medium |
| 716 | medium |
| 656 | strong |
| 636 | strong |
| 601 | medium |
| 531 | medium |
| 511 | medium |
| 467 | medium |
| 440 | medium |
| 397 | medium |
| 329 | medium |
| 274 | medium |
| 170 | strong |

Form C melts with an onset temperature of about 149.45° C. and a peak temperature of about 153.50° C. based on DSC analysis. Thermogravimetric analysis shows a 4.3% loss of mass between 40° C. and 160° C. (1 molecule of water=4.2%; leriglitazone·xH₂O, wherein x=1), followed by a loss of mass due to the decomposition starting at 190° C. See FIG. 10.

### Example 4

### Formulation Development Studies and Manufacturing Process of Oral Suspension

Formulation development focused on the evaluation and determination of leriglitazone drug product quality characteristics for the desired dosage design and included viscosity studies, pH studies, optimization of organoleptic characteristics, preservative efficacy studies (challenge tests), homogeneity studies, syringe dosage tests, and dissolution studies. It was surprisingly discovered during these studies that admixing leriglitazone HCl, e.g., Form A, with water resulted in the formation of leriglitazone hydrate Form C.

### Viscosity Studies

Microcrystalline cellulose (FMC RC-591), carboxymethylcellulose sodium (BLANOSE 9H4XF and BLANOSE 9M31F, where BLANOSE 9M31F has a lower viscosity than BLANOSE 9H4XF), and hydroxyethylcellulose (Natrosol® 250HX) were incorporated in different concentrations to create several formulations P7-P11, P16 and P17 of varying viscosity and presented in Table 6.

TABLE 6
Viscosity Studies

| | % w/v (g/100 mL) | | | | | | |
|---|---|---|---|---|---|---|---|
| Ingredients | P7 | P8 | P9 | P10 | P11 | P16 | P17 |
| Leriglitazone HCl | 1.000 | — | 1.000 | 1.000 | — | — | — |
| Sorbitol powder | — | 8.000 | 8.000 | 8.000 | 8.000 | — | — |
| Glycerin | 10.000 | — | — | — | — | 10.000 | 10.000 |
| Colloidal microcrystalline | 2.000 | 2.000 | 2.000 | 1.000 | 1.000 | 1.000 | 0.500 |
| cellulose (FMC RC-591) | | | | | | | |
| Carboxymethylcellulose | — | -— | 1.000 | 0.500 | 0.500 | — | — |
| sodium (BLANOSE 9M31F) | | | | | | | |
| Carboxymethycellulose | — | 1.000 | — | — | — | — | — |
| sodium (BLANOSE 9H4XF) | | | | | | | |
| Hydroxyethylcellulose | 2.000 | — | — | — | — | 1.000 | 0.500 |
| (Natrosol 250 HX) | | | | | | | |
| Saccharin sodium | 0.100 | 0.100 | 0.100 | 0.100 | 0.100 | 0.100 | 0.100 |
| Sodium benzoate | — | — | — | — | 0.100 | 0.100 | 0.100 |
| Sodium acetate | — | — | — | pH | pH | pH | pH |
| trihydrate/Acetic acid | | | | | | | |
| 2N (q.s.) | | | | | | | |
| Water (q.s.) | 100 | 100 | 100 | 100 | 100 | 100 | 100 |
| | mL | mL | mL | mL | mL | mL | mL |
| Viscosity (by visual | Very | High | High | High | Low | High | High but |
| method) | high | | | | | | lower than |
| | | | | | | | P16 |

Abbreviations: q.s., quantity sufficient; w/v, weight/volume; “—”, not tested because they were placebo samples

Pure vehicle compositions and formulations were studied. Formulations P7, P8, and P9 had a high or very high viscosity. Formulations P10, P11, P16, and P17 showed a better flow than formulations P7-P9. The use of acetic acid produced an inconvenient smell and thus formulations with sodium citrate/citric acid were used in further studies. The choice and type of excipients (colloidal microcrystalline cellulose and carboxymethyl cellulose sodium) were also studied in these tests.

### pH Study

Formulations P19-P21, P24 and P26 at different pH values (Table 7) were evaluated to investigate the physicochemical and microbiological stability. The preservative agents congruent with the pH of each formulation were chosen.

The first attempt was at pH value of 5.0 (P19, P20, and P21). This pH was adjusted with sodium citrate and 0.1 M citric acid; methylparaben and propylparaben were added as preservatives. Concurrently, pH 4.0 was investigated (P24 and P26); sodium benzoate was selected as the preservative. One impurity was detected in the suspension at pH=5.0. At pH-4.0, the impurity was absent; thus, development of the formulation with pH=4.0 was continued. To ensure pH stability, a sodium citrate/citric acid buffer was added (P26).

TABLE 7
pH Studies

| | % w/v (g/100 mL) | | | | |
|---|---|---|---|---|---|
| Ingredients | P19 | P20 | P21 | P24 | P26 |
| Leriglitazone HCl | 0.900 | 0.900 | 1.000 | 1.500 | 1.500 |
| Sorbitol powder | 8.000 | — | — | 8.00 | 8.000 |
| Glycerine | — | 10.00 | 10.000 | — | — |
| Colloidal microcrystalline | 1.000 | 0.500 | 0.500 | 1.000 | 1.000 |
| cellulose (FMC RC-591) | | | | | |
| Carboxymethylcellulose sodium | 0.500 | — | — | 0.500 | 0.500 |
| (BLANOSE 9M31F) | | | | | |
| Hydroxyethyl cellulose (Natrosol | 2.000 | 0.500 | 0.500 | — | — |
| 250 HX) | | | | | |
| Methylparaben | 0.135 | 0.135 | 0.135 | — | — |
| Propylparaben | 0.040 | 0.040 | 0.040 | — | — |
| Sodium benzoate | — | — | — | 0.100 | 0.100 |
| Sodium citrate | 0.867 | 0.867 | 0.867 | — | 0.500 |
| Citric acid 0.1M solution (q.s.) | pH 5.0 | pH 5.0 | pH 5.0 | — | — |
| Citric acid 0.2M solution | — | — | — | — | pH 4.0 |
| NaOH solution 0.5N | — | — | — | pH 4.0 | — |
| Saccharin sodium | 0.100 | 0.100 | 0.050 | 0.050 | 0.050 |
| Water (q.s.) | 100 mL | 100 mL | 100 mL | 100 mL | 100 |
| | | | | | mL |
| pH | 5.0 | 5.0 | 5.0 | 4.0 | 4.0 |

Abbreviations: q.s., quantity sufficient; w/v, weight/volume; “—”, not tested because they were placebo samples

### Optimization of Organoleptic Properties

Organoleptic properties (sensory and viscosity) of formulation P26 improved by the addition of strawberry flavour (formulation P32). The standard strawberry flavour concentration recommended by the manufacturer was selected (0.015 g/100 mL). The formulation is presented in Table 8.

TABLE 8
Formulation with Optimized Organoleptic Characteristics

| Formulation P32 | % w/v (g/100 mL) |
|---|---|
| Leriglitazone HCl (sieved at 125 μm) | 1.500 |
| Sorbitol | 8.000 |
| Colloidal microcrystalline cellulose (FMC RC-591) | 1.000 |
| Carboxymethylcellulose sodium (BLANOSE 9M31F) | 0.500 |
| Saccharin sodium | 0.050 |
| Sodium benzoate | 0.100 |
| Sodium citrate | 0.500 |
| Citric acid monohydrate 0.2M solution (q.s.) | pH 4.0 |
| Strawberry flavour | 0.015 |
| Water (q.s.) | 100 mL |

The resulting suspension (P32) exhibited a homogeneous white appearance, with absence of particle agglomerates (occasional tiny particles seen on the wall of its glass container), and correct flowability after gentle stirring. The viscosity of this suspension was tested with a Brookfield viscometer and the results are presented in Table 9. The positive outcome of this formulation (P32) made to progress the development with it.

TABLE 9
Viscosity of Suspension P32

| | | | | | Viscosity (cp) |
|---|---|---|---|---|---|
| | % | | | | After rest + |
| | Colloidal | % CMC | Viscosity | Viscosity | stirring 5 |
| | MCC | Na | (cp) | (cp) | minutes |
| Batch | (RC-591) | (9M31F) | a T₀ | after rest | at 540 rpm |
| P32 | 1.000 | 0.500 | 4,900 | 4,871 | 3,306 |
| | | | (LV2 at | (LV2 at | (LV2 at 5 rpm) |
| | | | 5 rpm) | 5 rpm) | |

A centipoise (cp) is one millipascal second (mPa*s) in SI units.

The amount of preservative for formulation P32 was evaluated. A set of formulas inspired by suspension P32 and prepared at bench scale, with different preservative (sodium benzoate) concentrations were manufactured under engineering conditions to preliminary evaluate effectiveness. The formulas and results are presented in Table 10.

TABLE 10
Challenge Test Formulas and Summary Results

| Batch | Preservative content | TAMC | TYMC |
|---|---|---|---|
| P39 | leriglitazone 13.66 mg/mL | 30 CFU/g | <10 CFU/g |
| | Preservative content 1.0 mg/ml | | |
| P40 | leriglitazone 13.66 mg/mL | <10 CFU/g | <10 CFU/g |
| | Preservative content 1.2 mg/ml | | |
| P45 | leriglitazone 13.66 mg/mL | <10 CFU/g | <10 CFU/g |
| | Preservative content 1.5 mg/mL | | |

Abbreviations: CFU, colony-forming unit; TAMC, total aerobic microbial count; TYMC, total yeast and mold count.

All three batches fulfilled the total aerobic microbial count (TAMC) and total yeast and mold count (TYMC) requirements of the European Pharmacopoeia (Ph. Eur.) and United States Pharmacopeia (USP) for this type of formulation, <100 CFU/mL TAMC and <10 CFU/mL TYMC.

The efficacy of the antimicrobial preservation in the leriglitazone 13.66 mg/ml oral suspension was then investigated in one GMP pilot batch (scale of 22 L). This batch was coded 160001 and included 1.0 mg/mL preservative (sodium benzoate). The results of the study are shown in the Table 11

TABLE 11
Efficacy of Antimicrobial Preservation leriglitazone 13.66 mg/mL
(preservative content 1.0 mg/mL, 0.1% w/v) Batch 160001

| | T = | T = | T = | |
|---|---|---|---|---|
| Test strain | 0 hours | 14 days | 28 days | Conclusions |
| Candida albicans | 6.8 × 10⁵ | <10 | <10 | Complies |
| ATCC 10231 | | | | |
| Aspergillus brasiliensis | 6.0 × 10⁵ | <10 | <10 | Complies |
| ATCC 16404 | | | | |
| Pseudomonas | 1.4 × 10⁶ | <10 | <10 | Complies |
| aeruginosa ATCC 9027 | | | | |
| Staphylococcus aureus | 5.3 × 10⁵ | <10 | <10 | Complies |
| ATCC 6538 | | | | |
| Escherichia coli ATCC | 1.4 × 10⁶ | <10 | <10 | Complies |
| 8739 | | | | |

Results expressed in CFU/g of product.
Abbreviations: ATCC, American Type Culture Collection; CFU, colony-forming unit

The plate-count method and the diluent used in the efficacy of antimicrobial preservation test were effective in the recovery of the growth of microorganisms in the proposed formulation. Batch 160001 complied with the criteria of efficacy of antimicrobial preservation for oral preparations according to the USP and Ph. Eur. The lower concentration of preservative (1.0 mg/mL sodium benzoate) in batch 160001 demonstrated effectiveness and it was the preservative concentration chosen for leriglitazone 13.66 mg/mL oral suspension.

Leriglitazone 13.66 mg/mL oral suspension batch 160001 (sodium benzoate 0.1% w/v) was placed on an ICH long-term stability program. Microbial testing is performed to evaluate the effectiveness of sodium benzoate over time. The challenge test will be repeated at the end of the stability study with samples stored at long-term condition.

A batch with a higher concentration of preservative 1.5 mg/mL (0.15% w/v), “leriglitazone 13.66 mg/ml oral suspension 100 mL CON,” batch 170001 CON (scale 22 L), was studied to bracket the preservative effectiveness of similar formula and target concentration of preservative (leriglitazone 13.66 mg/ml oral suspension 100 mL, batch 160001), in case there was any growth in microbial concentration during its stability tests. It also showed effective microbial preservation.

The same batch was studied after a period of 36 months, and it has been demonstrated compliance. The results are presented in Table 12.

TABLE 12
Efficacy of Antimicrobial Preservation on an
Overpassed Shelf-life Batch

| | T = | T = | T = | |
|---|---|---|---|---|
| Test strain | 0 hours | 14 days | 28 days | Conclusions |
| Candida albicans | 2.0 × 10⁶ | <10 | <10 | Complies |
| ATCC 10231 | | | | |
| Aspergillus | 1.2 × 10⁶ | <10 | <10 | Complies |
| brasiliensis ATCC | | | | |
| 16404 | | | | |
| Pseudomonas | 2.6 × 10⁶ | <10 | <10 | Complies |
| aeruginosa ATCC | | | | |
| 9027 | | | | |
| Staphylococcus aureus | 2.1 × 10⁶ | <10 | <10 | Complies |
| ATCC 6538 | | | | |
| Escherichia coli | 1.4 × 10⁶ | <10 | <10 | Complies |
| ATCC 8739 | | | | |

Results expressed in CFU/g of product.
Abbreviations: ATCC, American Type Culture Collection; CFU, colony-forming unit

### Homogeneity Study

The suspension homogeneity is a critical attribute due to the characteristics of the leriglitazone drug substance and its concentration in the formula. Poor homogeneity of the suspension could negatively impact the reliability of the administered dose.

The homogeneity of the chosen formulation was investigated in samples of a representative batch of leriglitazone 13.66 mg/mL oral suspension (batch 160001) after shaking the bottles 30 seconds by hand, which is the recommended handling before extraction of the clinical doses. Ten bottles were analysed in duplicate. These bottles were taken randomly at time intervals during the filling in bottles operation of this batch. The results are shown in Table 13. All data obtained are within the range of 100±5% for leriglitazone assay and 100±10% for sodium benzoate assays.

TABLE 13
Leriglitazone 13.66 mg/mL Oral Suspension (Batch 160001)
Homogeneity Test

| | Leriglitazone Assay | Sodium Benzoate Assay |
|---|---|---|
| Sample | Results (%) | Results (%) |
| 1-1 | 98.0 | 102.7 |
| 1-2 | 97.8 | 102.5 |
| 2-1 | 99.5 | 102.6 |
| 2-2 | 99.5 | 101.1 |
| 3-1 | 99.6 | 102.0 |
| 3-2 | 99.5 | 102.1 |
| 4-1 | 98.2 | 100.5 |
| 4-2 | 97.8 | 99.6 |
| 5-1 | 100.3 | 103.3 |
| 5-2 | 100.5 | 102.7 |
| 6-1 | 100.0 | 103.4 |
| 6-2 | 99.8 | 102.0 |
| 7-1 | 100.3 | 103.4 |
| 7-2 | 100.3 | 102.8 |
| 8-1 | 100.8 | 103.4 |
| 8-2 | 101.2 | 104.8 |
| 9-1 | 99.7 | 102.3 |
| 9-2 | 99.9 | 103.9 |
| 10-1 | 101.6 | 104.2 |
| 10-2 | 101.6 | 104.0 |
| Mean | 99.8 | 102.7 |
| Standard | 1.152 | 1.266 |
| Deviation | | |
| Coefficient of | 1.15 | 1.23 |
| Variation | | |

The results indicate that this bulk test formulation (13.66 mg/mL leriglitazone, 1.0 mg/mL sodium benzoate) has an adequate homogeneity as tested in the clinical style dosing bottles.

The results endorse the homogeneity of leriglitazone oral suspension within the multi-dose container once at rest. They are fully coherent with the thixotropic nature of this formulation. Leriglitazone content determined in upright and inverted samples of same batch and ageing, and after 30 s of shaking were of 101.4% and 99.8%, respectively.

In addition, the homogeneity was checked at every batch bulk, testing sampling samples which are taken at different positions of the manufacturing process.

It is considered that there is no need to introduce it in stability since the attribute of absence of sediment would indicate inhomogeneity if sediments were observed. The homogeneity is always assured before the administration with the 30 s of shaking and this will be included in the leaflet accordingly.

### Syringe Dosage Test

A syringe dosage study was performed to determine the accuracy of dosing leriglitazone oral suspension within the range of 0.5-10 mL with a 10 ml syringe. For the study ten syringes (n=10) were loaded with the nominal volume chosen and unloaded in a single shot. The dispense test is an in-house method reproducing faithfully the manipulation done by the patients.

The dispensed amount of leriglitazone oral suspension was directly weighed, and the result transformed in volume, taking into account the density of the specific leriglitazone oral suspension batch used in this study (GMP batch 170001 ESC used in Phase ⅔ clinical studies; density 1.04 g/mL). This provided a mean range as a measure of in-use accuracy and precision. The results of the study, presented in Table 14, indicate a mean syringe dose delivery range (precision/accuracy) of ±0.03 mL at 0.5% RSD under the condition tested. This is within the manufacturer's tolerances of ±0.59 mL for the 10 mL syringe.

TABLE 14
Syringe Dosing Tests (10 ml Syringe)
Volume Dispensed (mL)

| Nominal | Mean | | | | | Diff. | Diff. |
|---|---|---|---|---|---|---|---|
| Volume | (n = | | RSD | | | Max- | Nom- |
| (mL) | 10) | SD | (%) | Min | Max | Min | Mean |
| 0.5 | 0.49 | 0.01 | 1.2 | 0.48 | 0.50 | 0.02 | 0.01 |
| 1.0 | 0.99 | 0.00 | 0.5 | 0.99 | 1.00 | 0.01 | 0.01 |
| 1.5 | 1.49 | 0.02 | 1.2 | 1.46 | 1.53 | 0.06 | 0.01 |
| 2.0 | 1.99 | 0.02 | 0.8 | 1.96 | 2.01 | 0.05 | 0.01 |
| 2.5 | 2.48 | 0.02 | 0.6 | 2.46 | 2.50 | 0.04 | 0.02 |
| 3.0 | 2.99 | 0.01 | 0.4 | 2.97 | 3.00 | 0.03 | 0.01 |
| 3.5 | 3.46 | 0.02 | 0.5 | 3.44 | 3.48 | 0.05 | 0.04 |
| 4.0 | 3.95 | 0.02 | 0.4 | 3.93 | 3.98 | 0.05 | 0.05 |
| 4.5 | 4.46 | 0.02 | 0.4 | 4.43 | 4.48 | 0.05 | 0.04 |
| 5.0 | 4.95 | 0.02 | 0.4 | 4.92 | 4.98 | 0.06 | 0.05 |
| 5.5 | 5.44 | 0.02 | 0.4 | 5.41 | 5.48 | 0.08 | 0.06 |
| 6.0 | 5.93 | 0.03 | 0.4 | 5.88 | 5.97 | 0.08 | 0.07 |
| 6.5 | 6.44 | 0.02 | 0.3 | 6.41 | 6.47 | 0.06 | 0.06 |
| 7.0 | 6.92 | 0.03 | 0.5 | 6.86 | 6.99 | 0.13 | 0.08 |
| 7.5 | 7.43 | 0.02 | 0.3 | 7.40 | 7.46 | 0.06 | 0.02 |
| 8.0 | 7.92 | 0.02 | 0.2 | 7.89 | 7.94 | 0.05 | 0.08 |
| 8.5 | 8.41 | 0.03 | 0.3 | 8.37 | 8.45 | 0.07 | 0.09 |
| 9.0 | 8.90 | 0.03 | 0.4 | 8.65 | 8.95 | 0.09 | 0.1 |
| 9.5 | 9.40 | 0.04 | 0.4 | 9.33 | 9.44 | 0.11 | 0.1 |
| 10.0 | 9.87 | 0.03 | 0.3 | 9.84 | 9.91 | 0.07 | 0.13 |
| Mean | | 0.02 | 0.50 | | | 0.06 | 0.05 |
| Range | | | | | | ±0.03 | |

Abbreviations: Diff Max-Min, difference between maximum and minimum values; Diff Nom-Mean, difference between nominal and mean values; Min, minimum value; Max, maximum value; SD, standard deviation; RSD, relative standard deviation as percentage; SD, standard deviation

The selected 10 mL syringe was shown to be suitable for administering leriglitazone 13.66 mg/mL oral suspension along the complete dosing range of 0.5 to 10 mL.

### Other Properties of the Oral Suspension

Dissolved fraction: Based upon the intrinsic physicochemical properties of leriglitazone HCl, the fraction dissolved at the oral suspension pH should be negligible. The formula contains a viscosifying matrix that provides elasticity to the suspension, which in turn interferes with a reliable direct determination of leriglitazone in the aqueous phase of the oral suspension. Therefore, a dummy determination was performed during formulation development to simulate the worst-case scenario. Dissolved leriglitazone was measured in a composition mimicking leriglitazone 13.66 mg/mL oral suspension but in absence of the viscosifying system (i.e., aqueous solution), and thus making centrifugation possible. Even in these conditions, the dissolved leriglitazone did not exceed 0.5% of the total dose.

Particle size distribution/dissolution: The effect of the DS particle size on the dissolution rate of leriglitazone oral suspension was evaluated as part of the dissolution method development. Based on the results, it was determined that the dissolution comply with the specification acceptance criteria with DS PSD of D90=132 μm. Furthermore, the leriglitazone oral suspension release and stability results at both accelerated and long-term conditions comply with the proposed specifications of not less than 80% (Q) at 30 min.

### Manufacturing Process

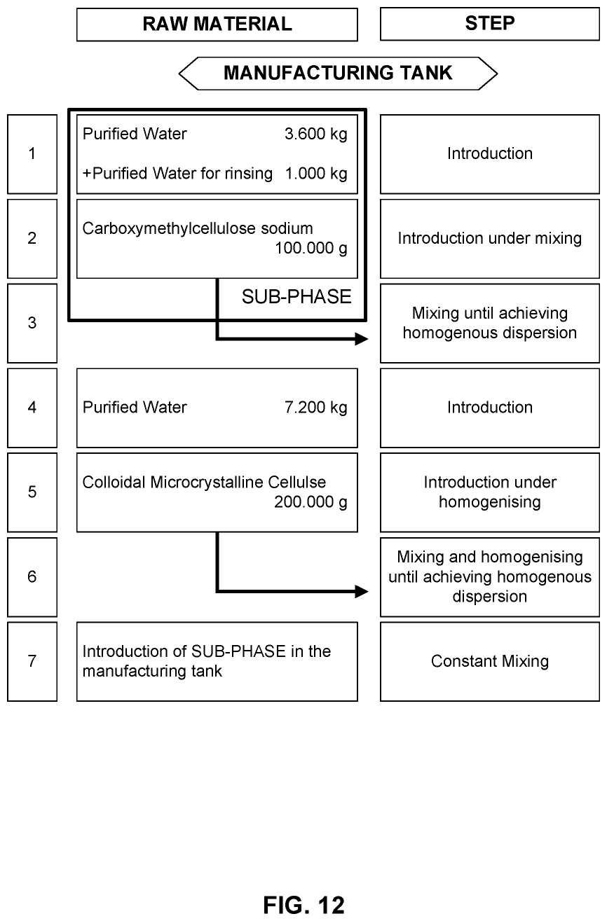

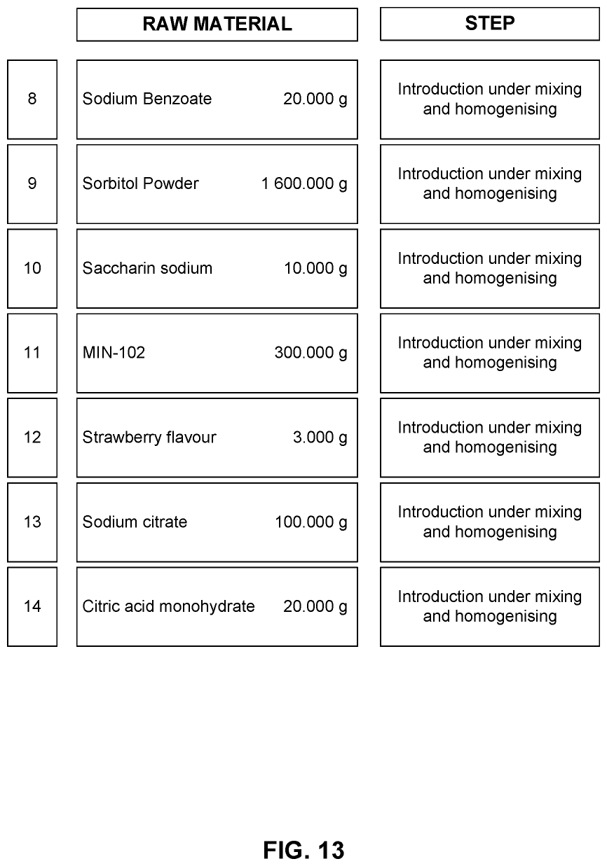

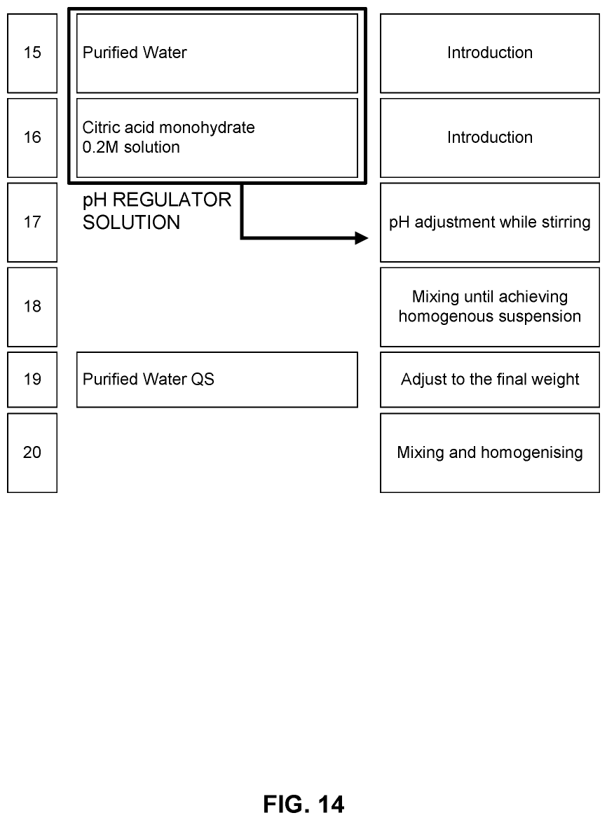

The pharmaceutical formulations described herein may be prepared according to the manufacturing process flow chart provided in FIGS. 12-14.

### Example 5

TABLE 15
Impurity Analysis of Leriglitazone Oral Suspension at
pH 4.0 and 5.5

| | | pH | |
|---|---|---|---|
| Controls | | 4.0 | 5.5 |
| Impurities | Impurity A | <0.05% | <0.05% |
| (HPLC)* | Impurity B | <0.05% | <0.05% |
| | Impurity C | <0.05% | <0.05% |
| | Impurity D | <0.05% | <0.05% |
| | Impurity E | <0.05% | <0.05% |
| | Impurity F | <0.05% | <0.05% |
| | Impurity G | <0.05% | <0.05% |
| | Unknown Impurity | <0.05% | 0.16% |
| | Impurity H | ND | ND |
| | (by UPLC) | | |
| | Total impurities | <0.05% | 0.16% |

| | | | | | | | | |
|---|---|---|---|---|---|---|---|---|
| Ratio of leriglitazone isomers | 1 | 2 | 3 | 4 | 1 | 2 | 3 | 4 |
| | 23.6 | 26.3 | 26.3 | 23.9 | 24.0 | 26.4 | 26.1 | 23.6 |

*LOQ: 0.05, ND: not detected

TABLE 16
Chromatographic Conditions

| Parameter | Instrumental Settings |
|---|---|
| Instrument | Agilent (or equivalent) HPLC quaternary with |
| | DAD detector |
| Column | Agilent Eclipse Plus (or equivalent) C18, 150 × |
| | 2.1 mm; 3.5 μm |
| Flow rate | 0.6 mL/min |
| Injection volume | 5.0 μL |
| Detector/Wavelength | UV, 225 nm and 270 nm (VWD)/DAD: 190-450 |
| | nm (for peak purity) |
| Column temperature | 40° C. |
| Run time | 50 min |

TABLE 17
Gradient Program

| Time (min) | Mobile Phase A % | Mobile Phase B % |
|---|---|---|
| 0.0 | 90 | 10 |
| 8.0 | 88 | 12 |
| 18.0 | 88 | 12 |
| 23.0 | 65 | 35 |
| 25.0 | 65 | 35 |
| 40.0 | 5 | 95 |
| 45.0 | 5 | 95 |
| 45.1 | 90 | 10 |
| 50.0 | 90 | 10 |

TABLE 18
HPLC Mobile Phases and Solutions

| | |
|---|---|
| Mobile phase A | 10 mM NaH₂PO₄ pH 2.80 in purified water |
| Mobile phase B | ACN |
| Dilution solution | Water: EtOH, 50:50 v/v |

TABLE 19
Chemical Structures of Impurities

| Specified | | |
|---|---|---|
| Impurity | Chemical Structure | Chemical Name |
| Impurity B | 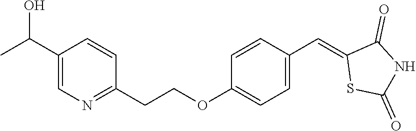 [SMILES: CC(O)c1ccc(CCOc2ccc(/C=C3\SC(=O)NC3=O)cc2)nc1] | (Z)-5-(4-(2-(5-(1- hydroxyethyl)pyridin-2- yl)ethoxy)benzylidene)thiazolidine- 2,4-dione |
| Impurity C | 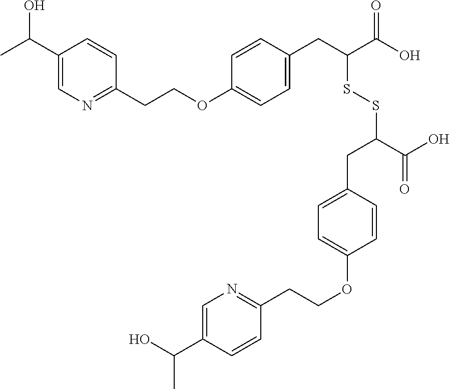 [SMILES: CC(O)c1ccc(CCOc2ccc(CC(SSC(Cc3ccc(OCCc4ccc(C(C)O)cn4)cc3)C(=O)O)C(=O)O)cc2)nc1] | 2,2′-disulfanediylbis(3-(4-(2-(5-(1- hydroxyethyl)pyridin-2- yl)ethoxy)phenyl)propanoic acid) |
| Impurity D | 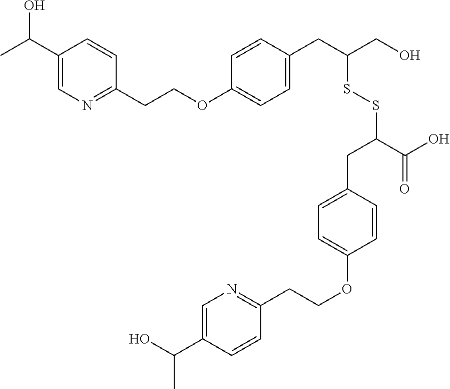 [SMILES: CC(O)c1ccc(CCOc2ccc(CC(CO)SSC(Cc3ccc(OCCc4ccc(C(C)O)cn4)cc3)C(=O)O)cc2)nc1] | 2-((1-hydroxy-3-(4-(2-(5-(1- hydroxyethyl)pyridin-2- yl)ethoxy)phenyl)propan-2- yl)disulfaneyl)-3-(4-(2-(5-(1- hydroxyethyl)pyridin-2- yl)ethoxy)phenyl)propanoic acid |
| Impurity H | 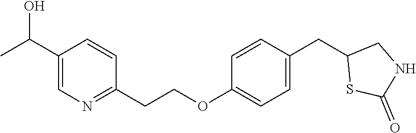 [SMILES: CC(O)c1ccc(CCOc2ccc(CC3CNC(=O)S3)cc2)nc1] | 5-(4-(2-(5-(1- hydroxyethyl)pyridin-2- yl)ethoxy)benzyl)thiazolidin-2-one |

### Synthesis of Impurity B

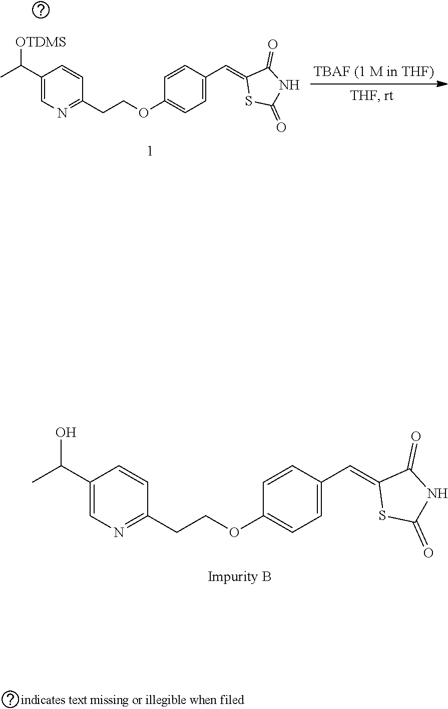

Compound 1, see WO 2018/116281, was dissolved in THF and a TBAF solution 1M in THF was added. The mixture was stirred overnight at room temperature (rt) and concentrated under vacuum. Then the crude solid obtained was dissolved in DCM and the reaction mixture was filtrated on a silica plug (AcOEt/Tol 8/2). The filtrate was concentrated under vacuum to give Impurity B as a yellow oil. The structure of Impurity B was confirmed by ¹NMR and HPLC-MS.

### Synthesis of Impurity C

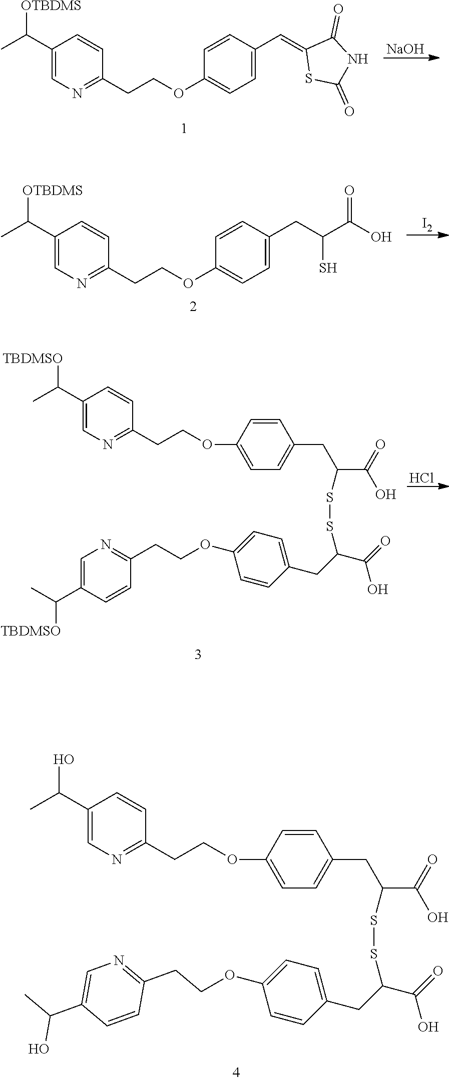

Compound 1 was dissolved in acetonitrile at 50° C. under nitrogen atmosphere. Aqueous NaOH solution (30%) was added and the solution was stirred at 50° C. during 5 h. The reaction mixture was quenched by adding an aqueous solution of KHSO₄ and the organic phase was diluted with diethyl ether, separated, and dried to obtain a solution of Compound 2.

Iodine was added portion-wise to the solution of Compound 2 under nitrogen atmosphere and the solution was stirred overnight at rt. The reaction mixture was quenched with thiosulfate solution and the phases were separated, dried, and concentrated to afford crude Compound 3.

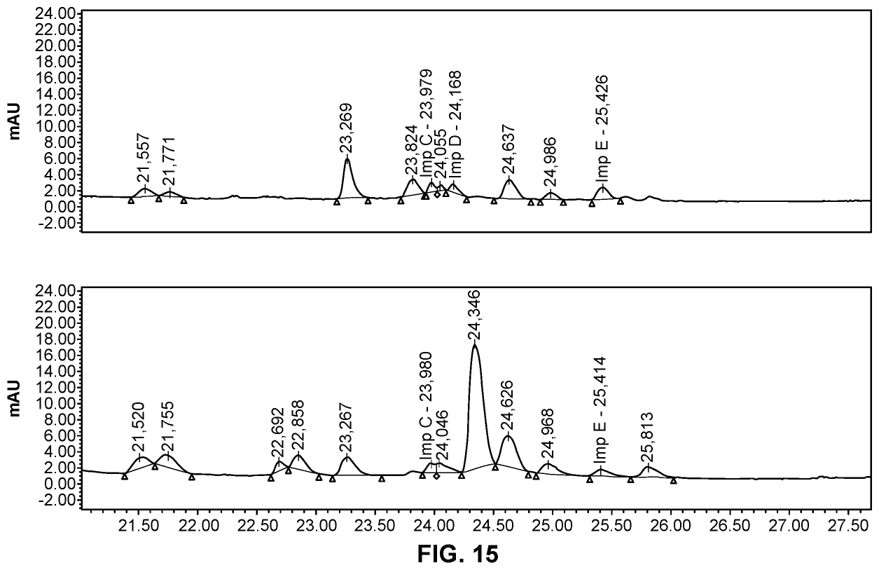

The crude Compound 3 was dissolved in THF and deprotected using HCl to generate Compound 4 (Impurity C), which was further purified by reverse phase chromatography. The structure of Impurity C was confirmed by ¹NMR and HPLC-MS. See FIG. 15.

### Synthesis of Impurity D

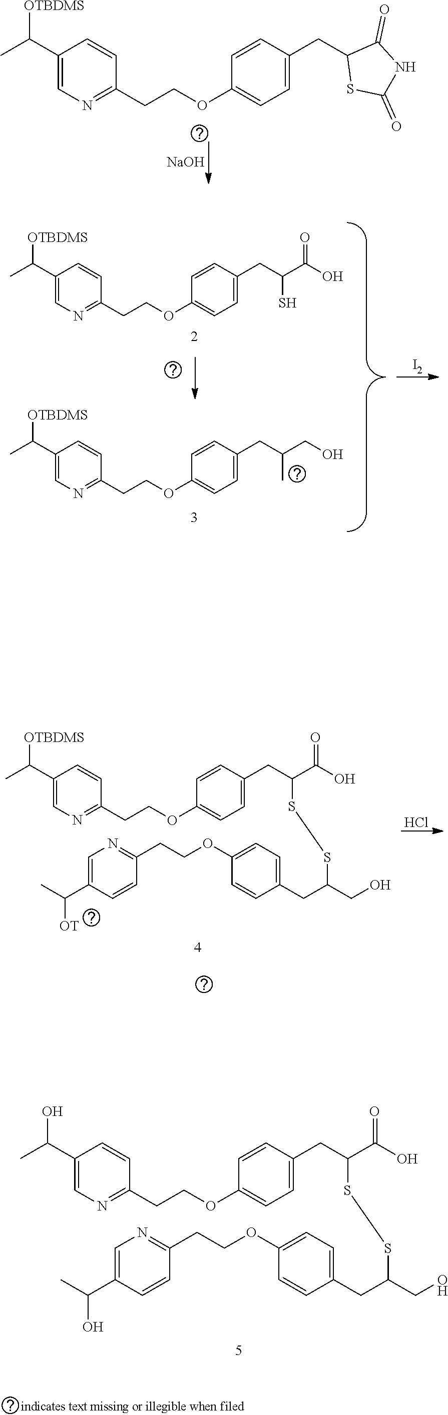

Compound 1 was dissolved in acetonitrile at 50° C. under nitrogen atmosphere. Aqueous NaOH solution (30%) was added and the solution was stirred at 50° C. during 4 h. The reaction mixture was quenched by adding an aqueous solution of KHSO₄ and the organic phase was diluted with iPrOAc, separated, dried, and concentrated to obtain crude Compound 2.

Half of the crude Compound 2 was dissolved in THE, cooled to 2-5° C., and a solution of borane THF complex was added dropwise maintaining the temperature. The solution was then stirred at 5° C. for 2 h and at rt for 2 h. Methanol was slowly added to quench the reaction and the mixture was stirred over the weekend at rt. The mixture was concentrated to afford Compound 3 as a crude.

To a solution of Compound 2 in THF was added Compound 3 in THF and iodine portion-wise. The mixture is stirred overnight at rt. The reaction was quenched by adding an aqueous solution of thiosulfate, then water and EtOAc were added and the phases were separated. The organic phases were dried and concentrated to afford Compound 4 as a crude.

The crude mixture of Compound 4 was dissolved in THF and aqueous HCl was added slowly. The solution was stirred at rt for 3 h. Saturated aqueous bicarbonate solution as added and the mixture was concentrated in vacuo.

The residue is taken up in methanol and filtered, and the crude product was purified via reverse phase chromatography. A second purification via preparative chromatography (SFC) was done to obtain the pure compound 5 (Impurity D). The structure of Impurity D was confirmed by ¹NMR and HPLC-MS.

### Synthesis of Impurity H

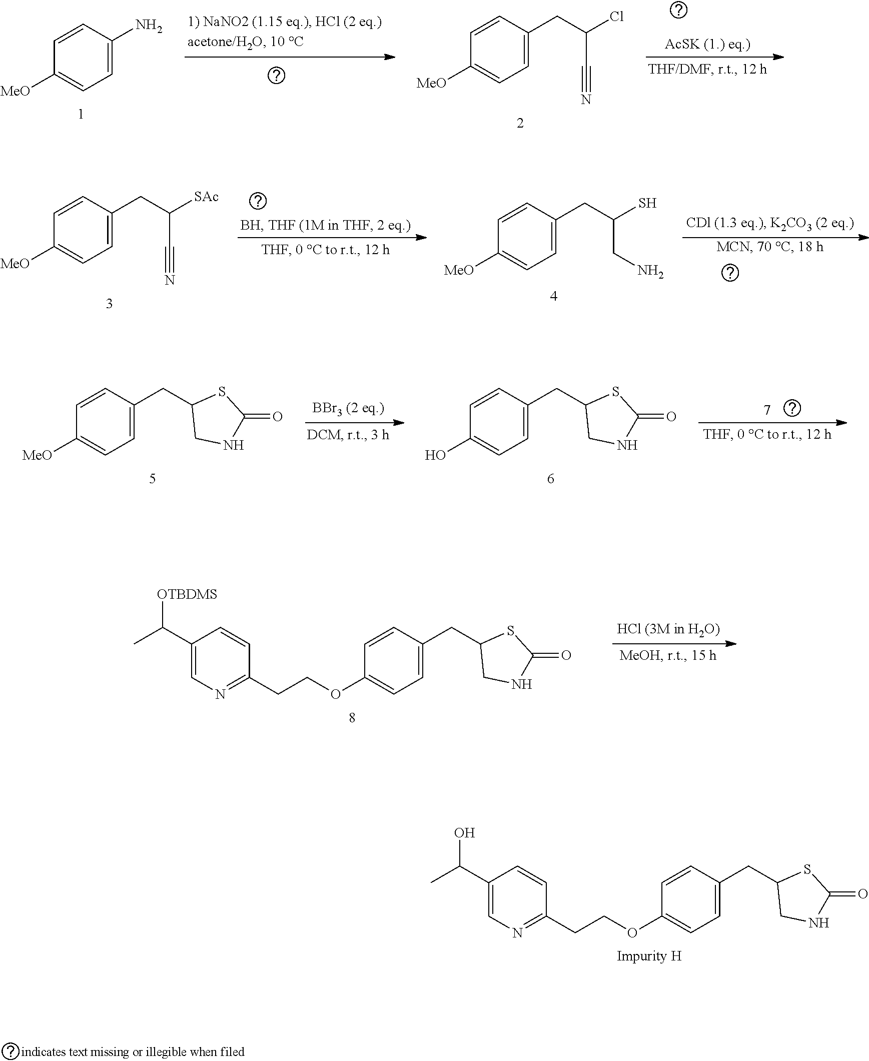

Compound 1 was first treated with NaNO₂ and HCl in a mixture of acetone and water at 10° C. and then AcONa, acrylonitrile, and CuCl_{2.2}H₂O was added and the reaction mixture was heated to 40° C. for 4 h to obtain Compound 2.

Compound 2 was treated with AcSK in a mixture of THF/DMF at rt during 12 h to afford Compound 3.

BH₃. THF was added to a solution of Compound 3 in THF at 0° C. and the reaction was warmed to rt for 12 h to obtain Compound 4.

Compound 4 was coupled with CDI in acetonitrile using K₂CO₃ as the base. The reaction was heated to 70° C. for 18 h to afford Compound 5.

Compound 5 was demethylated using BBr₃ in DCM at rt for 3 h to afford Compound 6.

A Mitsunobu reaction between Compound 6 and Compound 7 (IRP2-1, see WO 2018/116281) using DIAD and PPh₃ in THF from 0° C. to rt over 12 h afforded Compound 8.

Deprotection of Compound 8 under acidic conditions (using HCl) afforded Impurity H.

The structure of Impurity H was confirmed by ¹NMR and HPLC-MS.

Having now fully described this disclosure, it will be understood by those of ordinary skill in the art that the same can be performed within a wide and equivalent range of conditions, formulations, and other parameters without affecting the scope of the invention or any embodiment thereof.

Other embodiments of the disclosure will be apparent to those skilled in the art from consideration of the specification and practice of the invention disclosed herein. It is intended that the specification and examples be considered exemplary only, with a true scope and spirit of the invention being indicated by the following claims.

All patents, patent applications, and publications cited herein are fully incorporated by reference herein in their entirety.

1. Crystalline leriglitazone hydrate Form C characterized as having:
(i) a x-ray powder diffraction (XRPD) pattern with peaks at 12.7, 15.4, 19.1, 21.0, 23.8, and 25.9 degrees 2Θ using Cu Kα radiation, wherein the 2Θ values are ±0.2 degrees 2Θ; or
(ii) a x-ray powder diffraction pattern with d-spacings at 6.99, 5.74, 4.65, 4.23, 3.73, and 3.44 Å using Cu Kα radiation; or
(iii) a FT-Raman spectrum with peaks at 3056, 2960, 2992, 1735, 1608, 1205, 824, 656, and 170 cm⁻¹, wherein the cm⁻¹ values are ±4 cm⁻¹; or
(iv) a melting point with an onset temperature of about 149.45° C. and a peak temperature of about 153.50° C. based on differential scanning calorimetry; or
a combination (i), (ii), (iii), and/or (iv).

2-14. (canceled)

15. The crystalline leriglitazone hydrate Form C of claim 1, characterized as having about 4.3% loss of mass between 40° C. and 160° C. based on thermal gravimetric analysis.

16. (canceled)

17. A pharmaceutical composition comprising a suspension of the crystalline leriglitazone hydrate Form C of claim 1 in water, and one or more pharmaceutically acceptable carriers, diluents or excipients.

18. Crystalline leriglitazone Form B characterized as having:
(i) a x-ray powder diffraction (XRPD) pattern with peaks at 16.6, 18.8, 21.1, 21.8, 23.8, and 24.7 degrees 2Θ using Cu Kα radiation, wherein the 2Θ values are ±0.2 degrees 2ϑ; or
(ii) a x-ray powder diffraction pattern with d-spacings at 5.33, 4.73, 4.21, 4.07, 3.73, and 3.60 Å using Cu Kα radiation; or
(iii) a melting point with an onset temperature of about 149.34° C. and a peak temperature of about 153.56° C. based on differential scanning calorimetry; or
a combination (i), (ii), and/or (iii).

19-32. (canceled)

33. A pharmaceutical composition comprising a suspension of the crystalline leriglitazone Form B of claim 18 in water, and one or more pharmaceutically acceptable carriers, diluents or excipients.

34. A pharmaceutical formulation comprising:
(a) the crystalline leriglitazone hydrate Form C of claim 1, in an amount from 0.01% w/v to 5% w/v;
(b) one or more cellulose derivatives in an amount from 0.01% w/v to 2% w/v, wherein the one or more cellulose derivatives have a viscosity between 1500 and 4500 mPa*s, preferably between 1500 to 3500 mPa*s, more preferably between 1500 and 3000 mPa*s as measured by Brookfield viscosity method; and
(c) water QS to 100%.

35-38. (canceled)

39. The pharmaceutical formulation according to claim 34, wherein the leriglitazone hydrate Form C is present in an amount from 1% w/v to 2% w/v.

40. The pharmaceutical formulation according to claim 34, wherein the one or more cellulose derivatives comprise microcrystalline cellulose, carboxymethylcellulose sodium, or mixtures thereof.

41-51. (canceled)

52. The pharmaceutical formulation of claim 34 comprising:
(a) crystalline leriglitazone hydrate Form C; in an amount of 1.0% to 2.0% w/v;
(b) about 1% w/v of microcrystalline cellulose;
(c) about 0.5% w/v of carboxymethyl cellulose sodium;
(d) water QS to 100%;
and, optionally;
(e) about 8% w/v sorbitol powder;
(f) about 0.05% w/v saccharin sodium;
(g) about 0.1% w/v sodium benzoate;
(h) about 0.5% w/v sodium citrate;
(i) about 0.1% w/v citric acid monohydrate;
(j) about 0.015% w/v strawberry flavour;
(k) about 1% w/v 0.2 M aqueous citric acid monohydrate solution.

53. The pharmaceutical formulation of claim 52 prepared by admixing:
(a) about 1.5% w/v leriglitazone hydrochloride, preferably Form A;
(b) about 8% w/v sorbitol powder;
(c) about 1% w/v microcrystalline cellulose;
(d) about 0.5% w/v carboxymethyl cellulose sodium;
(e) about 0.05% w/v saccharin sodium;
(f) about 0.1% w/v sodium benzoate;
(g) about 0.5% w/v sodium citrate;
(h) about 0.1% w/v citric acid monohydrate;
(i) about 0.015% w/v strawberry flavour;
(j) about 1% w/v 0.2 M aqueous citric acid monohydrate solution; and
(k) water QS to 100%.

54. The crystalline leriglitazone hydrate Form C of claim 1 for use in treating a disease or disorder in a patient in need thereof, wherein the disease or disorder is a central nervous system disease or disorder, mitochondrial disease, a liver disease or disorder, chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, an inflammatory respiratory disease, or a lung disease or disorder.

55. (canceled)

56. The crystalline leriglitazone hydrate Form C claim 54, wherein the central nervous system disease or disorder is selected from the group consisting of neurodegenerative disease, cerebrovascular disease, seizure, epilepsy, viral disease, neuroinflammatory disease, brain tumor, organic acidemias, fatty acid disorder, and genetic mitochondrial disorder, wherein the central nervous system disease or disorder is a rare metabolic disease selected from the group consisting of organic acidemias, fatty acid disorders and genetic mitochondrial disorders, wherein
the mitochondrial disease is a primary mitochondrial disorder selected from the group consisting of Rett syndrome, Alper's disease; Leber's hereditary optic neuropathy (LHON); Kearns-Sayre syndrome (KSS); Leigh's syndrome; Leigh-like syndrome; maternally inherited Leigh syndrome (MILS); mitochondrial depletion syndrome (MDS); mitochondrial DNA depletion syndrome (MDDS); mitochondrial encephalomyopathy; mitochondrial encephalomyopathy with lactic acidosis and stroke-like episodes (MELAS); myoclonic epilepsy with ragged red fibers (MERRF); mitochondrial 1 neurogastrointestinal encephalopathy syndrome (MNGIE); neuropathy, ataxia, and retinitis pigmentosa (NARP); Pearson syndrome; chronic progressive external opthalmoplegia (CPEO); dominant optic atrophy (DOA); autosomal dominant optic atrophy (ADOA); mitochondrial myopathy; cardiomyopathy; mitochondrial encephalopathy; myoclonic epilepsy; maternally inherited diabetes and deafness (MIDD); ataxia neuropathy spectrum; 3-methylglutaconic aciduria; sensoneural deafness; neuroradiological findings of Leigh-like syndrome (MEGDEL); SURF1 (COX definient Leigh syndrome due to complex IV surfeit protein deficiency); oxidative phosphorylation disorders; Berth syndrome; lethal infantile cardiomyopathy (LIC); pyruvate carboxylase deficiency; pyruvate dehydrogenase deficiency; POLG mutation; isolated or combined OXPHOS deficiencies with so far unsolved genetic defect including disturbed pyruvate oxidation and ATP plus PCr production rates; POLG2 mutation; carnitine-acyl-cartinine deficiency; carnitine deficiency; creatinine deficiency syndromes; Co-Enzyme Q10 deficiency; Complex I deficiency; Complex II deficiency; Complex III deficiency; Complex IV deficiency; Complex V deficiency; lactic acidosis; leukoencephalopathy with brain stem and spinal cord involvement and lactate elevation (LBSL); Luft disease; carnitine palmitoyltransferase (CPT I or CPT II) deficiency; short-chain acyl-CoA dehydrogenase deficiency (SCAD); short-chain 3-hydroxyacetyl-CoA dehydrogenase deficiency (SCHAD); medium-chain acyl-CoA dehydrogenase deficiency (MCAD); multiple acyl-CoA dehydrogenase deficiency (MADD); long-chain acyl-CoA dehydrogenase deficiency (LCAD); very long-chain acyl-CoA dehydrogenase deficiency (VLCAD); trifunctional protein (TFP) deficiency; and glutaric aciduria Type II, wherein
the mitochondrial disease is a secondary mitochondrial disorder selected from the group consisting of Duchenne muscular dystrophy (DMD); Becker muscular dystrophy (BMD); myotonic dystrophy (BMD); congenital myopathies; glycogen storage disorders; spinal-bulbar muscular atrophy (SBMA); argininosuccinic aciduria; autism spectrum disorder (ASD); autoimmune diseases of the skin (such as pemphigus vulgaris and lupus); methylmalonic and propionic acidurias; disorders or purine and/or pyrimidine synthesis; facioscapulohumeral muscular dystrophy (FSHD); congenital muscular dystrophies; collagen VI muscular dystrophies (e.g., Ullrich congenital muscular dystrophy, Bethlem myopathy, oculopharyngeal distal and Emery-Dreifuss); DiGeorge syndrome; and neuromuscular disorders (such as limb-girdle muscular dystrophy, inflammatory myopathies, Charcot Marie Tooth (CMT) neuropathy, and drug-induced peripheral neuropathies), wherein
the lung disease or disorder is an inflammatory lung condition or disease caused by a viral infection, acute inflammation of the lung, or interstitial lung disease, or wherein the liver disease or disorder is nonalcoholic steatohepatitis (NASH) or nonalcoholic fatty liver disease (NAFLD).

57. (canceled)

58. The crystalline leriglitazone hydrate Form C claim 54, wherein the neurodegenerative disease is selected from the group consisting of Alzheimer's disease, Huntington's chorea, Parkinson's disease, multiple sclerosis, neuromyelitis optica, leukodystrophy, amyotrophic lateral sclerosis (ALS), degenerative ataxia, multiple system atrophy, neurodegeneration and brain iron accumulation disorders (NBIA), neuromyopathy, and a motor neuron disease, wherein the cerebrovascular disease is selected from the group consisting of global or local ischemia, intracerebral haemorrhage, stroke, and vascular dementia, or wherein
the viral disease is selected from the group consisting of meningitis, encephalitis, rabies, measles, mumps, poliomyelitis, herpes simplex, and varicella zoster.

59. The crystalline leriglitazone hydrate Form C of claim 58, wherein the leukodystrophy is X-linked adrenoleukodystrophy, adrenomyeloneuropathy, cerebral adrenoleukodystrophy, or metachromatic leukodystrophy, wherein
the degenerative ataxia is Friedreich's ataxia, wherein
the motor neuron disease is selected from the group consisting of progressive bulbar palsy, pseudobulbar palsy, primary lateral sclerosis (PLS), progressive muscular atrophy, spinal muscular atrophy (SMA), post-polio syndrome (PPS)-Marie-Tooth disease, Guillan-Barré syndrome, and adrenomyeloneuropathy (AMN).

60-83. (canceled)

84. A method of making crystalline leriglitazone hydrate Form C of claim 1, the method comprising the following steps:
(a) providing leriglitazone hydrochloride salt, preferably Form A, in water or in mixtures of water with an alcohol solvent to provide a solution or a suspension,
(b) optionally, heating the mixture of step (a) at a suitable temperature, preferably from 40° C. to 100° C., more preferably from 50° C. to 85° C.,
(c) optionally, cooling the solution or the suspension resulting from step (b) to room temperature, and
(d) isolating the solid obtained in step (c) providing Form C of leriglitazone.

85. A method of making crystalline leriglitazone Form B of claim 18, the method comprising the following steps:
(a) providing leriglitazone hydrochloride salt, preferably Form A, in water or in mixtures of water with an alcohol solvent to provide a solution or a suspension,
(b) optionally, heating the mixture of step (a) at a suitable temperature, preferably from 40° C. to 100° C., more preferably from 50° C. to 85° C.,
(c) optionally, cooling the solution or the suspension resulting from step (b) to room temperature, and
(d) isolating and drying the solid obtained in step (c) providing Form B of leriglitazone.

86. A kit comprising the pharmaceutical formulation of claim 34 in a container and (ii) a label with instructions how to use the kit.

87. The kit of claim 86, wherein the label is approved by the United States Food and Drug Administration (FDA), the European Medicines Agency (EMA), the China Food and Drug Administration (CFDA), or the Japanese Ministry of Health Labor and Welfare (MHLW).

88. (canceled)

89. A composition comprising 99.0 to 99.9 wt/wt % of leriglitazone (free base), leriglitazone hydrochloride, or leriglitazone hydrate, and:
(i) 0.0001 to 0.2 wt/wt % of (Z)-5-(4-(2-(5-(1-hydroxyethyl)pyridin-2-yl)ethoxy)benzylidene)thiazolidine-2,4-dione, or the hydrochloride salt thereof;
(ii) 0.0001 to 0.2 wt/wt % of 2,2′-disulfanediylbis(3-(4-(2-(5-(1-hydroxyethyl)pyridin-2-yl)ethoxy)phenyl) propanoic acid), or the hydrochloride salt thereof;
(iii) 0.0001 to 0.2 wt/wt % of 2-((1-hydroxy-3-(4-(2-(5-(1-hydroxyethyl)pyridin-2-yl)ethoxy)phenyl) propan-2-yl)disulfaneyl)-3-(4-(2-(5-(1-hydroxyethyl)pyridin-2-yl)ethoxy)phenyl) propanoic acid, or the hydrochloride salt thereof; and/or
(iv) 0.0001 to 0.15 wt/wt % of 5-(4-(2-(5-(1-hydroxyethyl)pyridin-2-yl)ethoxy)benzyl) thiazolidin-2-one, or the hydrochloride salt thereof.

90-92. (canceled)

93. A pharmaceutical formulation comprising:
(a) the composition of claim 89 in an amount from 0.01% w/v to 5% w/v;
(b) one or more cellulose derivatives in an amount from 0.01% w/v to 2% w/v, wherein the one or more cellulose derivatives have a viscosity between 1500 and 4500 mPa*s, preferably between 1500 to 3500 mPa*s, more preferably between 1500 and 3000 mPa*s as measured by Brookfield viscosity method; and
(c) water QS to 100%.
